(19) United States
(12) Patent Application Publication
(10) Pub. No.: US 2023/0149569 A1
(43) Pub. Date: May 18, 2023

(54) MULTIFUNCTIONAL COMPOUNDS FOR USE IN MEDICAL IMAGING AND THERAPY

(71) Applicant: Antelope Surgical Solutions, Inc., New York, NY (US)

(72) Inventors: Amy Wu, New York, NY (US); Richard Ting, New York, NY (US)

(21) Appl. No.: 17/935,084

(22) Filed: Sep. 23, 2022

Related U.S. Application Data
(60) Provisional application No. 63/249,180, filed on Sep. 28, 2021.

(51) Int. Cl. A61K 51/04; A61K 49/00; A61K 49/10

(52) U.S. Cl. CPC A61K 51/0482; A61K 49/0052; A61K 49/108

(57) ABSTRACT

The disclosure provides multifunctional compounds for use in medical imaging and therapy, the compounds comprising two or more of (i) a chelating ligand moiety (CL); (ii) an optical probe moiety (OP); and (iii) a biological targeting moiety (BT). The disclosure further provides related compositions and methods.

### CROSS-REFERENCES TO RELATED APPLICATIONS

This application claims the benefit of U.S. Provisional Application No. 63/249,180 filed Sep. 28, 2021, which is incorporated herein by reference in its entirety and for all purposes.

### FIELD OF THE INVENTION

The present invention relates to multifunctional compounds for use in medical imaging and therapy, including radiotherapy and radioimmunotherapy.

### BACKGROUND

The current standard of care in medical imaging utilizes a single agent for each of PET imaging, gadolinium contrast imaging (cMRI), optical/fluorescence imaging, and a further separate molecule for radio isotope therapy (RIT, or “radiotherapy”). In some cases, mixtures of several different molecules may be used, for example combined PET/optical, optical/RIT, or PET/RIT agents.

Single agent imaging agents such as PET-only, optical-only, or RIT-only imaging agents do not allow for corroborative imaging. Instead, each of the different molecules distributes differently in a patient, which often results in different signals originating from different, conflicting anatomical locations in a patient. For example, where multiple imaging agents are administered to a patient along with an RIT agent, such as a mixture of stand-alone PET/FL/RIT agents, a lesion may present as fluorescent and/or alpha/beta particle-emitting, but fail to present on the PET scan because the different agents distribute differently due, for example, to differences in blood clearance, non-specific tissue accumulation, ligand affinity, and receptor saturation. This leaves the surgeon/radiation oncologist/radiologist to reconcile the signal difference. Such conflicting information from multiple single imaging agents is very problematic in the operating room where time to resolve the conflict is limited and failure to obtain coherent imaging information may compromise therapy. Therapy would be compromised, for example, where a tumor margin is fluorescent but fails to present on the PET scan, or vice versa, where a spot on a PET scan is not fluorescent in the operating room.

Mixtures of imaging agents targeting the same ligand also suffer from the problem of signal dilution. The problem can be illustrated in two scenarios: 1) assume that a patient with a net 1 million PSMA ligand binding sites is being treated with a mixture of a PSMA-specific PET agent and a PSMA-specific RIT agent. If 50% of the ligand binding sites are bound to the PET agent and 50% of the sites are bound to the RIT agent, the PSMA positive tumor receives a theoretical maximum of only 50% of the maximum contrast by PET or 50% of the maximum RIT dose due to blocking of ligand binding sites with the PET molecule. 2) In a second scenario, a patient is first imaged with a PSMA-specific PET agent and then treated later with a subsequent PSMA-specific RIT agent. If the ligand binding sites become saturated (100% of the sites are bound) in imaging via the PET agent, a subsequent injected RIT dose will be ineffective or less effective due to PET agent blocking of available binding sites prior to RIT dose introduction.

### SUMMARY OF THE DISCLOSURE

The present invention addresses the problems associated with single-agent imaging agents and mixtures of single agent imaging agents with each other or with RIT agents by providing a single molecular agent comprising multiple functional modalities such as a fluorophore, a PET agent, an RIT agent, and optionally a biological targeting agent, such that a single molecule as described herein is suitable for performing optical fluorescence, PET imaging, and RIT imaging. The multifunctional compounds of the invention necessarily provide a superior signal to noise (S/N) ratio and/or radioisotope loading compared to mixtures of single agents with the same set of functionalities. In addition, the multifunctional compounds described here present advantages with respect to increased efficiencies for synthesis and regulatory approval, since the synthesis and testing of a single molecule (e.g., a single molecular species with the same sum atomic number, elemental connectivity, ¹H-NMR characterization) will simplify the FDA new drug application (NDA) process compared to filing for multiple separate molecules. In addition, the multifunctional compounds described here present advantages with respect to increased efficiencies for synthesis and regulatory approval compared to mixtures of single agents.

In addition, the multifunctional compounds described here provide molecularly-coherent PET and fluorescence images advantageous for image-guided surgery, including robotic and robot-assisted surgery. In embodiments, the disclosure further provides a surgical system for image-guided surgery comprising an in-surgical suite PET scanner (e.g., PET/CT or PET/MRI) equipped with a fluorescent endoscope or camera, located for example on a surgical robot or on a back table histopathology cart. The surgical system provides corroborative PET and fluorescent imaging during surgery, thereby improving patient therapy.

The present disclosure provides multifunctional compounds useful for medical imaging and therapy, including prior-, in-situ, and post-surgical medical imaging and therapy.

Provided are compounds or pharmaceutically acceptable salts thereof comprising three functional moieties (i) a chelating ligand moiety (CL); (ii) an optical probe moiety (OP); and (iii) a biological targeting moiety (BT); related compositions; and methods of use.

In an aspect, the compound comprises:
(i) a chelating ligand moiety (CL);
(ii) an optical probe moiety (OP); and
(iii) a biological targeting moiety (BT),
   wherein each of the moieties (i) to (iii) are bound by one or more linkers (e.g., L¹, L², L³, L⁴, L⁵, L⁶ and L⁷) disclosed herein to at least one other moiety to form a compound of Formula X. Also provided are, inter alia, compounds comprising four functional moieties (i) a fluorine atom-carrying moiety (FCM); (ii) a chelating ligand moiety (CL); (iii) an optical probe moiety (OP); and (iv) a biological targeting moiety (BT), related compositions, and methods of use.

In an aspect, the compound comprises:
(i) a fluorine atom-carrying moiety (FCM);
(ii) a chelating ligand moiety (CL);
(iii) an optical probe moiety (OP); and
(iv) a biological targeting moiety (BT),
wherein each of the moieties (i) to (iv) are bound by one or more linkers (e.g., L¹, L², L³, L⁴, L⁵, L⁶ and L⁷) disclosed herein to at least one other moiety to form a compound of Formula I.

In embodiments, the compound has a structure of Formula (I):

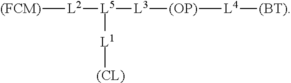

In some embodiments, the compound has a structure of Formula (I-a):

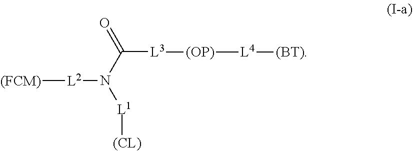

In some embodiments, the compound has a structure of Formula (II):

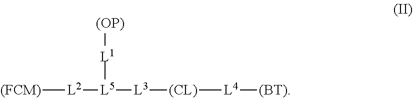

In some embodiments, the compound has a structure of Formula (III):

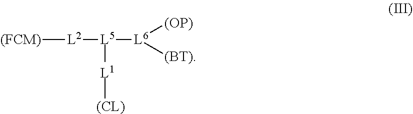

In some embodiments, the compound has a structure of Formula (IV-a) or (IV-b):

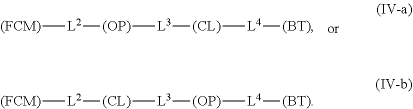

In some embodiments, the compound has a structure of Formula (V-a) or (V-b):

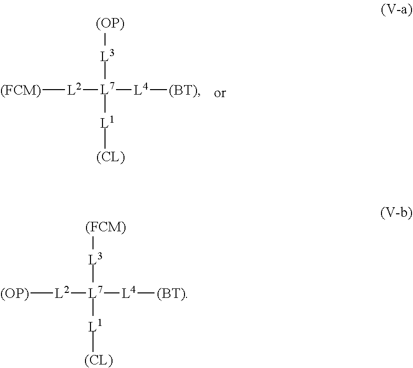

In some embodiments, the compound has a structure of Formula (VI-a) or (VI-b):

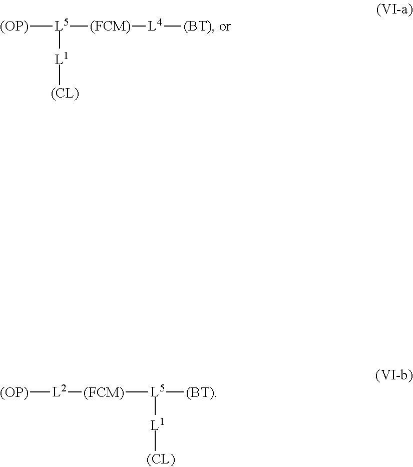

In an aspect, provided is a composition or pharmaceutical composition comprising a compound of Formula (I), (I-a), (I-a-1), (II), (III), (IV-a), (IV-b), (V-a), (V-b), (VI-a), (VI-b), (X), (X-a), (XI), (XII), (XIII-a), (XIII-b), (XIV-a), or (XIV-b), or pharmaceutically acceptable salt thereof, or a subembodiment described herein.

In an aspect, provided is a kit comprising a compound of Formula (I), (I-a), (I-a-1), (II), (III), (IV-a), (IV-b), (V-a), (V-b), (VI-a), (VI-b), (X), (X-a), (XI), (XII), (XIII-a), (XIII-b), (XIV-a), or (XIV-b), or pharmaceutically acceptable salt thereof, or a subembodiment described herein, in the form of a solid powder and a solid phase extraction device suitable for adsorbing labeled analyte; optionally further comprising one or more sterile solutions selected from purification, elution, washing, and neutralization solutions.

In an aspect, provided is a method for medical imaging in a subject. The method comprises: (i) administering to the subject a compound of Formula (I), (I-a), (I-a-1), (II), (III), (IV-a), (IV-b), (V-a), (V-b), (VI-a), (VI-b), (X), (X-a), (XI), (XII), (XIII-a), (XIII-b), (XIV-a), or (XIV-b), or pharmaceutically acceptable salt thereof, or a subembodiment described herein, or a pharmaceutical composition comprising same; ii) performing medical imaging of an internal biological tissue of the subject using a technique selected from at least one or two of positron emission tomography (PET), single photon emission computer tomography (SPECT), magnetic resonance imaging (MRI), contrast aided (e.g., gadolinium contrast) magnetic resonance imaging (cMRI), and fluorescence (FL) or absorbance-based optical imaging.

In an aspect, provided is a method for treating cancer in a subject using radioisotope therapy. The method comprises administering to the subject a compound of Formula (I), (I-a), (I-a-1), (II), (III), (IV-a), (IV-b), (V-a), (V-b), (VI-a), (VI-b), (X), (X-a), (XI), (XII), (XIII-a), (XIII-b), (XIV-a), or (XIV-b), or pharmaceutically acceptable salt thereof, or a subembodiment described herein, or a pharmaceutical composition comprising same; wherein the compound comprises a radioisotope suitable for radioisotope therapy.

In an aspect, the CL moiety of the compound comprises a radiometal suitable for radioisotope therapy and the method further comprises treating the subject with radioisotope therapy, such that imaging and radiotherapy are performed simultaneously with administration of a single compound of Formula (I), (I-a), (I-a-1), (II), (III), (IV-a), (IV-b), (V-a), (V-b), (VI-a), (VI-b), (X), (X-a), (XI), (XII), (XIII-a), (XIII-b), (XIV-a), or (XIV-b), or pharmaceutically acceptable salt thereof, or a subembodiment described herein.

In an aspect, the CL moiety of the compound comprises a metal suitable for contrast aided (e.g., gadolinium contrast) magnetic resonance (cMRI) or contrast enhanced computed tomography imaging.

In an aspect, the biological targeting moiety is selected from a blood cell (e.g., a red blood cell (RBD), a white blood cell (WBC), or a platelet), a peptide, a small molecule, a prodrug, a nucleic acid (e.g., DNA, or RNA), an aptamer, an oligosaccharide, and an antibody or antigen binding fragment thereof.

In an aspect, the BT moiety of the compound further comprises a drug or prodrug and the method further optionally comprises therapy with the drug or prodrug.

### BRIEF DESCRIPTION

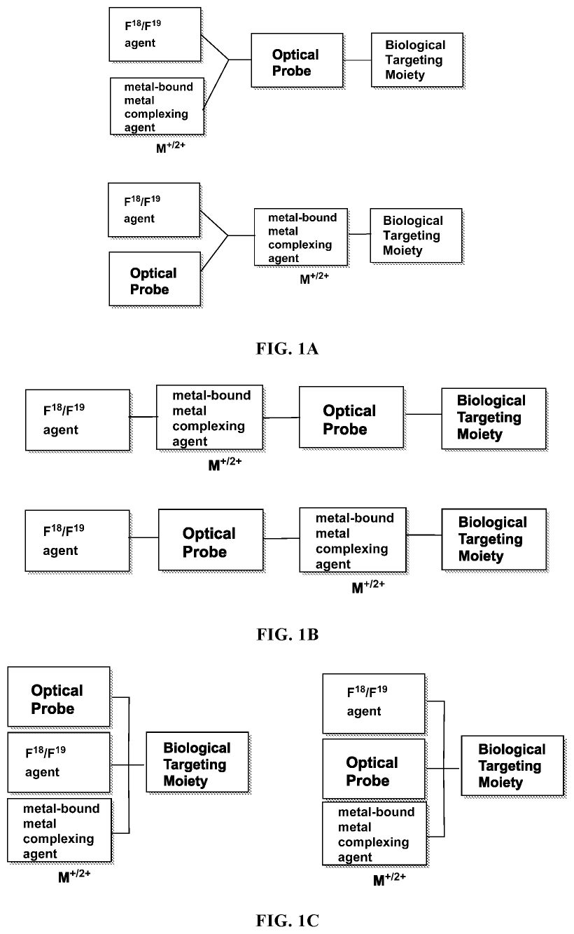

FIG. 1A-1C: exemplary arrangements of moieties in imaging agents including (i) a fluorine atom-carrying moiety (FCM); (ii) a chelating ligand moiety (CL); (iii) an optical probe moiety (OP); and (iv) a biological targeting moiety (BT).

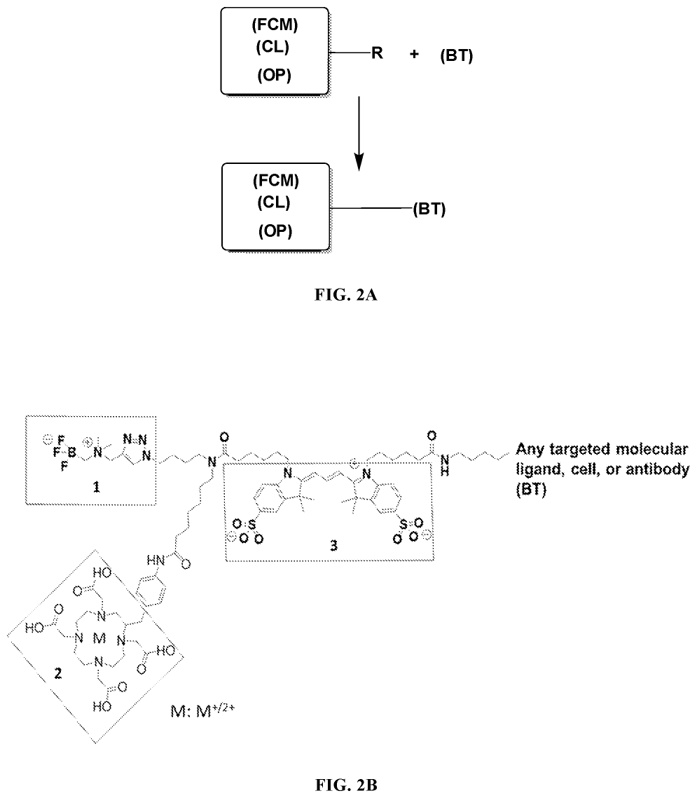

FIG. 2A-2B: A, scheme of attaching a biological targeting moiety (BT) via a reactive crosslinking group; B, an exemplary compound of Formula I. Box 1 shows the FCM, box 2 shows the CL, and box 3 shows the OP.

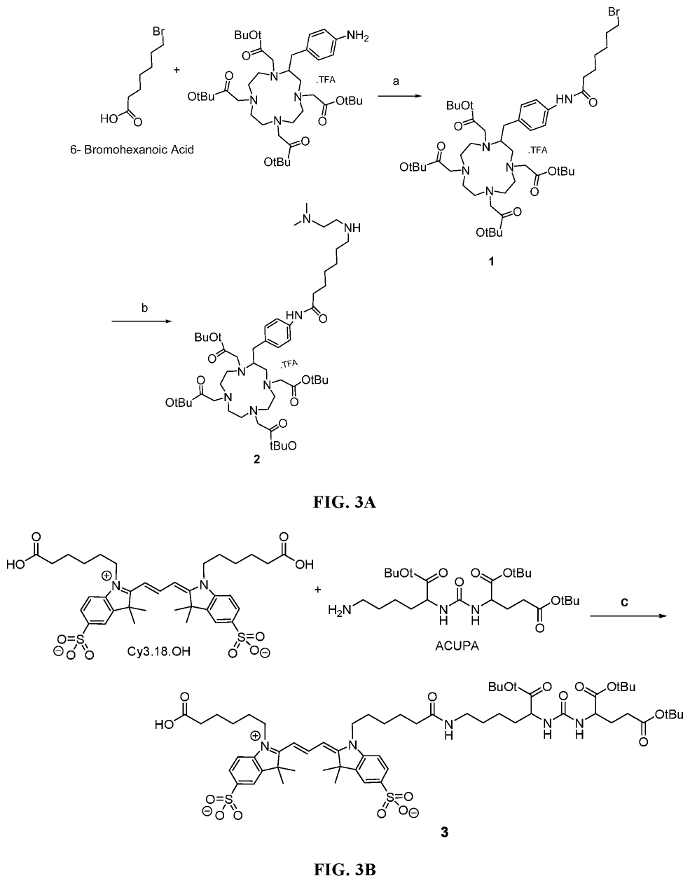

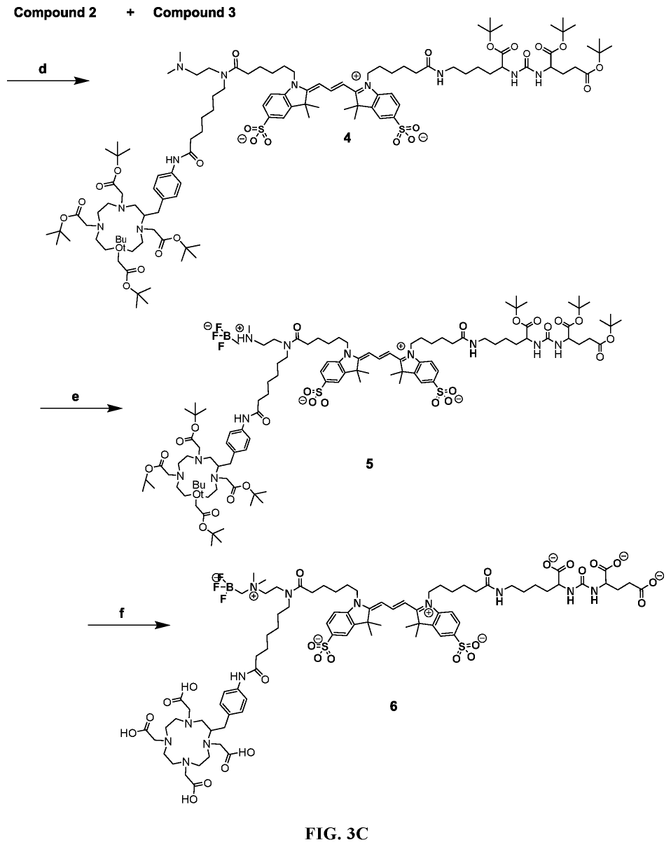

FIG. 3A-3C show exemplary synthesis reactions described in Example 1.

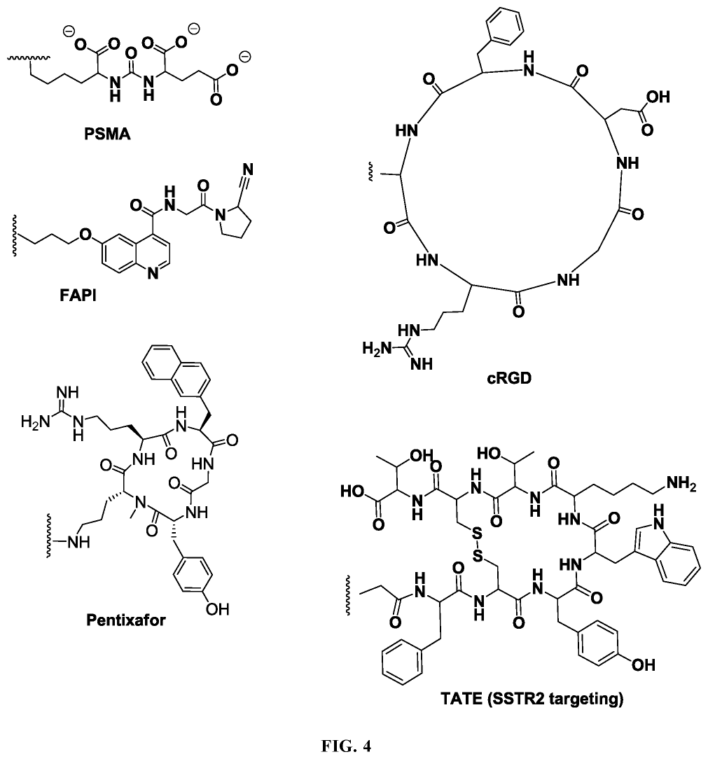

FIG. 4 shows exemplary biological targeting moieties (BT).

### DETAILED DESCRIPTION

Provided are compounds of Formula (I), (I-a), (I-a-1), (II), (III), (IV-a), (IV-b), (V-a), (V-b), (VI-a), (VI-b), (X), (X-a), (XI), (XII), (XIII-a), (XIII-b), (XIV-a), or (XIV-b), or pharmaceutically acceptable salt thereof, or a subembodiment described herein, compositions comprising same, and methods of use in medical imaging and therapy including PET imaging, optical/fluorescence imaging and metal-complex applications including gadolinium contrast imaging (cMRI), as well as radiation isotope therapy (RIT) and radioimmunotherapy.

In an aspect, the compound includes at least i) a chelating ligand moiety (CL); ii) an optical probe moiety (OP); iii) a biological targeting moiety (BT); and iv) a fluorine atom-carrying moiety (FCM) wherein the FCM comprises one or more fluorine atoms and one or more fluorine isotopes selected from Fluorine-18 (¹⁸F), preferably where the compound will be utilized for PET imaging, and Fluorine-19 (¹⁹F) for non-PET imaging.

In embodiments, the chelating ligand moiety (CL) may be bound to a metal ion or a metal that can be used in PET imaging, radioisotope therapy, or MRI contrast imaging. Examples of such metals include lutetium (Lu, e.g., ¹⁷⁵Lu or ¹⁷⁷Lu), actinium (Ac, e.g., ²¹⁷Ac, ²²⁵Ac), gallium (Ga, e.g., ⁶⁷Ga, or ⁶⁸Ga), copper (Cu), samarium (Sm), radium (Ra), yttrium (Y), palladium (Pd), iridium (Ir), gadolinium (Gd) or lead (Pb). In embodiments, ¹²⁵I and ⁶⁴Cu may be complexed with the compound for PET imaging. In embodiments, ⁹⁰Y may be complexed with the compound for SPECT imaging. In embodiments, gadolinium (Gd) is a stable isotope (i.e. non-radionuclide) for imaging (MRI contrast).

In embodiments, substitutions of atomic isotopes may be made, e.g. ¹⁸F for ¹⁹F, and ¹⁷⁵Lu for ¹⁷⁷Lu. These substitutions will not change the sum total of all the atomic numbers of atoms within the molecule/compound; however, these substitutions will change the atomic weight.

In embodiments, the fluorine atom-containing moieties (FCM) include, without limitation, fluorine captors. In embodiments, the FCM moiety permits visualization of the tissue of interest by PET imaging. In embodiments, the moiety contains either two or more fluorine atoms, which can be either ¹⁸F or ¹⁹F (“^{18/19}F”). In certain aspects, the FCM includes a fluorine atom-carrying moiety that may optionally function as a PET contrasting agent, by including ¹⁹F. In certain aspects, the FCM includes a fluorine atom-carrying moiety may include ¹⁹F, which is not a PET contrasting agent.

For example, a ¹⁸F-fluorophore-R molecule can be prepared by, for example, functionalizing a fluorophore with R reactive groups and a labile fluorine-containing group (e.g., —CR₃F, —Br:″ or —SiF₃) to produce a ¹⁹F-fluorophore-R molecule, and then contacting the ¹⁹F-fluorophore-R molecule with aqueous H[¹⁸F] under conditions (e.g., acidic pH, such as 2.5) where the ¹⁸F isotopically exchanges with ¹⁹F atoms, thereby resulting in at least one ¹⁸F per boron or silicon atom. The isotopic exchange method is described in, for example, U.S. Pat. No. 8,114,381, the contents of which are herein incorporated by reference.

In embodiments, the optical probe moiety (OP) may comprise any type of molecule suitable for fluorescence or optical contrast (absorbance) imaging. In embodiments, the optical probe comprises a fluorophore or other highly photon-absorbing agent such as indocyanine green, tri-, penta- or heptamethine cyanine, fluorescein, rhodamine, or Evan's blue dye.

As used herein, the term “fluorophore” (or “fluorescing species”) refers to a compound possessing a fluorescent property when appropriately stimulated by electromagnetic radiation. The fluorophores considered herein can absorb and emit light of any suitable wavelength. In embodiments, it may be desired to select a fluorophore with particular absorption and emission characteristics. For example, in different embodiments, the fluorophore absorbs at nanometer (nm) wavelengths of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, or 800 nm, or within a range bounded by any two of the foregoing values. In certain embodiments, the fluorophore emits at any of the foregoing wavelengths, or within a range bounded by any two of the foregoing values, wherein it is understood that a fluorophore generally emits at a longer wavelength than the absorbed wavelength. The impinging electromagnetic radiation (i.e., which is absorbed by the fluorophore) can be in a dispersed form, or alternatively, in a focused form, such as a laser. Moreover, the absorbed or emitted radiation can be in the form of, for example, far infrared, infrared, far red, visible, near-ultraviolet, or ultraviolet.

The fluorophores considered herein are organic fluorophores, which generally contain at least one carbon-carbon bond and at least one carbon-hydrogen bond. In different embodiments, the organic fluorophore can include, for example, a charged (i.e., ionic) molecule (e.g., sulfonate or ammonium groups), uncharged (i.e., neutral) molecule, saturated molecule, unsaturated molecule, cyclic molecule, bicyclic molecule, tricyclic molecule, polycyclic molecule, acyclic molecule, aromatic molecule, and/or heterocyclic molecule (i.e., by being ring-substituted by one or more heteroatoms selected from, for example, nitrogen, oxygen and sulfur). In the particular case of unsaturated fluorophores, the fluorophore contains one, two, three, or more carbon-carbon and/or carbon-nitrogen double and/or triple bonds. In a particular embodiment, the fluorophore contains at least two (e.g., two, three, four, five, or more) conjugated double bonds (i.e., a polyene linker) aside from any aromatic group that may be in the fluorophore. In embodiments, the fluorophore is a fused polycyclic aromatic hydrocarbon (PAH) containing at least two, three, four, five, or six rings (e.g., naphthalene, pyrene, anthracene, chrysene, triphenylene, tetracene, azulene, and phenanthrene) wherein the PAH can be optionally ring-substituted or derivatized by one, two, three or more heteroatoms or heteroatom-containing groups. In embodiments, the fluorophore contains a polyalkyleneoxide group that contains at least two, three, or four alkyleneoxide units. In embodiments, the fluorophore contains at least one sulfonic acid or sulfonate salt group.

In embodiments, the organic fluorophore is a xanthene derivative (e.g., fluorescein, rhodamine, Oregon green, eosin, and Texas Red), cyanine or its derivatives or subclasses (e.g., streptocyanines, hemicyanines, closed chain cyanines, phycocyanins, allophycocyanins, indocarbocyanines, oxacarbocyanines, thiacarbocyanines, merocyanins, and phthalocyanines), naphthalene derivatives (e.g., dansyl and prodan derivatives), coumarin and its derivatives, oxadiazole and its derivatives (e.g., pyridyloxazoles, nitrobenzoxadiazoles, and benzoxadiazoles), pyrene and its derivatives, oxazine and its derivatives (e.g., Nile Red, Nile Blue, and cresyl violet), acridine derivatives (e.g., proflavin, acridine orange, and acridine yellow), arylmethine derivatives (e.g., auramine, crystal violet, and malachite green), and tetrapyrrole derivatives (e.g., porphyrins and bilirubins).

In embodiments, the fluorophore includes a moiety of the following formula,

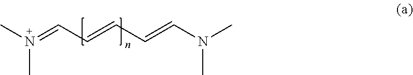

wherein n is an integer of 0 to 12. Other structures related to or derived from formula (1) are also considered herein, as amply described in Guieu, V., et al., Eur. J. Org. Chem., 2007, 804-810, which is incorporated herein by reference in its entirety.

In embodiments, the fluorophore includes a moiety of the following formula,

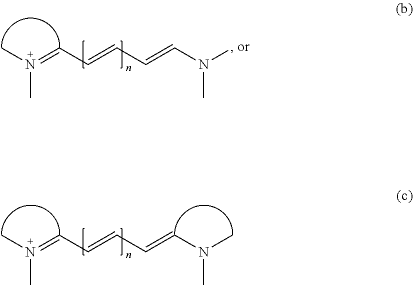

wherein n in formula (2) is as defined above. The arc in Formula (2) indicates a nitrogen-containing ring, such as pyrrolyl. The arc may alternatively represent a bicyclic ring system, such as a benzopyrrolyl fused ring system. Other structures related to or derived from formula (2) are also considered herein, as amply described in Stathatos, E., et al. Chem. Mater., 2001, 13, 3888-3892, and Yao, Q.-H., et al. J. Mater. Chem., 2003, 13, 10481053, which are incorporated herein by reference in their entirety.

In embodiments, the fluorophore includes a cyanine dye (i.e., cyanine-based fluorophore). The term “cyanine dye”, as used herein, refers to any of the dyes, known in the art, that include two indolyl or benzoxazole ring systems interconnected by a conjugated polyene linker. The cyanine dye typically contains at least two or three conjugated carbon-carbon double bonds, at least one of which is not in a ring, such as depicted in any of Formulas (1)-(3). The cyanine dye (or other type of dye) often contains at least two pyrrolyl rings. Some particular examples of cyanine dyes are the Cy* family of dyes, which include, for example, Cy2, Cy3, Cy3B, Cy3.5, Cy5, Cy5.5, Cy7, and Cy9. The term “cyanine moiety”, as used herein, generally includes the bis-indolyl-polyene or bis-benzoxazolyl-polyene system, but excludes groups attached to the ring nitrogen atoms in the indolyl or benzoxazolyl groups. The cyanine dyes may also include the Alexa® family of dyes (e.g., Alexa Fluor 350, 405, 430, 488, 500, 514, 532, 546, 555, 568, 594, 610, 633, 647, 660, 700, 750, and 790, the ATTO® family of dyes (e.g., ATTO 390, 425, 465, 488, 495, 520, 532, 550, 565, 590, 594, 601, 615, 619, 629, 635, 645, 663, 680, 700, 729, and 740), and the Dye′ family of dyes (e.g., DY 530, 547, 548, 549, 550, 554, 556, 560, 590, 610,615, 630, 631, 631, 632, 633, 634, 635, 636, 647, 648, 649, 650, 651, 652, 675, 676, 677, 680, 700, 701, 730, 731, 732, 734, 750, 751,752, 776, 780, 781, 782, and 831). The ATTO dyes, in particular, can have several structural motifs, including, coumarin-based, rhodamine-based, carbopyronin-based, and oxazine-based structural motifs.

In embodiments, the fluorophore permits visualization of the tissue of interest by, for example, fluorescence imaging and “optical” imaging (such as visual observation with the naked eye). Fluorophores include, for example, Cy3, Cy7, fluorescein, and any of the fluorophores known in the art, such as those described above. In embodiments, the fluorophore is preferably a cyanine fluorophore, and more particularly, a hydrophilic cyanine fluorophore.

In embodiments, the biological targeting moiety (BT) is selected from a blood cell (e.g., a red blood cell (RBD), a white blood cell (WBC), or a platelet), a peptide, a small molecule, a prodrug, a nucleic acid (e.g., a DNA, or RNA molecule), an aptamer, an oligosaccharide, and an antibody or antigen binding fragment thereof.

In embodiments, the BT is an agent that specifically binds to a biological molecule such as a cell-surface receptor or ligand. In embodiments, the BT is selected from a PSMA inhibitor such as 2-(3-((S)-5-amino-1-carboxypentyl)ureido)pentanedioic acid; a fibroblast activation protein (FAP) inhibitor such as 6-butoxy-N-(2-(2-cyanopyrrolidin-1-yl)-2-oxoethyl)quinoline-4-carboxamide or 6-butoxy-N-(2-(2-cyano-4,4-difluoropyrrolidin-1-yl)-2-oxoethyl)quinoline-4-carboxamide; an arginine-glycine-glutamate fibronectin or integrin binding peptide such as 2-(5-benzyl-11-(3-guanidinopropyl)-8-methyl-3,6,9,12,15-pentaoxo-1,4,7,10,13-pentaazacyclopentadecan-2-yl)acetic acid (RGD); a somatostatin binding peptide such as 2-(13-((1H-indol-3-yl)methyl)-10-(4-aminobutyl)-16-(4-hydroxybenzyl)-7-(1-hydroxyethyl)-6,9,12,15,18-pentaoxo-19-(3-phenyl-2-propionamidopropanamido)-1,2-dithia-5,8,11,14,17-pentaazacycloicosane-4-carboxamido)-3-hydroxybutanoic acid (TATE) or 13-((1H-indol-3-yl)methyl)-10-(4-aminobutyl)-N-(1,3-dihydroxybutan-2-yl)-16-(4-hydroxybenzyl)-7-(1-hydroxyethyl)-6,9,12,15,18-pentaoxo-19-(3-phenyl propionamidopropanamido)-1,2-dithia-5,8,11,14,17-pentaazacycloicosane-4-carboxamide (TOC); a pentixafor chemokine receptor binding agent such as 1-(3-((2S,5S,11R,14R)-11-(4-hydroxybenzyl)-13-methyl-14-(3-(methylamino)propyl)-5-(naphthalen-2-ylmethyl)-3,6,9,12,15-pentaoxo-1,4,7,10,13-pentaazacyclopentadecan-2-yl)propyl)guanidine or related variants.

In embodiments, the BT is an antibody or antigen binding fragment thereof. In embodiments, the antibody may consist of an immunoglobulin (Ig) molecule, such as an IgG molecule. In embodiments, the antibody may be an antigen binding fragment of an Ig molecule, such as a F(ab′)2 or Fab′ fragment, or a single chain FAT fragment (scFv). In embodiments, the antibody or antigen binding fragment thereof may be a humanized antibody. In embodiments, the antibody or antigen binding fragment thereof is a humanized single chain heavy-chain antibody (HcAb) consisting of two heavy chains attached to antigen-binding variable domains (variable heavy homodimers, VHH). In embodiments, the HcAb may be derived from a camelid, such as a camel or llama, or a cartilaginous fish (Chondrichthyes), such as a shark.

In embodiments, the BT is an antibody or antigen binding fragment thereof that binds directly to a molecule displayed on the cell surface of a target cancer cell, or intracellularly within the target cancer cell. In embodiments, the BT is an antibody selected from Herceptin, annexin, and Erbitux, or an antigen binding fragment thereof.

In embodiments, the molecule targeted for binding by the BT is selected from PD-L1, HER2/neu (the receptor tyrosine kinase also referred to as erbB-2, CD340, proto-oncogene Neu, Erbb2, or ERBB2, associated primarily with breast cancer), epidermal growth factor receptor (EGFR, found in multiple cancers), fibroblast activation protein (FAP, found in multiple cancers), a C-X-C chemokine receptor (CXCR, found in multiple cancers), somatostatin receptor 2 (SSTR2, found primarily in endocrine tissue derived cancers, including neuroendocrine cancers such as small-cell lung cancer, or SCLC), and epithelial cellular adhesion molecule (EPCAM, found multiple cancers).

In embodiments, the biological targeting moiety (BT) is a radiolabelled antibody or antigen binding fragment thereof suitable for radioimmunotherapy. Suitable radionuclides for radioimmunotherapy include beta emitters, such as ⁹⁰Yttrium, ¹³¹Iodine, ¹⁷⁷Lutetium, ¹⁸⁸Rhenium, and ⁶⁷Copper; alpha emitters, such as ²¹³Bisumth, ²¹¹Astatine, and ²²⁵Actinium; and Auger-electron emitters such as ¹²⁵Iodine.

In embodiments, the biological targeting moiety (BT) is a protein or peptide such as annexin engineered to bind to a specific biological molecule, e.g., the programmed cell death-ligand 1 (PD-L1).

In embodiments, the biological targeting moiety (BT) is selected from somatostatin, the SSR agonist tyrosine-octreotate (TATE), and the SSTR2 targeting moiety, TOC, each of which is suitable for binding to SSTR2.

In embodiments, the biological targeting moiety (BT) is pentixafor, which is suitable for chemokine targeting, e.g., CXCR4.

In embodiments, the biological targeting moiety (BT) is a nucleic acid-based molecule, such as Pegaptanib sodium/Macugen (VEGF targeting), E10030 (PDGF), ARC1905 (C5), AS1411 (Nucleolin), NOX-A12 (CXCL12), NOX-E36 (CCL2), NOX-H94 (Hepcidin), ARC1779 (vWF), NU172 (FIXa), BX499 (TFPI).

In embodiments, the biological targeting moiety (BT) may be a prodrug or derivative thereof such as aciclovir, fluorouracil, cyclophosphamides, diethylstilbenstrols, DOPA, mercaptopurines, mitomycin, zidovudine, carbamazepine, captopril, carisoprodol, heroin, molsidomine, leflunomide, paliperidone, phenacetin, primidone, psilocybin, sulindac, fursultiamine, codeine Loperamide oxide, oxyphenisatin, sulfasalazine, Acetylsalicylate, bacampicillin, bambuterol, chloramphenicol succinate, dipivefrin, fosphenytoin, lisdexamfetamine, pralidoxime, ADEPTs, GDEPTs, VDEPTs. In embodiments, the BT moiety includes a prodrug that readily undergoes chemical changes under physiological conditions to provide a therapeutically effective chemical reagent (e.g., inhibitor, agonist, modulator, or regulator). In embodiments, the prodrugs of the compounds described herein may be converted in vivo after administration, or can be converted to the therapeutically effective chemical reagent by chemical or biochemical methods in an ex vivo environment, such as, for example, when contacted with a suitable enzyme or chemical reagent.

In embodiments, the biological targeting moiety (BT) is an oligosaccharide such as chitosan oligosaccharides (anti-inflammatory, anti-bacterial activity), fibrinogen oligosaccharides (blood coagulation, venous trhomboembolism).

In embodiments, the biological targeting moiety (BT) is a cell, particularly a blood cell such as a red blood cell or a platelet. Methods of conjugating a cell to a compound described herein are provided infra in the discussion of reactive crosslinking groups.

In embodiments, the biological targeting moiety (BT) is bound to the other moieties using a scheme as shown in FIG. 2A. The “R” group represents a reactive crosslinking group capable of binding to a blood cell. Exemplary reactive crosslinking groups include amino-reactive, carboxy-reactive, thiol-reactive, alcohol-reactive, phenol-reactive, aldehyde-reactive, and ketone-reactive groups. Amino-reactive groups may include carboxy groups (—COOR′, where R′ is H or hydrocarbon group), activated ester groups (—COOR′, where R′ is a carboxy-activating group, such as deprotonated N-hydroxysuccinimide, i.e., NHS), carbodiimide ester groups (e.g., EDC), tetrafluorophenyl esters, dichlorophenol esters, epoxy (e.g., glycidyl) groups, isothiocyanate, sulfonylchloride, dichlorotriazines, aryl halides, and azide, and sulfo-derivatives thereof, and combinations thereof. Carboxy-reactive groups may include amino groups and hydroxyalkyl groups, typically in the presence of a carboxy group activator to form an activated ester. Some examples of thiol-reactive groups include maleimido (“Mal”) groups, haloacetamide (e.g., iodoacetamide) groups, disulfide groups, thiosulfate, and acryloyl groups. Alcohol-reactive and phenol-reactive groups may include aldehydes, ketones, haloalkyl, isocyanate, and epoxy (e.g., glycidyl) groups. Aldehyde-reactive and ketone-reactive groups may include phenol, hydrazide, semicarbazide, carbohydrazide, and hydroxylamine groups. Other reactive groups include 6-oxyguanine groups and phosphoramidite groups. The term “reactive group” can further encompass any larger group (e.g., a hydrocarbon group, such as a cyclic or aromatic hydrocarbon) on which the reactive crosslinking group is attached. For example, a 6-oxyguanine group may include a ring-containing linking moiety attached to the 6-oxy atom for attaching to the linking portion. In other embodiments, the reactive group may be derivatized, such as by including any of the hydrophilic groups described above, such as sulfonate (e.g., a sulfo-NHS group), carboxy, hydroxy, or halide groups.

### Compounds

The abbreviations used herein have their conventional meaning within the chemical and biological arts (chemical biology). The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like.

As used herein, the term “alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups can be substituted or unsubstituted. In embodiments, alkylene groups are substituted with 1-2 substituents. As a non-limiting example, suitable substituents include halogen and hydroxyl.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)2R′— represents both —C(O)2R′— and —R′C(O)2-. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

As used herein, “cycloalkyl” refers to a saturated ring assembly containing from 3 to 10 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈. Cycloalkyl rings can be saturated or unsaturated, when unsaturated cycloalkyl rings can have one or two double bonds. Cycloalkyl rings include, for example, cyclopropyl, cyclobutyl, cyclopentyl, cyclohexyl, and cyclooctyl. Cycloalkyl groups can be substituted or unsubstituted. In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)w, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane.

In embodiments, a heterocycloalkyl is a heterocyclyl. As used herein, the term“heterocyclyl”, “heterocyclic”, or “heterocycloalkyl” refers to a heterocyclic group that is saturated or partially saturated and is a monocyclic or a polycyclic ring; which has 3 to 16, most preferably 5 to 10 and most preferably 1 or 4 ring atoms; wherein one or more, preferably one to four, especially one or two ring atoms are a heteroatom selected from oxygen, nitrogen and sulfur (the remaining ring atoms therefore being carbon). The term heterocyclyl excludes heteroaryl. The heterocyclic group can be attached to the rest of the molecule through a heteroatom, selected from oxygen, nitrogen and sulfur, or a carbon atom. The heterocyclyl can include fused or bridged rings as well as spirocyclic rings. Examples of heterocyclyl include dihydrofuranyl, dioxolanyl, dioxanyl, dithianyl, piperazinyl, pyrrolidine, dihydropyranyl, oxathiolanyl, dithiolane, oxathianyl, thiomorpholino, oxiranyl, aziridinyl, oxetanyl, oxepanyl, azetidinyl, tetrahydrofuranyl, tetrahydrothiophenyl, pyrrolidinyl, tetrahydropyranyl, piperidinyl, morpholine, piperazinyl, azepinyl, oxapinyl, oxaazepanyl, oxathianyl, thiepanyl, azepanyl, dioxepanyl, and diazepanyl.

As used herein, the term “halogen” or “halo” refers to fluorine, chlorine, bromine and iodine.

As used herein, the term “aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Additional heteroatoms can also be useful, including, but not limited to, B, Al, Si and P. Heteroaryl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 9 ring members and from 1 to 4 heteroatoms, or from 5 to 9 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), purine. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

The symbol “ [symbol] ” denotes the point of attachment of a chemical moiety to the remainder of a molecule or chemical formula.

The term “oxo” as used herein, means an oxygen atom connected to the point of attachment by a double bond (═O). The term “thio” as used herein, means a sulfur atom connected to the point of attachment by a double bond (═S).

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

A “substituent group,” as used herein, means a group selected from the following moieties:
(A) oxo, thio, halogen, —CCl₃, —CBr₃, —CF₃, —CI₃, —CH₂Cl, —CH₂Br, —CH₂F, —CHCl₂, —CHBr₂, —CHF₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, thio, halogen, —CCl₃, —CBr₃, —CF₃, —CI₃, —CH₂Cl, —CH₂Br, —CH₂F, —CH₂I, —CHCl₂, —CHBr₂, —CHF₂, —CHI2, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from the groups in (i).

Certain compounds of the present disclosure possess asymmetric carbon atoms (optical centers) or double bonds; the racemates, diastereomer, geometric isomers, regioisomers and individual isomers (e.g., separate enantiomers) are all intended to be encompassed within the scope of the present disclosure. In embodiments, the compounds of the present disclosure are a particular enantiomer, anomer, or diastereomer substantially free of other forms.

The terms “a”, “an” and “the” as used in herein means one or more, and are intended to include the plural forms as well, unless the context clearly indicates otherwise.

For example, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Descriptions of compounds of the present disclosure or pharmaceutically acceptable salts thereof are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “solution” refers to a liquid mixture in which the minor component (e.g., a solute or compound) is uniformly distributed within the major component (e.g., a solvent).

The terms “bind”, “bound”, “affix”, “affixed”, “attach”, or “attached” as used herein is used in accordance with its plain and ordinary meaning and refers to the association between atoms or molecules. The association can be direct or indirect. For example, bound atoms or molecules may be direct, e.g., by covalent bond or linker (e.g. a first linker or second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

The term “pharmaceutically acceptable salts” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

In this disclosure, “comprises,” “comprising,” “containing” and “having” and the like can have the meaning ascribed to them in U.S. Patent law and can mean “includes,” “including,” and the like. “Consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

All starting materials, building blocks, reagents, acids, bases, dehydrating agents, solvents, and catalysts utilized to synthesis the compounds of the present disclosure (e.g., Example 1) are either commercially available or can be produced by organic synthesis methods known to one of ordinary skill in the art.

In an aspect, provided is a compound, or pharmaceutically acceptable salt thereof, including the following moieties:
(i) a chelating ligand moiety (CL);
(ii) an optical probe moiety (OP); and
(iii) a biological targeting moiety (BT).

In embodiments, the compound further comprises a fluorine atom-carrying moiety (FCM).

In embodiments, the compound does not comprise a fluorine atom-carrying moiety (FCM).

In an aspect, provided is a compound including the following moieties:
(i) a fluorine atom-carrying moiety (FCM);
(ii) a chelating ligand moiety (CL);
(iii) an optical probe moiety (OP); and
(iv) a biological targeting moiety (BT);
or a pharmaceutically acceptable salt thereof.

In embodiments, the compound further includes a metal ion. In embodiments, the metal ion binds to the chelating ligand (CL). In embodiments, the metal ion is selected from a radioactive or non-radioactive isotope of a metal selected from Y, I, Lu, Sm, Re, Re, Cu, Pb, Ho, Sc, Ac, Bi, Bi, At, Pb, Th, and Ra. In embodiments, the metal ion is an cation of ¹⁷⁷Lu, ²²⁵Ac, or gadolinium (Ga). In embodiments, the metal ion is ¹²⁵I or ⁶⁴Cu. In embodiments, a compound comprising a CL moiety bound to ¹²⁵I or ⁶⁴Cu is particularly useful for PET imaging. In embodiments, the metal ion is ⁹⁰Y. In embodiments, a compound comprising a CL moiety bound to ⁹⁰Y is particularly useful for SPECT imaging.

In embodiments, each of the moieties of (i) to (iv) of a compound of Formula I are bound to at least one other moiety by one or more linkers selected from L¹, L², L³, L⁴, L⁵, L⁶ and L⁷.

L¹ is a bond, -L^{1A}, -L^{1A}L^{1B}-, -L^{1A}L^{1B}-L^{1C}-, -L^{1A}C(O)NR¹¹L^{1B}-, -L^{1A}NR¹¹L^{1B}-, -L^{1A}C(O)L^{1B}-, -L^{1A}C(O)OL^{1B}-, -L^{1A}OC(O)L^{1B}-, -L^{1A}(OL^{1B})ₙ₁-, -L^{1A}NR¹¹C(O)L^{1B}-, -L^{1A}NR¹¹C(O)OL^{1B}-, or -L^{1A}NR¹¹(OL^{1B})ₙ₁-;

L² is a bond, -L^{2A}, -L^{2A}L^{2B}, -L^{2A}L^{2B}-L^{2C}-, -L^{2A}C(O)NR¹²L^{2B}-, -L^{2A}NR¹²L^{2B}-, -L^{2A}C(O)L^{2B}-, -L^{2A}C(O)OL^{2B}-, -L^{2A}OC(O)L^{2B}-, -L^{2A}(OL^{2B})ₙ₂-, -L^{2A}NR¹²C(O)L^{2B}-, -L^{2A}NR¹²C(O)OL^{2B}-, or -L^{2A}NR¹²(OL^{2B})ₙ₂-;

L³ is a bond, -L^{3A}, -L^{3A}L^{3B}- -L^{3A}L^{3B}-L^{3C}-, -L^{3A}C(O)NR¹³L^{3B}, -L^{3A}NR¹³L^{3B}-, -L^{3A}C(O)L^{3B}-, -L^{3A}C(O)OL^{3B}-, -L^{3A}OC(O)L^{3B}-, -L^{3A}(OL^{3B})ₙ₃-, -L^{3A}NR¹³C(O)L^{3B}-, -L^{3A}NR¹³C(O)OL^{3B}-, or -L^{3A}NR¹³(OL^{3B})ₙ₃-;

L⁴ is a bond, -L^{4A}, -L^{4A}L^{4B}-, -L^{4A}L^{4B}-L^{4C}-, -L^{4A}C(O)NR¹⁴L^{4B}-, -L^{4A}NR¹⁴L^{4B}-, -L^{4A}C(O)L^{4B}-, -L^{4A}C(O)OL^{4B}-, -L^{4A}OC(O)L^{4B}-, -L^{4A}(OL^{4B})ₙ₄-, -L^{4A}NR¹⁴C(O)L^{4B}-, -L^{4A}NR¹⁴C(O)OL^{4B}-, or -L^{4A}NR¹⁴(OL^{4B})ₙ₄-.

L⁵ is

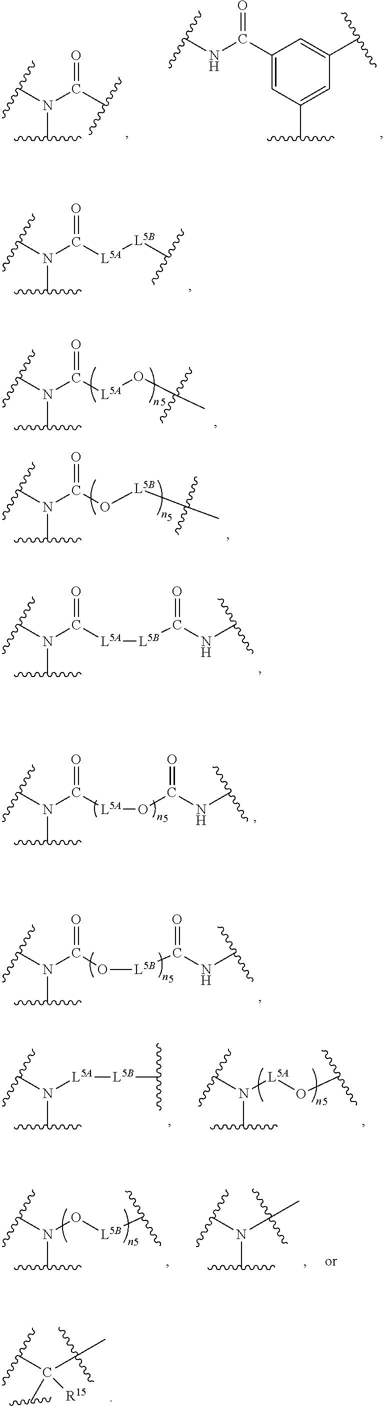

L⁶ is

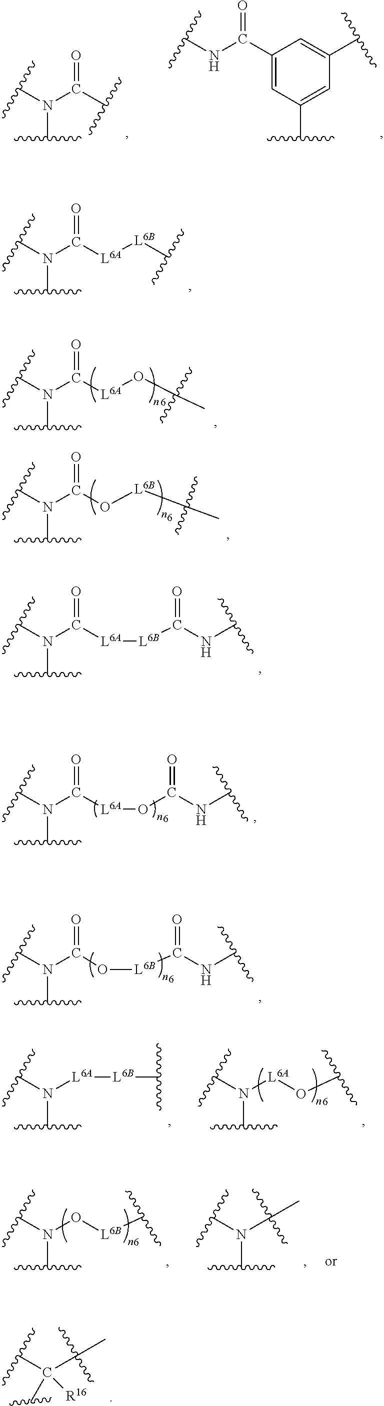

L⁷ is

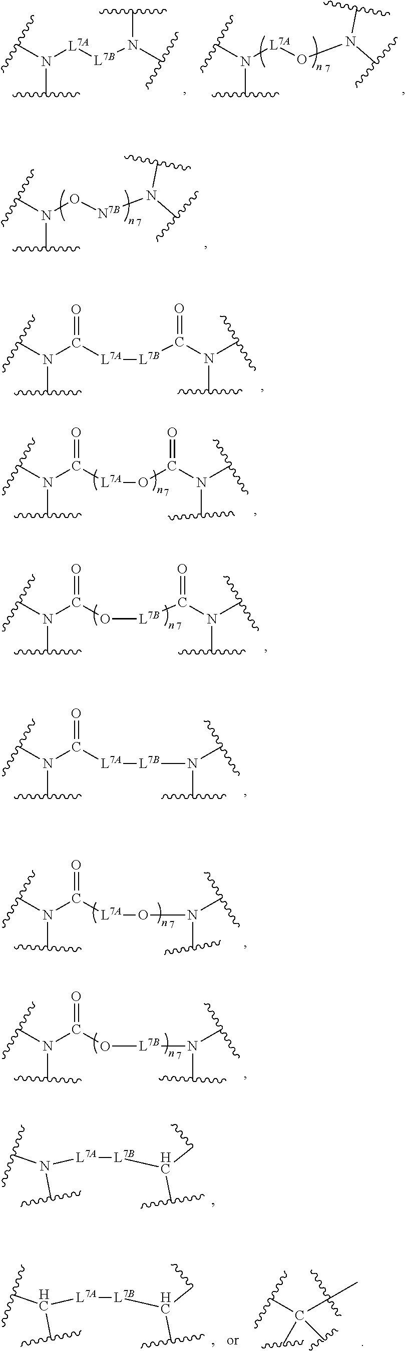

Each L^{1A}, L^{1B}, L^{1C}, L^{2A}, L^{2B}, L^{2C}, L^{3A}, L^{3B}, L^{3C}, L^{4A}, L^{4B}, L^{4C}, L^{5A}, L^{5B}, L^{6A}, L^{6B}, L^{7A}, and L^{7B} is independently a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Each R¹¹, R¹², R¹³, R¹⁴, R¹⁵, and R¹⁶ is independently hydrogen, and unsubstituted alkyl.

Each n1, n2, n3, n4, n5, n6 and n7 is independently an integer from 0 to 20.

In embodiments, each L^{1A}, L^{1B}, L^{1C}, L^{2A}, L^{2B}, L^{2C}, L^{3A}, L^{3B}, L^{3C}, L^{4A}, L^{4B}, and L^{4C} is independently a bond, unsubstituted C₁-C₁₂ alkylene, unsubstituted 2 to 12 membered heteroalkylene, unsubstituted C₃-C₁₂ cycloalkylene, unsubstituted 5 to 12 membered heterocycloalkylene, unsubstituted phenylene, or unsubstituted 5 to 12 membered heteroarylene.

In embodiments, the FCM includes one or more of F¹⁸ and F¹⁹. In embodiments, the FCM including —BF₂— and/or —BF₃ including one or more of F¹⁸ and F¹⁹. In embodiments, the FCM includes, for example,

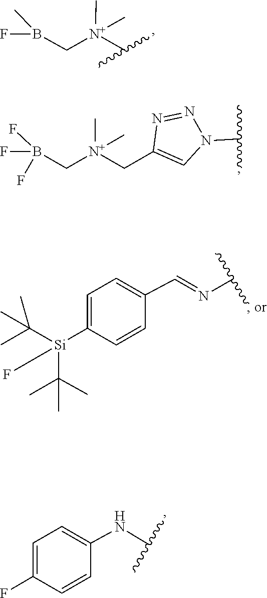

and one or more of F is F¹⁸/F¹⁹.

In embodiments, the FCM includes a —BF₂— and/or —BF₃ moiety, and the —BF₂— and/or —BF₃ moiety comprises two or more of F¹⁸. In embodiments, the —BF₂— moiety includes two F¹⁸. In embodiments, the —BF₃ moiety includes two F¹⁸. In embodiments, the —BF₃ moiety includes three F¹⁸.

In embodiments, the FCM includes a —BF₂— and/or —BF₃ moiety, and the —BF₂— and/or —BF₃ moiety comprises two or more of F¹⁹. In embodiments, the —BF₂— moiety includes two F¹⁹. In embodiments, the —BF₃ moiety includes two F¹⁹. In embodiments, the —BF₃ moiety includes three F¹⁹.

In embodiments, the OP includes one or more of fluorophores as described herein. In embodiments, the OP includes a cyanine-based fluorophore and a xanthene-based fluorophore. In embodiments, the OP includes one or more fluorescent or light-absorbing dyes and their derivatives. In embodiments, the OP includes Cy2, Cy3, Cy3B, Cy3.5, Cy5, Cy5.5, Cy7, or Cy9 (cyanine-based fluorophores). In embodiments, the OP includes rhodamine. In embodiments, the OP includes azo dyes such as Evans blue (tetrasodium salt of 6,6′-{(3,3′-dimethyl[1,1′-biphenyl]-4,4′-diyl)bis[diazene-2,1-diyl]}bis(4-amino hydroxynaphthalene-1,3-disulfonate) and isosulfan blue (lymphazurin).

In embodiments, the BT includes one or more selected from small molecule, prodrug, cell, blood cell, peptide, oligosaccharide, nucleic acid, aptamer, targeting agent, antibody, and antibody fragment. In embodiments, the BT include, but is not limited to, one or more selected from a prostate-specific membrane antigen (PSMA)-targeting agent, fibroblast activation protein (FAP) inhibitor, fibronectin or integrin targeted agent, somatostatin targeted peptide, Pentixafor chemokine receptor, antibody, antibody fragment, reengineered antibody T-cell, and heparin. Exemplary BT are shown in FIG. 4 but the examples are not limited thereto.

In embodiments, the BT does not include a divalent or trivalent counter cation. In embodiments, the BT may include single reactive amine. In embodiments, the BT in the synthesis of the compounds as described herein may contain one or more amines necessary for its biological function. In embodiments, the BT may include an acid group (e.g., carboxylic acid group) which is chemically protected. In embodiments, the BT may not contain non-protected secondary or primary amines or acids that interfere with the halo methylboronic acid pinacol ester tertiary amine reaction in the synthesis of the compounds, e.g., step e in FIG. 3C.

In embodiments, a PSMA targeting agent includes a moiety of

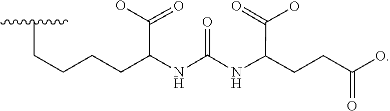

In embodiments, a fibroblast activation protein (FAP) inhibitor includes a moiety of

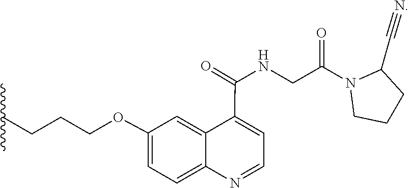 [SMILES: *CCCOc1ccc2nccc(C(=O)NCC(=O)N3CCCC3C#N)c2c1]

In embodiments, fibronectin or integrin targeted agent includes a moiety of

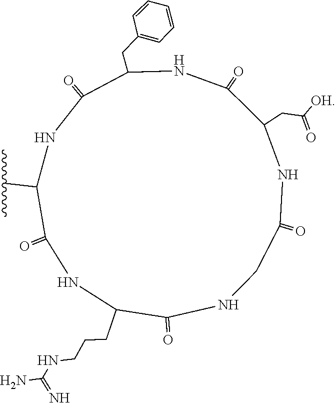 [SMILES: *C1NC(=O)C(Cc2ccccc2)NC(=O)C(CC(=O)O)NC(=O)CNC(=O)C(CCCNC(=N)N)NC1=O]

In embodiments, a Pentixafor chemokine receptor includes a moiety of

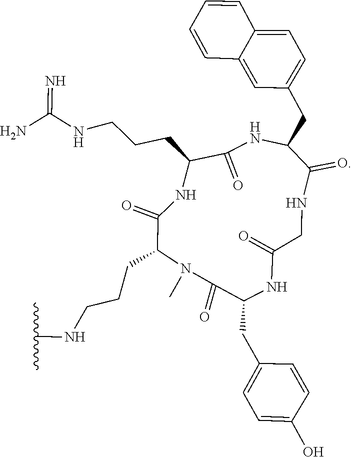 [SMILES: *NCCC[C@@H]1C(=O)N[C@@H](CCCNC(=N)N)C(=O)N[C@@H](Cc2ccc3ccccc3c2)C(=O)NCC(=O)N[C@H](Cc2ccc(O)cc2)C(=O)N1C]

In embodiments, a somatostatin targeted peptide (e.g., SST includes a moiety of

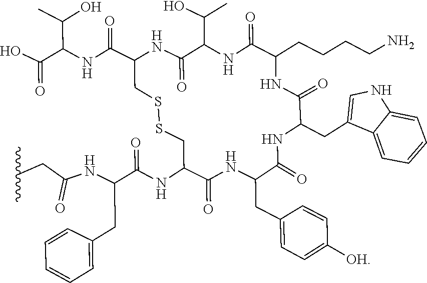 [SMILES: *CC(=O)NC(Cc1ccccc1)C(=O)NC1CSSCC(C(=O)NC(C(=O)O)C(C)O)NC(=O)C(C(C)O)NC(=O)C(CCCCN)NC(=O)C(Cc2c[nH]c3ccccc23)NC(=O)C(Cc2ccc(O)cc2)NC1=O]

In embodiments, the BT is

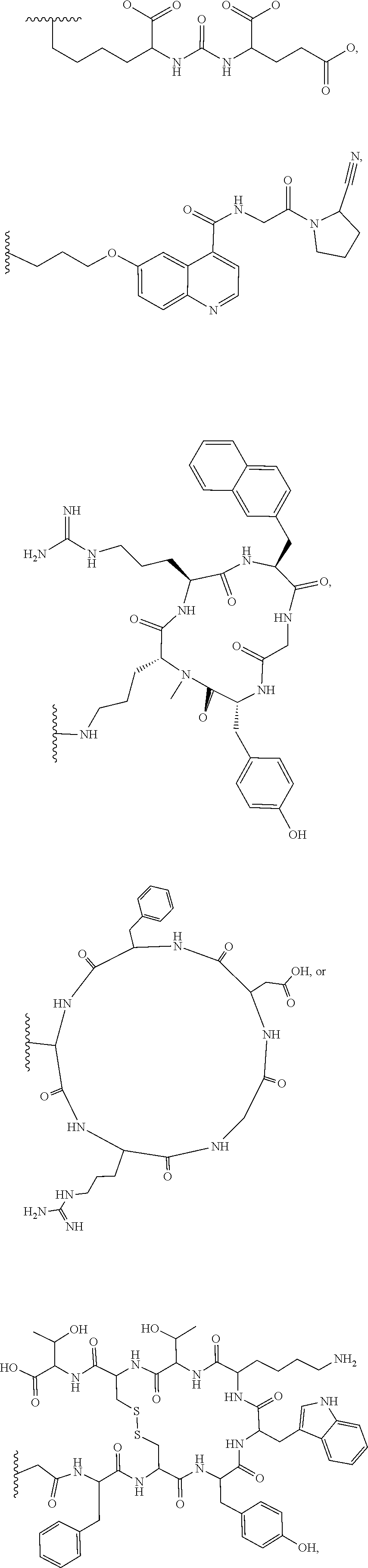

or pharmaceutically acceptable salt thereof.

In embodiments, the CL comprises one or more acyclic or macrocyclic derivatives containing ethylene diamine, amino ethyl thiol or hexadentate ligands. In embodiments, the CL comprises one or more of dodecane tetraacetic acid (DOTA), nitro-ROTA. 4-aminophenylethyl-1,4,7,10-tetraazacyclodecane-N,N,N″,N″-tetraacetic acid (PA-DOTA), diethylenetriaminepentaacetic acid (DTPA), (2-[4,7-bis(carboxymethyl)-1,4,7-triazonan-1-yl]acetic acid) NOTA, (triethylenetetramine) TETA, desferrioxamine, (ethylenediaminetetraacetic acid) EDTA, and penicillamine, or pharmaceutically acceptable salt thereof.

In embodiments, the compound may have a structure of Formula (I):

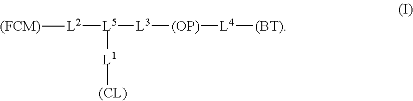

FCM, CL, OP, BT, L¹, L², L³, L⁴ and L⁵ are as described above.

In embodiments, L⁵ is

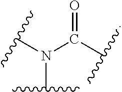

In embodiments, the compound may have a structure of Formula (I-a):

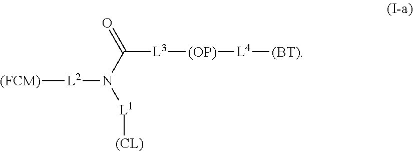

FCM, CL, OP, BT, L¹, L², L³, and L⁴ are described above.

In embodiments, in Formula (I) or (I-a), L¹ is L^{1A}-L^{1B}-L^{1C}-. In embodiments, L^{1A} is unsubstituted C₁-C₁₂ alkylene or oxo- or thio-substituted or unsubstituted 2 to 12 membered heteroalkylene. In embodiments, L^{1B} is unsubstituted phenylene. In embodiments, L^{1C} is unsubstituted C₁-C₁₂ alkylene, or oxo- or thio-substituted or unsubstituted 2 to 12 membered heteroalkylene.

In embodiments, L^{1A} is unsubstituted C₁-C₁₂ alkylene, L^{1B} is unsubstituted phenylene, and L^{1C} is oxo- or thio-substituted or unsubstituted 2 to 12 membered heteroalkylene. In embodiments, L^{1A} is methylene. In embodiments, L^{1A} is oxo- or thio-substituted or unsubstituted 2 to 12 membered heteroalkylene, L^{1B} is unsubstituted phenylene, and L^{1c} is unsubstituted C₁-C₁₂ alkylene. In embodiments, L^{2C} is methylene. In embodiments, L¹ is

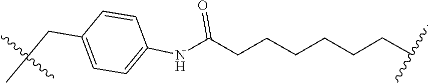

In embodiments, L¹ is -L^{1A}-C(O)NR¹¹L^{1B}-, or -L^{1A}NR¹¹C(O)L^{1B}-. In embodiments, L^{1A} and L^{1B} is independently unsubstituted C₁-C₁₂ alkylene, or unsubstituted phenylene. In embodiments, L^{1A} unsubstituted C₁-C₁₂ alkylene and L^{1B} is unsubstituted phenylene. In embodiments, L^{1A} unsubstituted phenylene and L^{1B} is unsubstituted C₁-C₁₂ alkylene. In embodiments, R¹¹ is a hydrogen. In embodiments, L¹ is

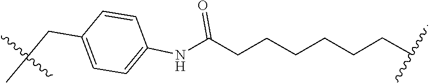

In embodiments, L² is unsubstituted C₁-C₁₂ alkylene. In embodiments, L³ is unsubstituted C₁-C₁₂ alkylene. In embodiments, each L² and L³ is independently unsubstituted C₁-C₁₂ alkylene.

In embodiments, L⁴ is unsubstituted C₁-C₁₂ alkylene or -L^{4A}NC(O)L^{4B}-. In embodiments, L^{4A} and L^{4B} is independently a bond, or unsubstituted C₁-C₁₂ alkylene. In embodiments, L^{4A} is a bond, and L^{4B} is unsubstituted C₁-C₁₂ alkylene. In embodiments, L^{4A} is unsubstituted C₁-C₁₂ alkylene and L^{4B} is a bond.

In embodiments, the compound may have a structure of Formula (I-a-1):

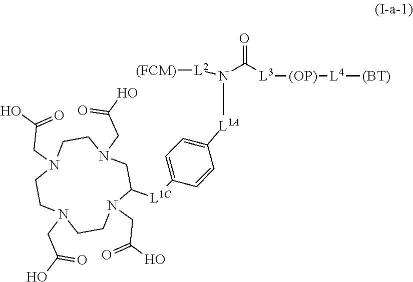

FCM, OP, BT, L^{1A}, L^{1C}, L², L³, and L⁴ are described above.

In embodiments, L^{1A} is oxo-substituted or unsubstituted 2 to 12 membered heteroalkylene. In embodiments, L^{1C} is unsubstituted C₁-C₁₂ alkylene.

In embodiments, L^{1A} is

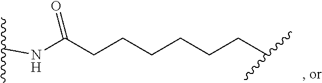 [SMILES: *CCCCCCC(=O)N*]

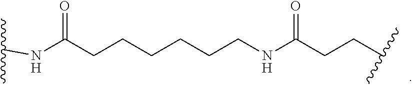 [SMILES: *CCC(=O)NCCCCCCC(=O)N*]

In embodiments, L^{1C} is unsubstituted methylene. In embodiments, L^{1C} is unsubstituted ethylene.

In embodiments, the compound is

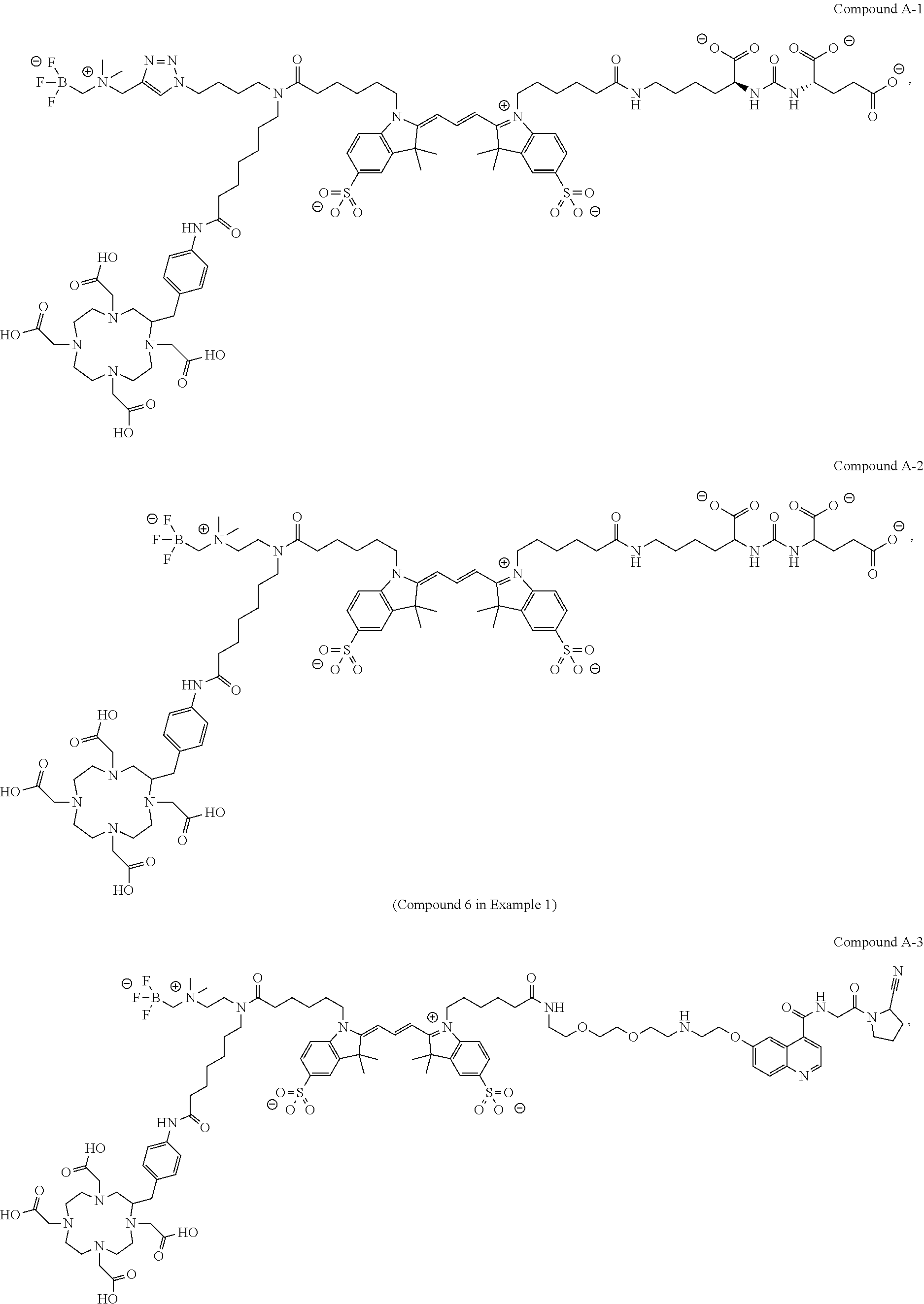

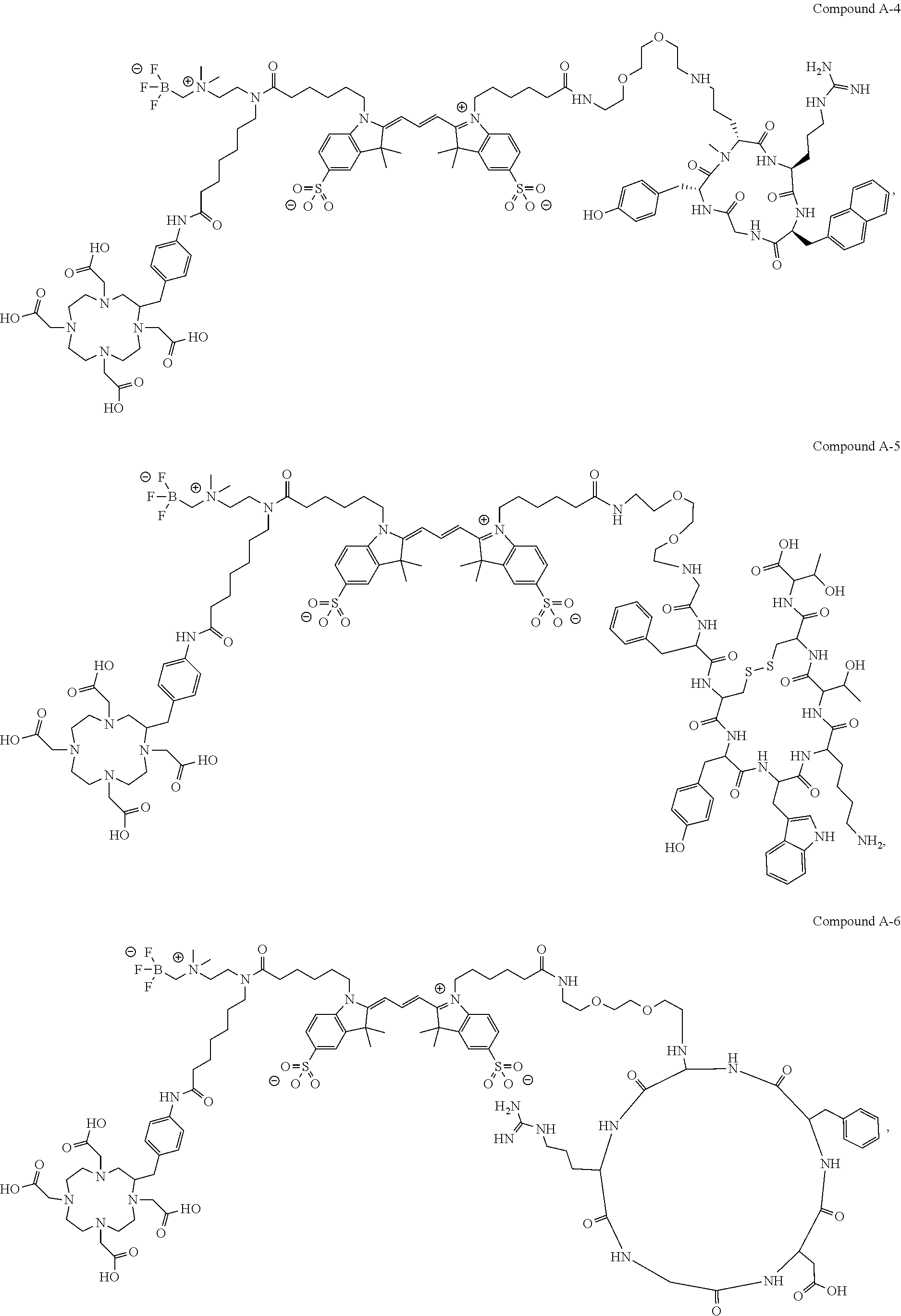

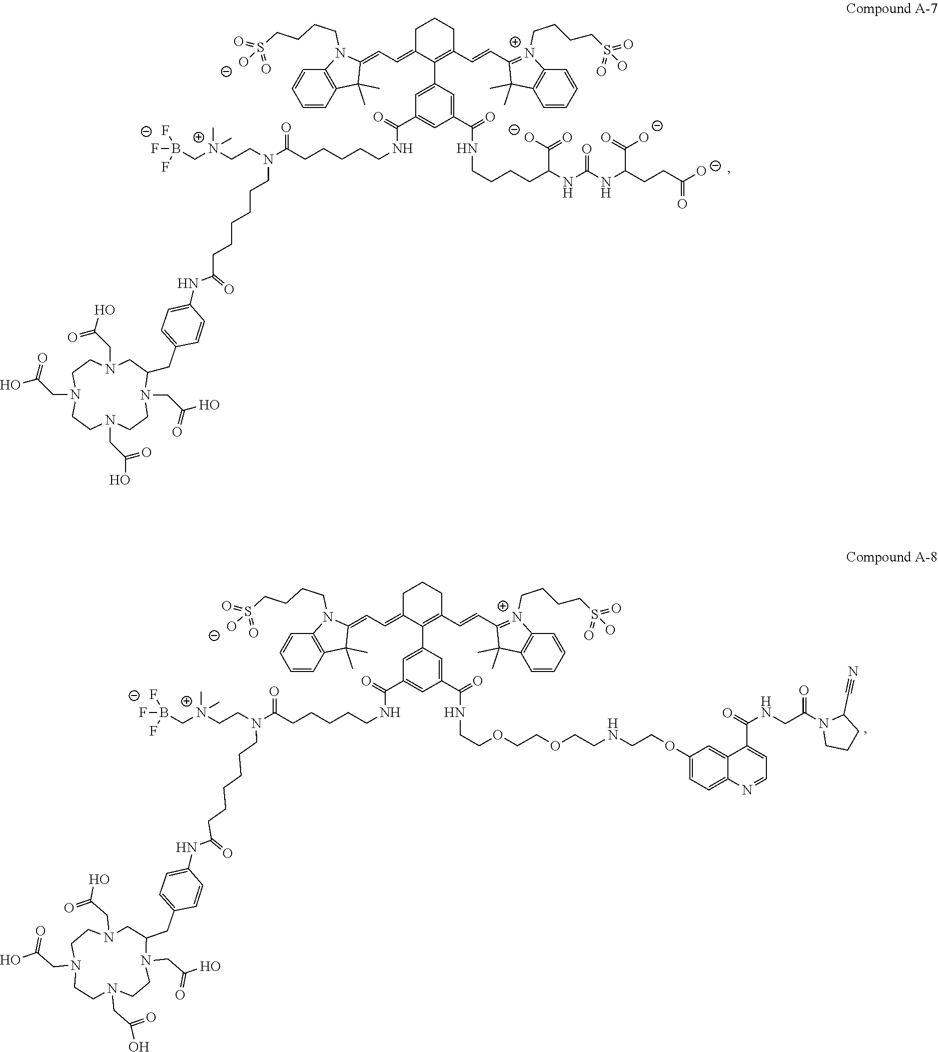

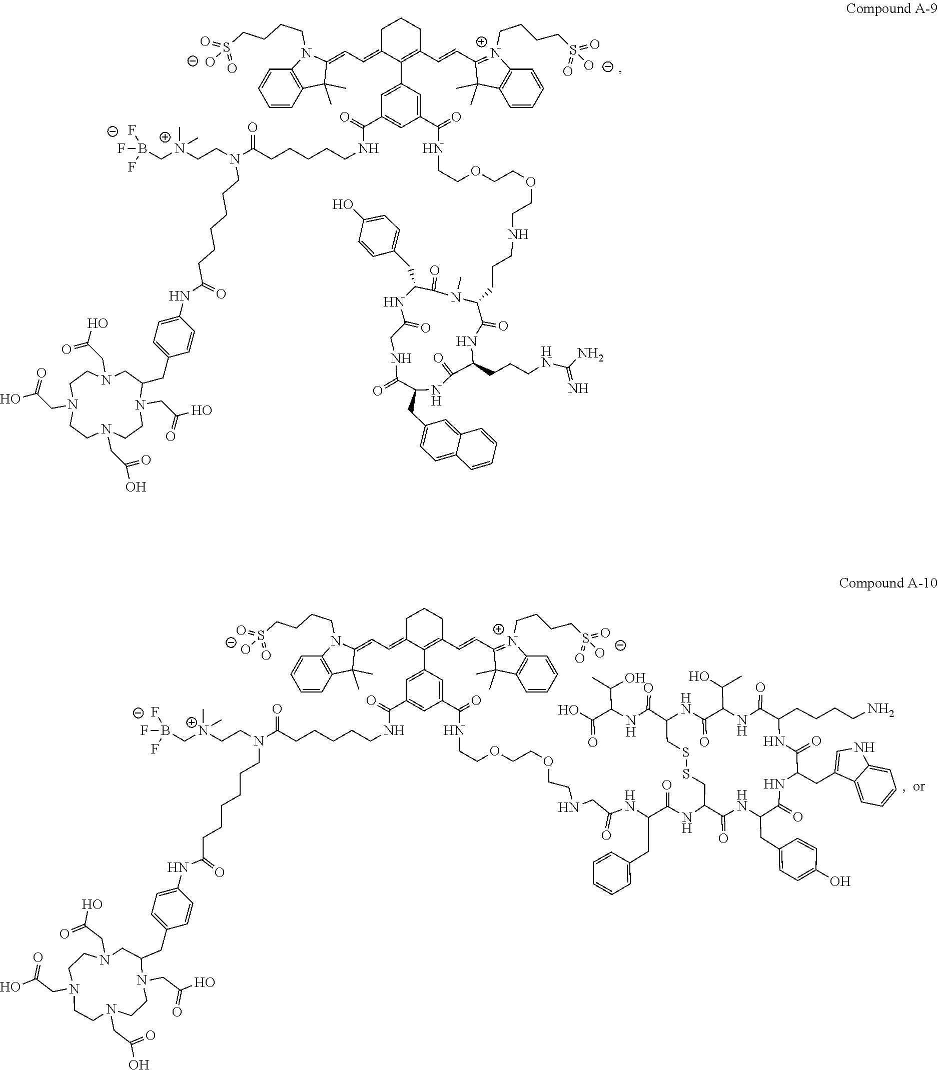

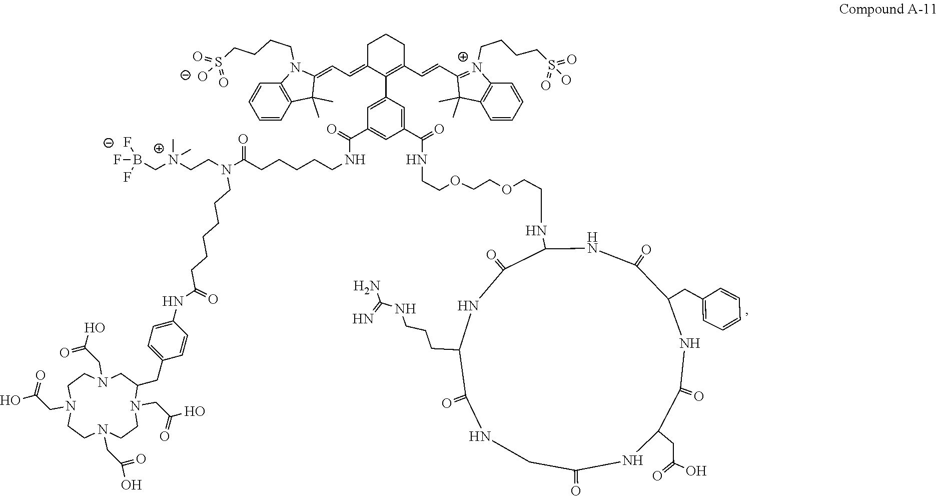

or a pharmaceutically acceptable salt thereof.

In embodiments, the compound may have a structure of Formula (II):

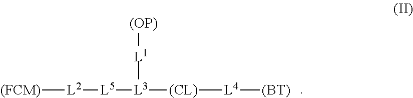

FCM, CL, OP, BT, L¹, L², L³, L⁴ and L⁵ are described above.

In embodiments, L¹ is a bond, or unsubstituted C₁-C₁₂ alkylene. In embodiments, L¹ is a bond. In embodiments, L¹ is methylene.

In embodiments, L⁵ is

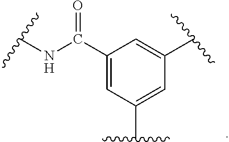 [SMILES: *NC(=O)c1cc(*)cc(*)c1]

In embodiments, L² is -L^{2A}-L^{2B}-L^{2C}-. In embodiments, each L^{2A}, L^{2B}, and L^{2C} is independently a bond, unsubstituted Ci-Cu alkylene, or oxo- or thio-substituted 5 to 10 membered heteroalkyl. In embodiments, L^{2A} is a bond, unsubstituted C₁-C₁₂ alkylene, or oxo- or thio-substituted 5 to 10 membered heteroalkyl. In embodiments, L^{2B} is a bond, unsubstituted C₁-C₁₂ alkylene, or oxo- or thio-substituted 5 to 10 membered heteroalkyl. In embodiments, L^{2C} is a bond, unsubstituted C₁-C₁₂ alkylene, or oxo- or thio-substituted 5 to 10 membered heteroalkyl.

In embodiments, L³ is -L^{3A}-L^{3B}-L^{3C}-. In embodiments, L^{3A} is unsubstituted C₁-C₁₂ alkylene or oxo- or thio-substituted or unsubstituted 2 to 12 membered heteroalkylene. In embodiments, L^{3B} is unsubstituted phenylene. In embodiments, L^{3C} is unsubstituted C₁-C₁₂ alkylene, or oxo- or thio-substituted or unsubstituted 2 to 12 membered heteroalkylene.

In embodiments, L^{3A} is unsubstituted C₁-C₁₂ alkylene, L^{3B} is unsubstituted phenylene, and L^{3C} is oxo- or thio-substituted or unsubstituted 2 to 12 membered heteroalkylene. In embodiments, L^{3A} is methylene. In embodiments, L^{3A} is oxo- or thio-substituted or unsubstituted 2 to 12 membered heteroalkylene, L^{3B} is unsubstituted phenylene, and L^{3C} is unsubstituted C₁-C₁₂ alkylene. In embodiments, L^{3C} is methylene.

In embodiments, L⁴ is -L^{4A}-L^{4B}-L^{4C}. In embodiments, L^{4A} is unsubstituted C₁-C₁₂ alkylene or oxo- or thio-substituted or unsubstituted 2 to 12 membered heteroalkylene. In embodiments, L^{4B} is unsubstituted phenylene. In embodiments, L^{4C} is unsubstituted C₁-C₁₂ alkylene, or oxo- or thio-substituted or unsubstituted 2 to 12 membered heteroalkylene.

In embodiments, L^{4A} is unsubstituted C₁-C₁₂ alkylene, L^{4B} is unsubstituted phenylene, and L^{4C} is oxo- or thio-substituted or unsubstituted 2 to 12 membered heteroalkylene. In embodiments, L^{4A} is methylene. In embodiments, L^{4A} is oxo- or thio-substituted or unsubstituted 2 to 12 membered heteroalkylene, L^{4B} is unsubstituted phenylene, and L^{4C} is unsubstituted C₁-C₁₂ alkylene. In embodiments, L^{4C} is methylene.

In embodiments, the compound is

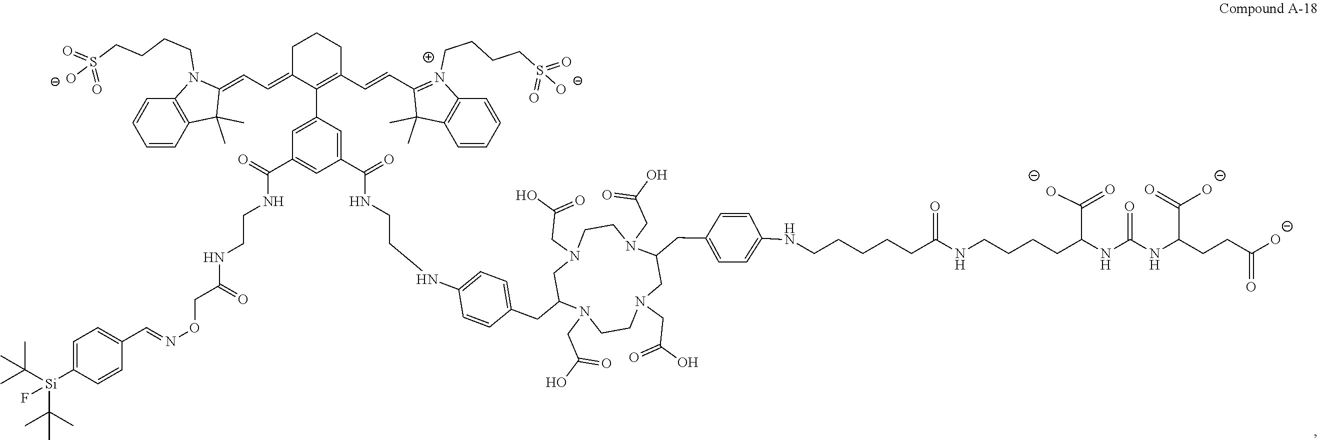

or pharmaceutically acceptable salt thereof.

In embodiments, the compound may have a structure of:

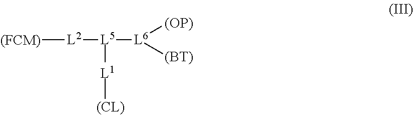

FCM, CL, OP, BT, L¹, L², L⁵ and L⁶ are described above.

In embodiments, in Formula (III), L¹ is -L^{1A}-L^{1B}-L^{1C}-. In embodiments, L^{1A} is unsubstituted C₁-C₁₂ alkylene or oxo- or thio-substituted or unsubstituted 2 to 12 membered heteroalkylene. In embodiments, L^{1B} is unsubstituted phenylene. In embodiments, L^{1C} is unsubstituted C₁-C₁₂ alkylene, or oxo- or thio-substituted or unsubstituted 2 to 12 membered heteroalkylene.

In embodiments, L^{1A} is unsubstituted C₁-C₁₂ alkylene, L^{1B} is unsubstituted phenylene, and L^{1C} is oxo- or thio-substituted or unsubstituted 2 to 12 membered heteroalkylene. In embodiments, L^{1A} is methylene. In embodiments, L^{1A} is oxo- or thio-substituted or unsubstituted 2 to 12 membered heteroalkylene, L^{1B} is unsubstituted phenylene, and L^{1C} is unsubstituted C₁-C₁₂ alkylene. In embodiments, L^{1C} is methylene. In embodiments, L¹ is

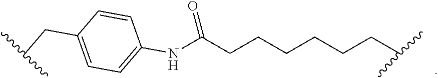 [SMILES: *CCCCCCC(=O)Nc1ccc(C*)cc1]

In embodiments, L¹ is L^{1A}C(O)NR¹¹L^{1B}-, or -L^{1A}NR¹¹C(O)L^{1B}-. In embodiments, L^{1A} and L^{1B} is independently unsubstituted C₁-C₁₂ alkylene, or unsubstituted phenylene. In embodiments, L^{1A} unsubstituted C₁-C₁₂ alkylene and L^{1B} is unsubstituted phenylene. In embodiments, L^{1A} unsubstituted phenylene and L^{1B} is unsubstituted C₁-C₁₂ alkylene. In embodiments, R¹¹ is a hydrogen. In embodiments, L¹ is

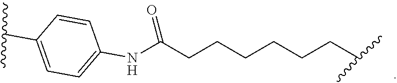 [SMILES: *CCCCCCC(=O)Nc1ccc(*)cc1]

In embodiments, L² is unsubstituted C₁-C₁₂ alkylene.

In embodiments, L⁵ is

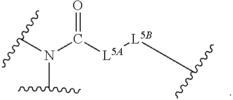

In embodiments, L^{5A} and L^{5B} is independently a bond, unsubstituted C₁-C₁₂ alkylene, or oxo- or thio-substituted 5 to 10 membered heteroalkyl. In embodiments, L^{5A} is a bond, unsubstituted C₁-C₁₂ alkylene, or oxo- or thio-substituted 5 to 10 membered heteroalkyl. In embodiments, L^{5B} is a bond, unsubstituted C₁-C₁₂ alkylene, or oxo- or thio-substituted 5 to 10 membered heteroalkyl.

In embodiments, L⁵ is

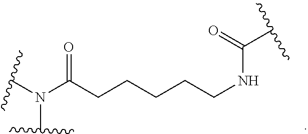 [SMILES: *C(=O)NCCCCCC(=O)N(*)*]

In embodiments, L⁶ is

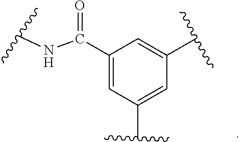 [SMILES: *NC(=O)c1cc(*)cc(*)c1]

In embodiments, L⁵-L⁶ is

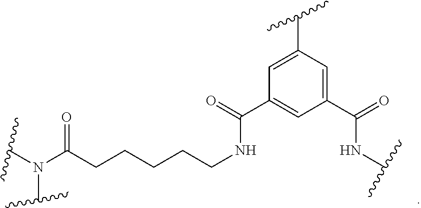 [SMILES: *NC(=O)c1cc(*)cc(C(=O)NCCCCCC(=O)N(*)*)c1]

In embodiments, the compound is

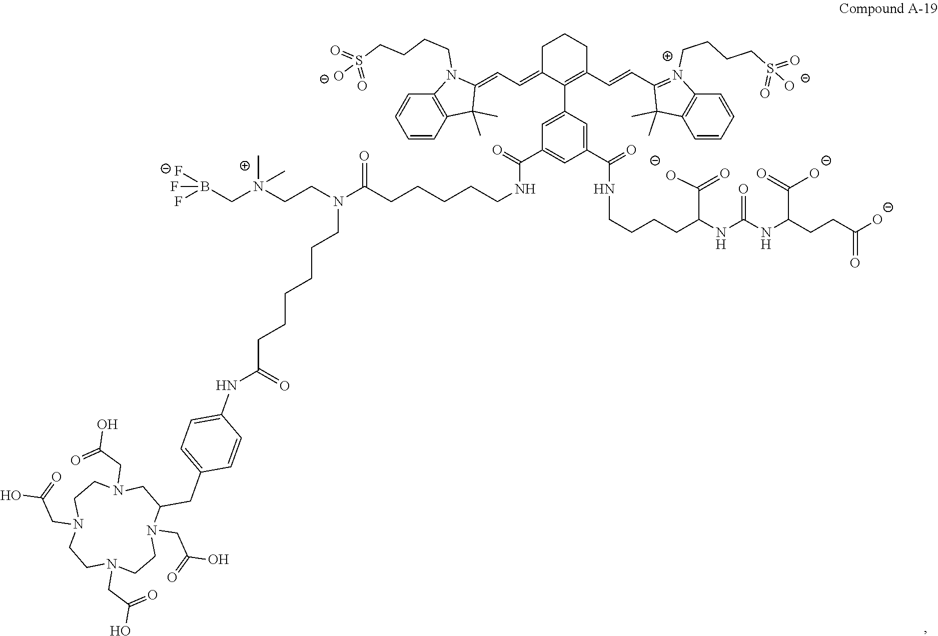

or pharmaceutically acceptable salt thereof.

In embodiments, the compound may have a structure of:

(FCM)-L²-(OP)-L³-(CL)-L⁴-(BT) (IV-a), or

(FCM)-L²-(CL)-L³-(OP)-L⁴-(BT) (IV-b).

FCM, CL, OP, BT, L², L³, L⁴ and L⁵ are described above.

In embodiments, in Formula (IV-a) or (IV-b), L² is -L^{2A}-L^{2A}-L^{2B}-L^{2C}-. In embodiments, L^{2A} is unsubstituted C₁-C₁₂ alkylene or oxo- or thio-substituted or unsubstituted 2 to 12 membered heteroalkylene. In embodiments, L^{2B} is unsubstituted phenylene. In embodiments, L^{2C} is unsubstituted C₁-C₁₂ alkylene, or oxo- or thio-substituted or unsubstituted 2 to 12 membered heteroalkylene.

In embodiments, L^{2A} is unsubstituted C₁-C₁₂ alkylene, L^{2B} is unsubstituted phenylene, and L^{2C} is oxo- or thio-substituted or unsubstituted 2 to 12 membered heteroalkylene. In embodiments, L^{2A} is methylene. In embodiments, L^{2A} is oxo- or thio-substituted or unsubstituted 2 to 12 membered heteroalkylene, L^{2B} is unsubstituted phenylene, and L^{2C} is unsubstituted C₁-C₁₂ alkylene. In embodiments, L^{2C} is methylene. In embodiments, L² is

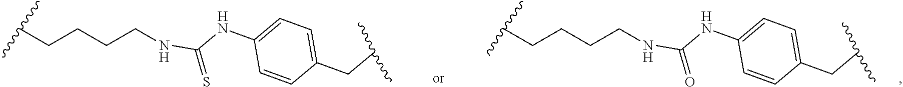 [SMILES: *CCCCNC(=S)Nc1ccc(C*)cc1 ; *CCCCNC(=O)Nc1ccc(C*)cc1]

In embodiments, in Formula (IV-a) or (IV-b), L³ is -L^{3A}-L^{3B}-L^{3C}-. In embodiments, L^{3A} is unsubstituted C₁-C₁₂ alkylene or oxo- or thio-substituted or unsubstituted 2 to 12 membered heteroalkylene. In embodiments, L^{3B} is unsubstituted phenylene. In embodiments, L^{3C} is unsubstituted C₁-C₁₂ alkylene, or oxo- or thio-substituted or unsubstituted 2 to 12 membered heteroalkylene.

In embodiments, L^{3A} is unsubstituted C₁-C₁₂ alkylene, L^{3B} is unsubstituted phenylene, and L^{3C} is oxo- or thio-substituted or unsubstituted 2 to 12 membered heteroalkylene. In embodiments, L^{3A} is methylene. In embodiments, L^{3A} is oxo- or thio-substituted or unsubstituted 2 to 12 membered heteroalkylene, L^{3B} is unsubstituted phenylene, and L^{3C} is unsubstituted C₁-C₁₂ alkylene. In embodiments, L^{3C} is methylene. In embodiments, L³ is

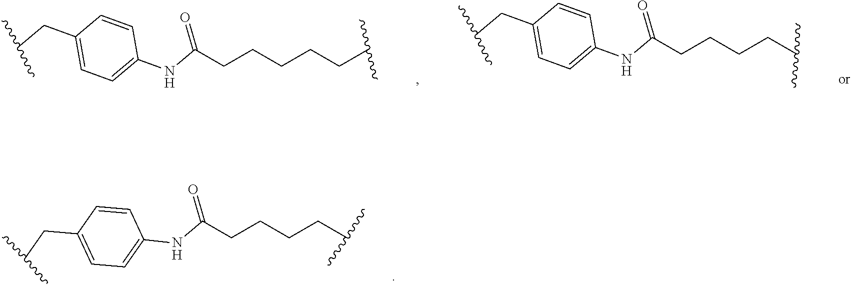 [SMILES: *CCCCCC(=O)Nc1ccc(C*)cc1 ; *CCCCC(=O)Nc1ccc(C*)cc1 ; *CCCCC(=O)Nc1ccc(C*)cc1]

In embodiments, L³ is -L^{3A}C(O)NR¹³L^{3B}-, or -L^{3A}NR¹³C(O)L^{3B}-. In embodiments, L^{3A} and L^{3B} is independently unsubstituted C₁-C₁₂ alkylene, or unsubstituted phenylene. In embodiments, L^{3A} unsubstituted C₁-C₁₂ alkylene and L^{3B} is unsubstituted phenylene. In embodiments, L^{3A} unsubstituted phenylene and L^{3B} is unsubstituted C₁-C₁₂ alkylene. In embodiments, R¹¹ is a hydrogen. In embodiments, L³ is

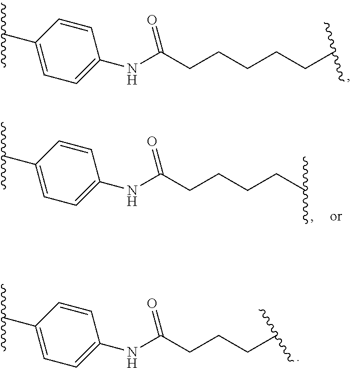 [SMILES: *CCCCCC(=O)Nc1ccc(*)cc1 ; *CCCCC(=O)Nc1ccc(*)cc1 ; *CCCC(=O)Nc1ccc(*)cc1]

In embodiments, L⁴ is -L^{4A}-L^{4B}, -L^{4A}C(O)NR¹⁴L^{4B}-, or -L^{4A}NR¹⁴C(O)L^{4B}-. In embodiments, each L^{4A} and L^{4B} is independently unsubstituted C₁-C₁₂ alkylene, or oxo- or thio-substituted or unsubstituted 2 to 12 membered heteroalkylene. In embodiments, L^{4A} is unsubstituted C₁-C₁₂ alkylene, or oxo- or thio-substituted or unsubstituted 2 to 12 membered heteroalkylene. In embodiments, L^{4B} is unsubstituted C₁-C₁₂ alkylene, or oxo- or thio-substituted or unsubstituted 2 to 12 membered heteroalkylene.

In embodiments, the compound is

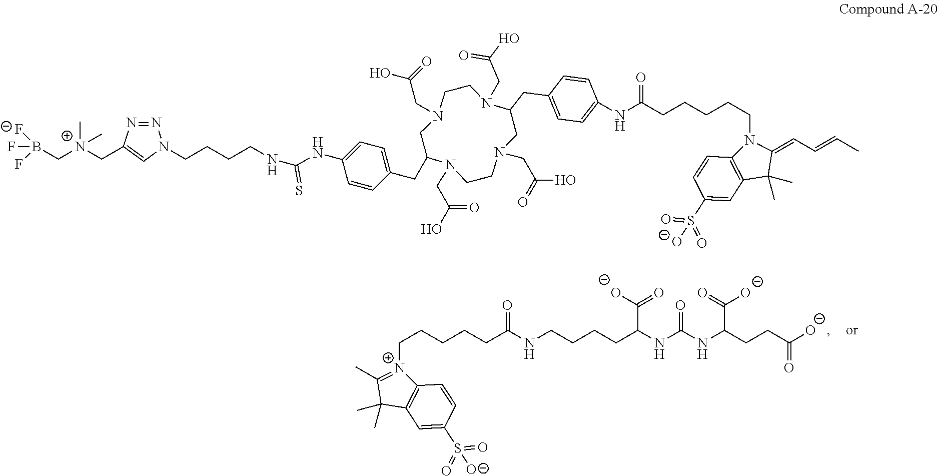

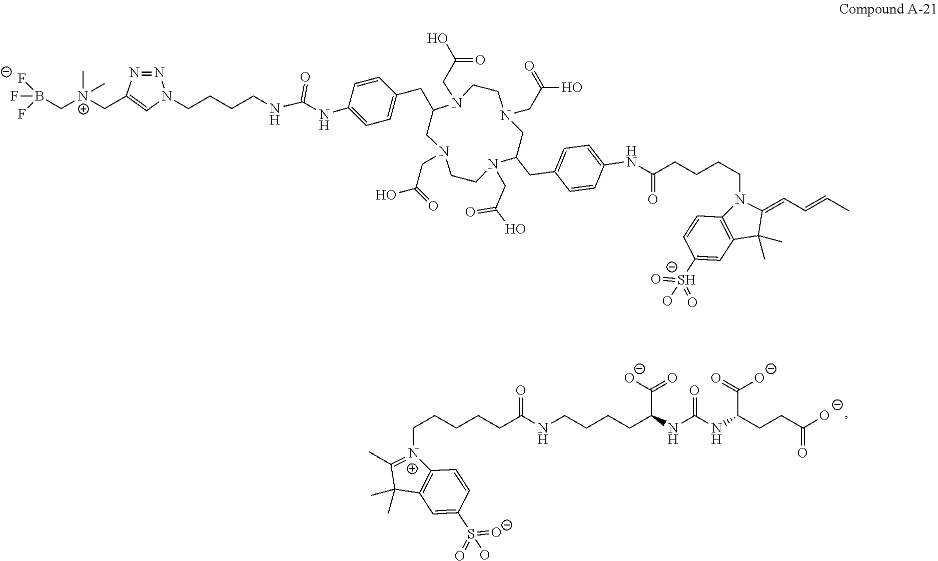

or pharmaceutically acceptable salt thereof.

In embodiments, the compound may have a structure of:

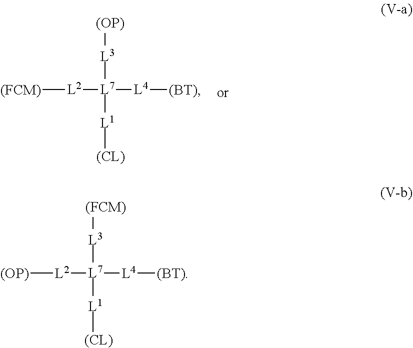

FCM, CL, OP, BT, L¹, L², L³, and L⁴ are described above.

In embodiments, the compound may have a structure of:

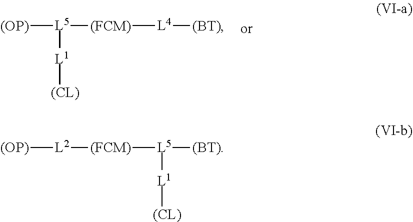

FCM, CL, OP, BT, L¹, L², L⁴, and L⁵ are described above.

In embodiments, in Formula (V), L⁷ is

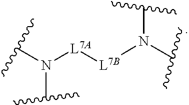

In embodiments, each L^{7A} and L^{7B} is independently a bond, unsubstituted C₁-C₁₂ alkylene, oxo- or thio-substituted or unsubstituted 2 to 12 membered heteroalkylene, or unsubstituted phenylene. In embodiments, L^{7A} is a bond, unsubstituted C₁-C₁₂ alkylene, oxo- or thio-substituted or unsubstituted 2 to 12 membered heteroalkylene, or unsubstituted phenylene. In embodiments, L^{7B} is a bond, unsubstituted C₁-C₁₂ alkylene, oxo- or thio-substituted or unsubstituted 2 to 12 membered heteroalkylene, or unsubstituted phenylene. In embodiments, L⁷ is

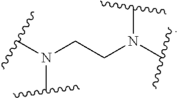 [SMILES: *N(*)CCN(*)*]

In embodiments, L¹ is -L^{1A}-L^{1B}-L^{1C}-. In embodiments, L^{1A} is unsubstituted C₁-C₁₂ alkylene or oxo- or thio-substituted or unsubstituted 2 to 12 membered heteroalkylene. In embodiments, L^{1B} is unsubstituted phenylene. In embodiments, L^{1C} is unsubstituted C₁-C₁₂ alkylene, or oxo- or thio-substituted or unsubstituted 2 to 12 membered heteroalkylene.

In embodiments, L^{1A} is unsubstituted C₁-C₁₂ alkylene, L^{1B} is unsubstituted phenylene, and L^{1C} is oxo- or thio-substituted or unsubstituted 2 to 12 membered heteroalkylene. In embodiments, L^{1A} is methylene. In embodiments, L^{1A} is oxo- or thio-substituted or unsubstituted 2 to 12 membered heteroalkylene, L^{1B} is unsubstituted phenylene, and L^{1C} is unsubstituted C₁-C₁₂ alkylene. In embodiments, L^{1C} is methylene. In embodiments, L¹ is

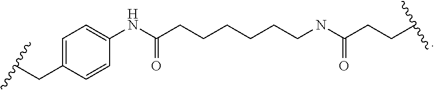 [SMILES: *CCC(=O)NCCCCCCC(=O)Nc1ccc(C*)cc1]

In embodiments, L² is -L^{2A}-L^{2B}-L^{2C}-. In embodiments, L³ is -L^{3A}-L^{3B}-L^{3C}-. In embodiments, L⁴ is -L^{4A}-L^{4B}-L^{4C}-. In embodiments, each L^{2A}, L^{2B}, L^{2C}, L^{3A}, L^{3B}, L^{3C}, L^{4A}, L^{4B}, and L^{4C} is independently a bond, unsubstituted C₁-C₁₂ alkylene, or oxo- or thio-substituted or unsubstituted 2 to 12 membered heteroalkylene.

In embodiments, the compound is

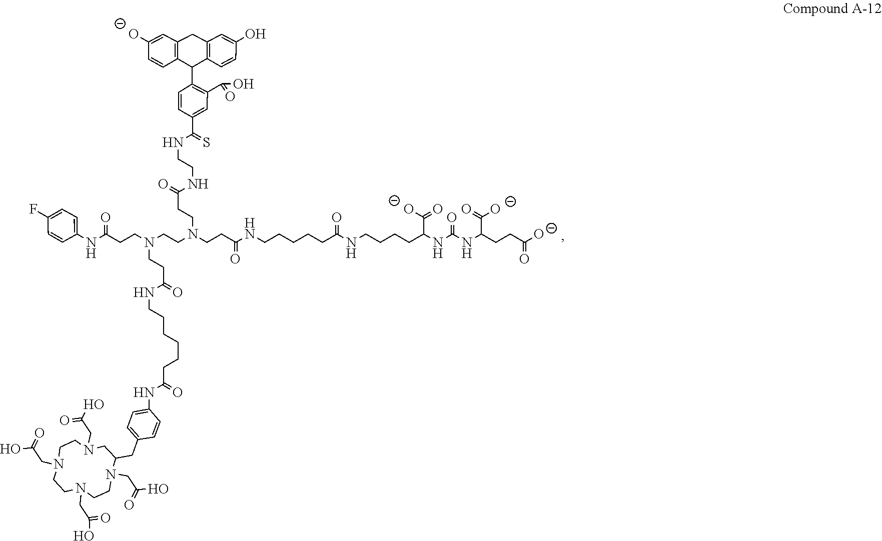 [SMILES: O=C([O-])CCC(NC(=O)NC(CCCCNC(=O)CCCCCNC(=O)CCN(CCC(=O)NCCNC(=S)c1ccc(C2c3ccc([O-])cc3Cc3cc(O)ccc32)c(C(=O)O)c1)CCN(CCC(=O)NCCCCCCC(=O)Nc1ccc(CC2CN(CC(=O)O)CCN(CC(=O)O)CCN(CC(=O)O)CCN2CC(=O)O)cc1)CCC(=O)Nc1ccc(F)cc1)C(=O)[O-])C(=O)[O-]]

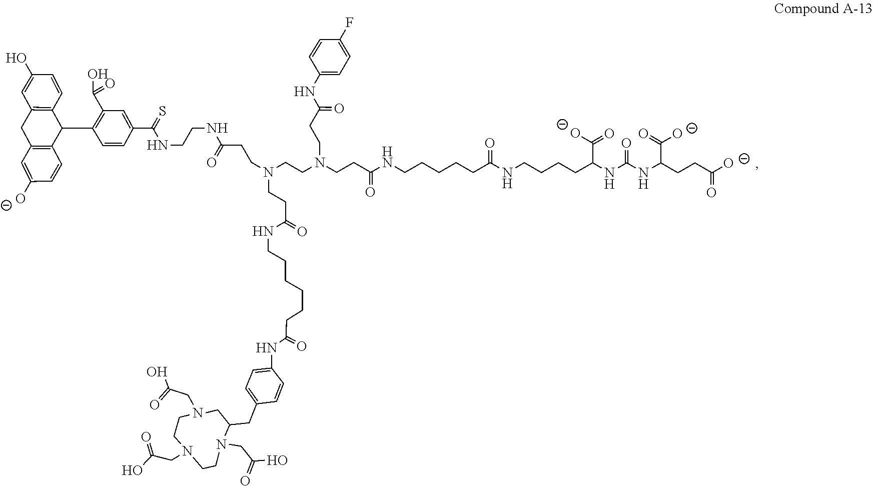 [SMILES: O=C([O-])CCC(NC(=O)NC(CCCCNC(=O)CCCCCNC(=O)CCN(CCC(=O)Nc1ccc(F)cc1)CCN(CCC(=O)NCCCCCCC(=O)Nc1ccc(CC2CN(CC(=O)O)CCN(CC(=O)O)CCN2CC(=O)O)cc1)CCC(=O)NCCNC(=S)c1ccc(C2c3ccc([O-])cc3Cc3cc(O)ccc32)c(C(=O)O)c1)C(=O)[O-])C(=O)[O-]]

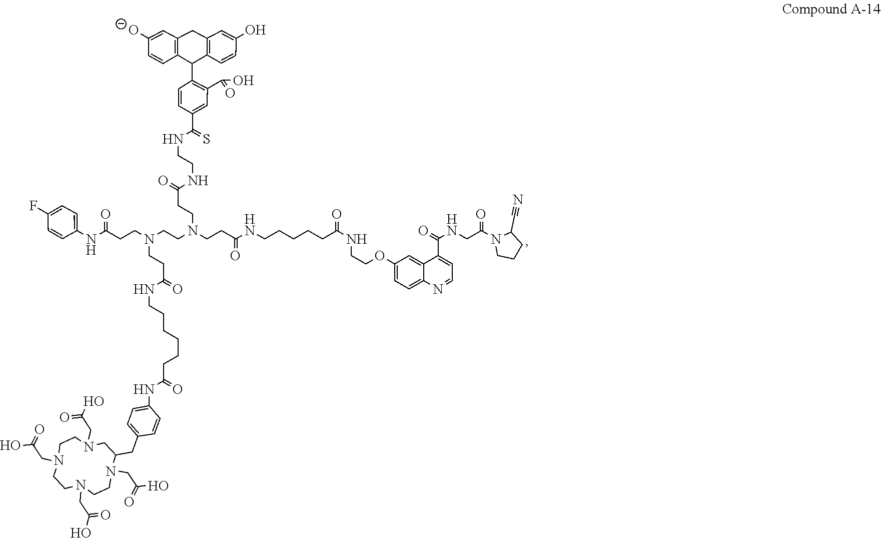 [SMILES: N#CC1CCCN1C(=O)CNC(=O)c1ccnc2ccc(OCCNC(=O)CCCCCNC(=O)CCN(CCC(=O)NCCNC(=S)c3ccc(C4c5ccc([O-])cc5Cc5cc(O)ccc54)c(C(=O)O)c3)CCN(CCC(=O)NCCCCCCC(=O)Nc3ccc(CC4CN(CC(=O)O)CCN(CC(=O)O)CCN(CC(=O)O)CCN4CC(=O)O)cc3)CCC(=O)Nc3ccc(F)cc3)cc12]

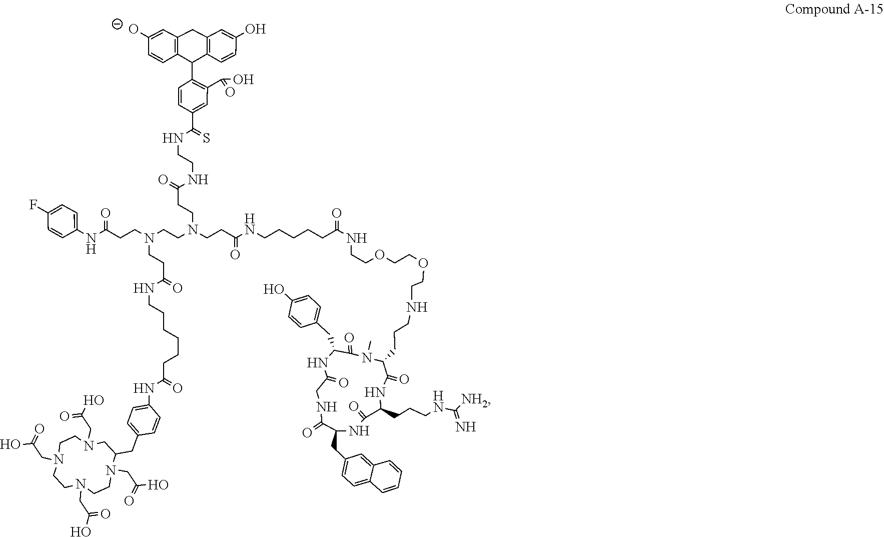 [SMILES: CN1C(=O)[C@@H](Cc2ccc(O)cc2)NC(=O)CNC(=O)[C@H](Cc2ccc3ccccc3c2)NC(=O)[C@H](CCCNC(=N)N)NC(=O)[C@H]1CCCNCCOCCOCCNC(=O)CCCCCNC(=O)CCN(CCC(=O)NCCNC(=S)c1ccc(C2c3ccc([O-])cc3Cc3cc(O)ccc32)c(C(=O)O)c1)CCN(CCC(=O)NCCCCCCC(=O)Nc1ccc(CC2CN(CC(=O)O)CCN(CC(=O)O)CCN(CC(=O)O)CCN2CC(=O)O)cc1)CCC(=O)Nc1ccc(F)cc1]

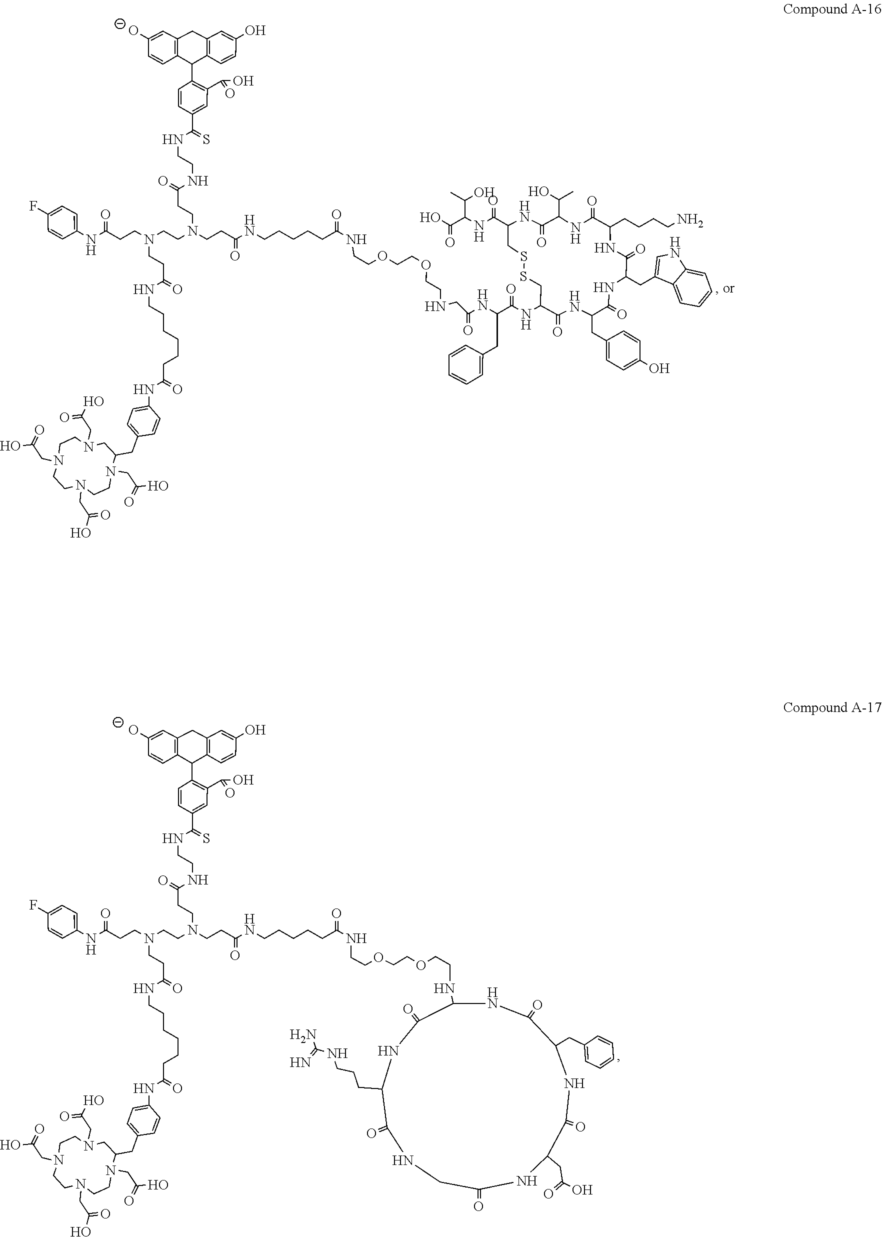

or pharmaceutically acceptable salt thereof.

In embodiments, the compound is

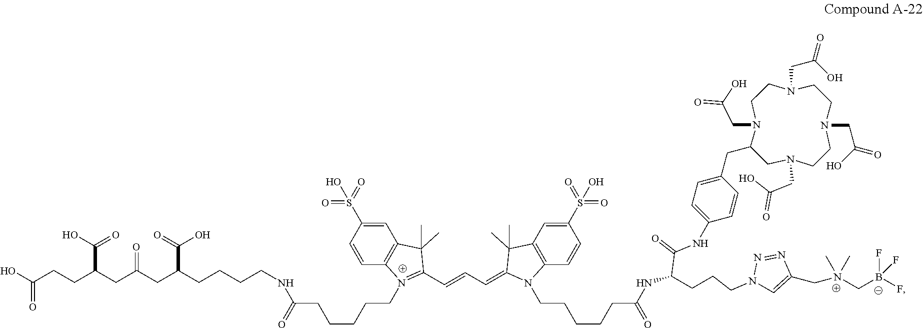

or pharmaceutically acceptable salt thereof.

In embodiments, exemplary compounds are summarized in the following Table 1.

TABLE 1

| | | | | Exemplary |
|---|---|---|---|---|
| FCM | CL | OP | BT | Compounds |
| 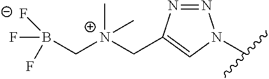 | DOTA 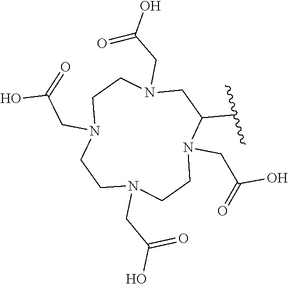 [SMILES: *C1CN(CC(=O)O)CCN(CC(=O)O)CCN(CC(=O)O)CCN1CC(=O)O] | cyanine-based fluorophores 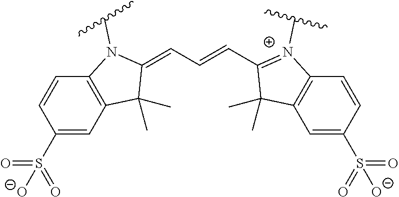 [SMILES: *N1/C(=C/C=C/C2=[N+](*)c3ccc(S(=O)(=O)[O-])cc3C2(C)C)C(C)(C)c2cc(S(=O)(=O)[O-])ccc21] | PSMA⁺ tumor | Compounds A-1, A-20, A-21, A-22 |
| 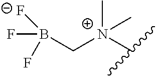 | DOTA 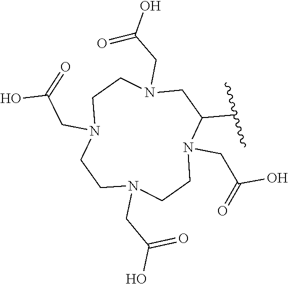 [SMILES: *C1CN(CC(=O)O)CCN(CC(=O)O)CCN(CC(=O)O)CCN1CC(=O)O] | cyanine-based fluorophores 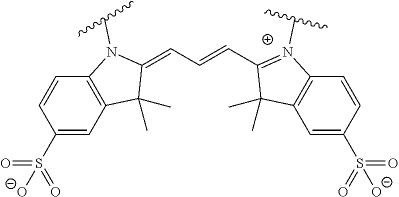 | PSMA⁺ tumor FAP marker CXCR marker SSTR marker Integrin marker | Compounds A-2, A-19 Compound A-3 Compound A-4 Compound A-5 Compound A-6 |
| | | 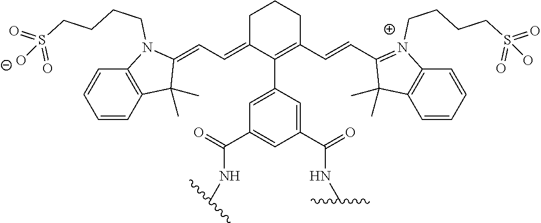 [SMILES: *NC(=O)c1cc(C(=O)N*)cc(C2=C(/C=C/C3=[N+](CCCCS(=O)(=O)O)c4ccccc4C3(C)C)CCC/C2=C\C=C2\N(CCCCS(=O)(=O)[O-])c3ccccc3C2(C)C)c1] | PSMA⁺ tumor FAP marker CXCR marker SSTR marker Integrin marker | Compound A-7 Compound A-8 Compound A-9 Compound A-10 Compound A-11 |
| 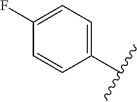 [SMILES: *c1ccc(F)cc1] | DOTA 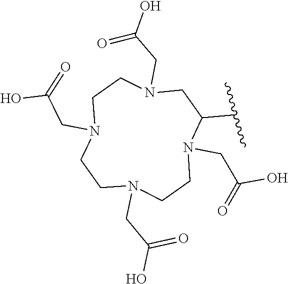 [SMILES: *C1CN(CC(=O)O)CCN(CC(=O)O)CCN(CC(=O)O)CCN1CC(=O)O] | 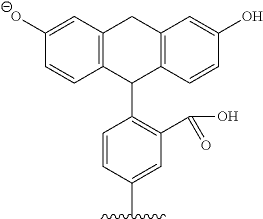 [SMILES: *c1ccc(C2c3ccc([O-])cc3Cc3cc(O)ccc32)c(C(=O)O)c1] | PSMA⁺ tumor FAP marker CXCR marker SSTR marker Integrin marker | Compound A-12 Compound A-14 Compound A-15 Compound A-16 Compound A-17 |
| 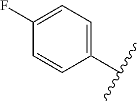 [SMILES: *c1ccc(F)cc1] | 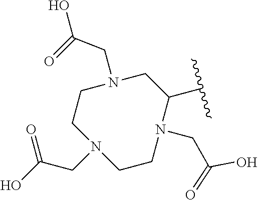 [SMILES: *C1CN(CC(=O)O)CCN(CC(=O)O)CCN1CC(=O)O] | 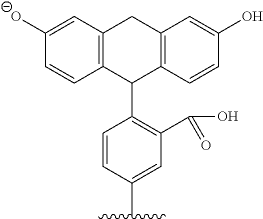 [SMILES: *c1ccc(C2c3ccc([O-])cc3Cc3cc(O)ccc32)c(C(=O)O)c1] | PSMA⁺ tumor | Compound A-13 |
| 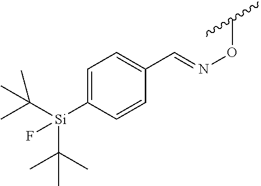 [SMILES: *O/N=C/c1ccc([Si](F)(C(C)(C)C)C(C)(C)C)cc1] | DOTA 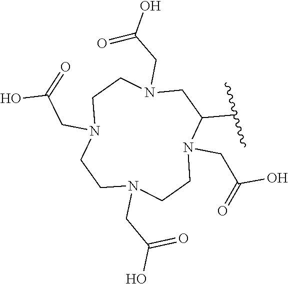 [SMILES: *C1CN(CC(=O)O)CCN(CC(=O)O)CCN(CC(=O)O)CCN1CC(=O)O] | 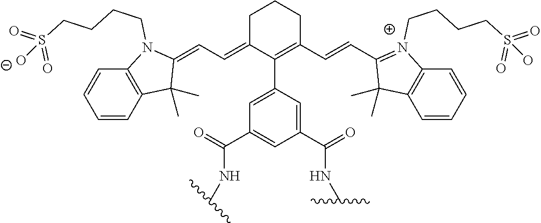 [SMILES: *NC(=O)c1cc(C(=O)N*)cc(C2=C(/C=C/C3=[N+](CCCCS(=O)(=O)O)c4ccccc4C3(C)C)CCC/C2=C\C=C2\N(CCCCS(=O)(=O)[O-])c3ccccc3C2(C)C)c1] | PSMA⁺ tumor | Compound A-18 |

In an aspect, provided is a compound including the following moieties:

(i) a chelating ligand moiety (CL);

(ii) an optical probe moiety (OP); and

(iii) a biological targeting moiety (BT),

wherein the compound has the structure of Formula (X).

In embodiments, the compound has a structure of Formula (X):

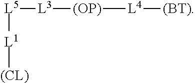

CL, OP, BT, L¹, L³, L⁴ and L⁵ are described above.

In some embodiments, the compound has a structure of Formula (X-a):

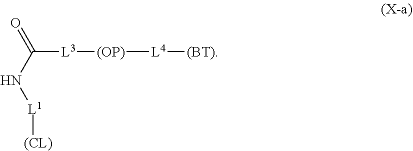

CL, OP, BT, L¹, L³, and L⁴ are described above.

In some embodiments, the compound has a structure of Formula (XI):

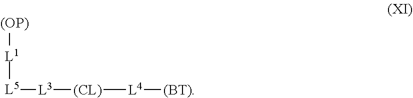

CL, OP, BT, L¹, L³, L⁴, and L⁵ are described above.

In some embodiments, the compound has a structure of Formula (XII):

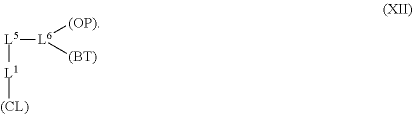

CL, OP, BT, L¹, L⁵, and L⁶ are described above.

In some embodiments, the compound has a structure of Formula (XIII-a) or (XIII-b):

(OP)-L³-(CL)-L⁴-(BT) (XIII-a), or

(CL)-L³-(OP)-L⁴-(BT) (XIII-b).

CL, OP, BT, L³, and L⁴ are described above.

In some embodiments, the compound is:

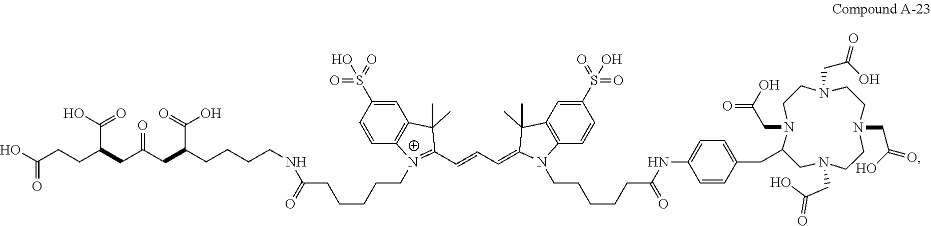

or pharmaceutically acceptable salt thereof.

In some embodiments, the compound has a structure of Formula (XIV-a) or (XIV-b):

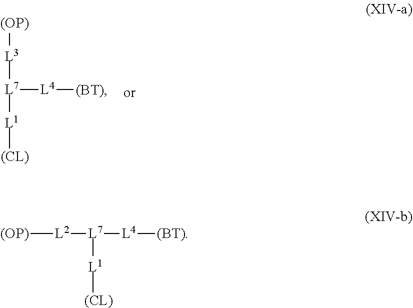

CL, OP, BT, L¹, L³, L⁴, and L⁷ are described above.

In another aspect, provided are precursors of the compounds described herein. In embodiments, the precursors include protection group (e.g., tert-Butyloxycarbonyl (BOC), 9-Fluorenylmethyloxycarbonyl (Fmoc), Acetyl (Ac), β-Methoxyethoxymethyl ether (MEM), Dimethoxytrityl, [bis-(4-methoxyphenyl)phenylmethyl](DMT), Carbobenzyloxy (Cbz), or p-Methoxybenzyl carbonyl (Moz or MeOZ)). In embodiments, the precursors are deprotected to produce the compounds described herein.

In embodiments, the precursor has the structure:

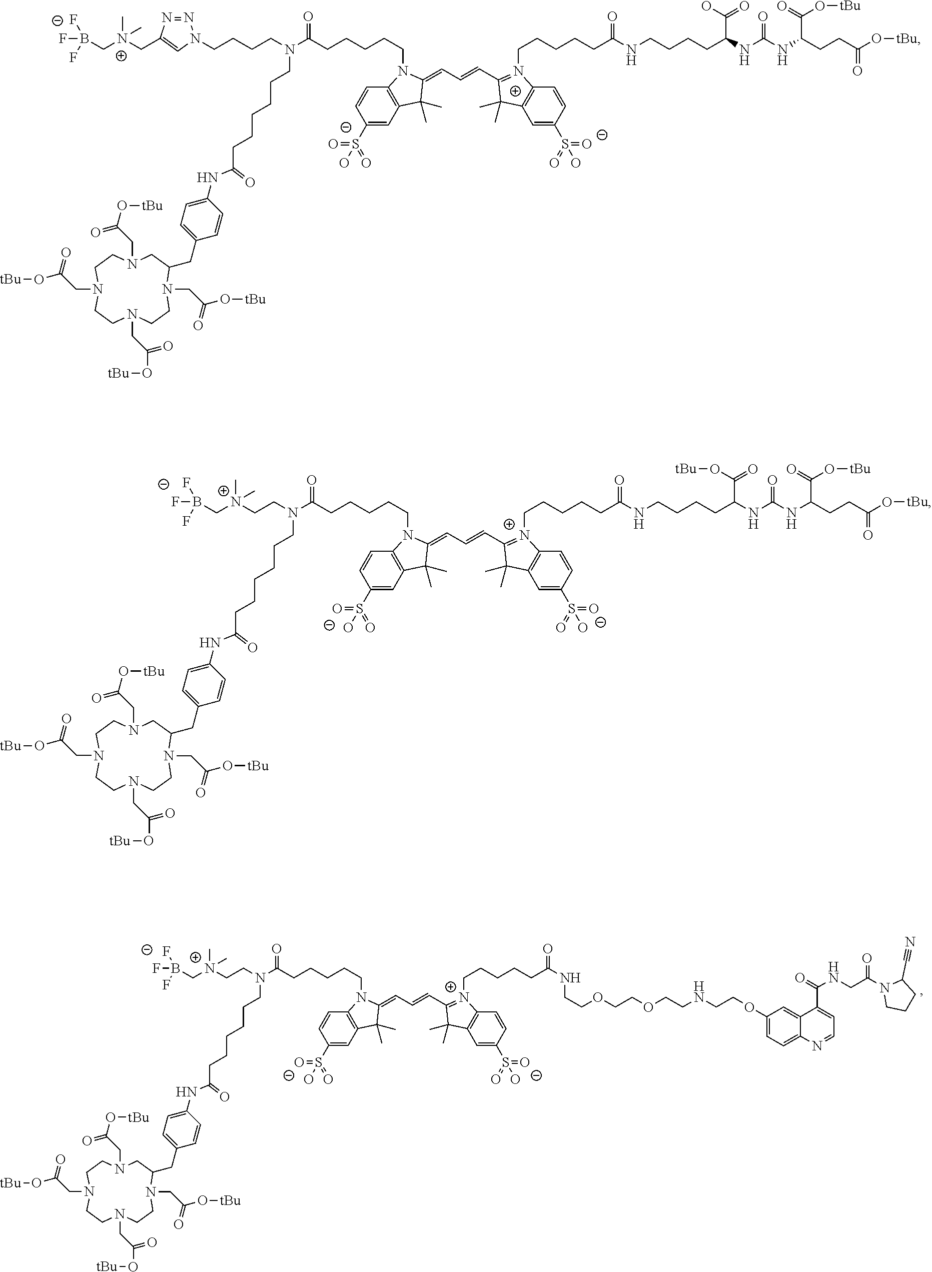

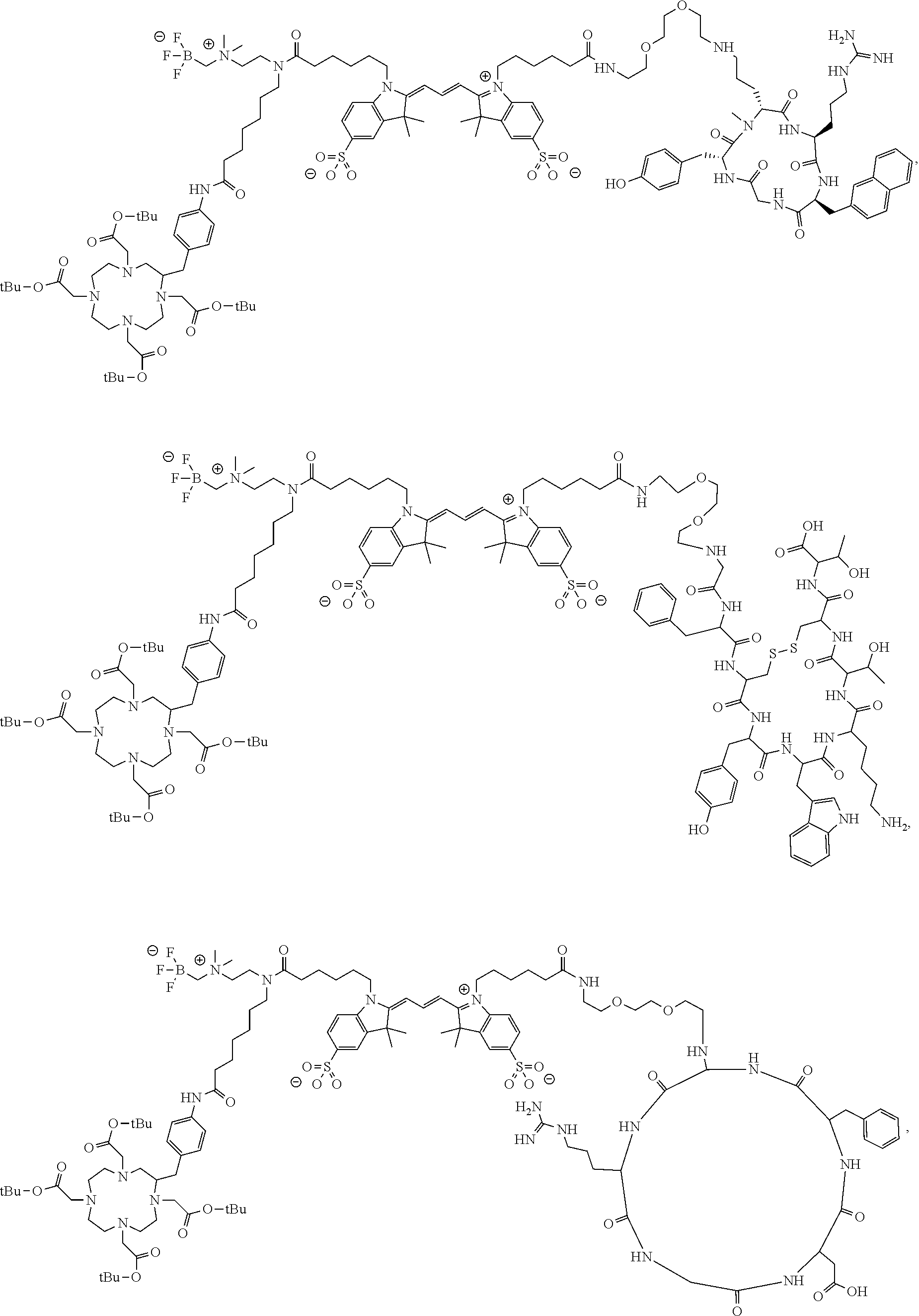

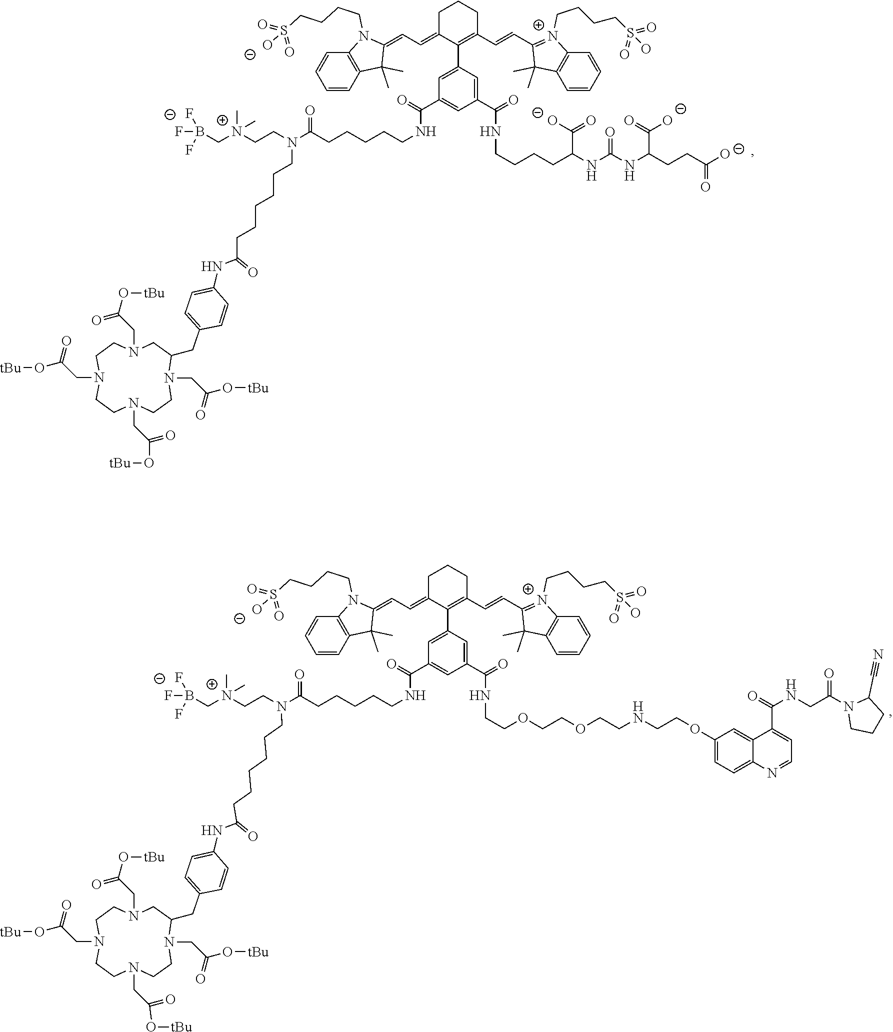

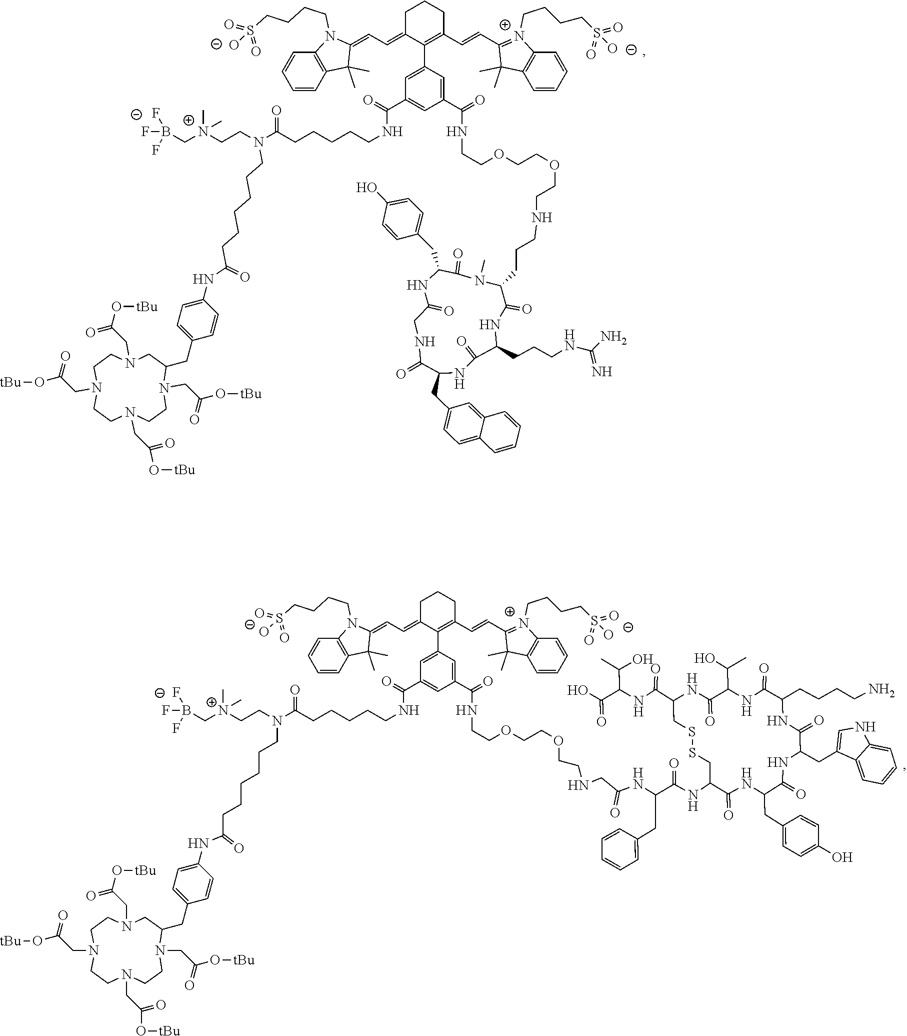

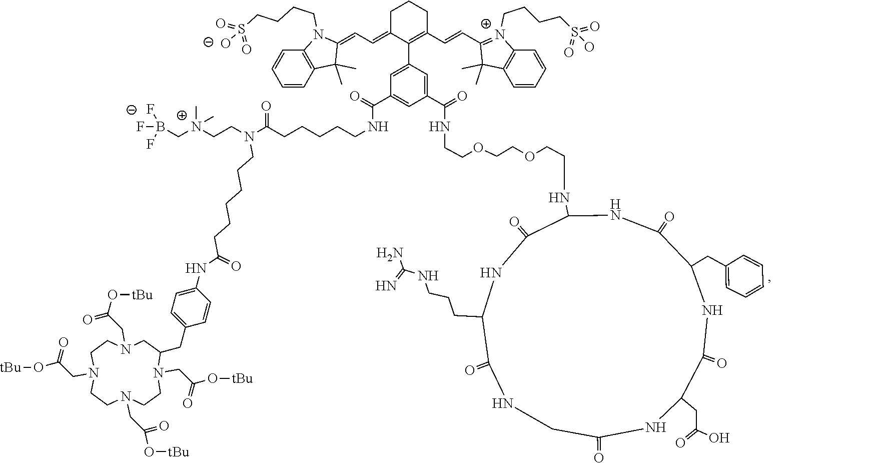

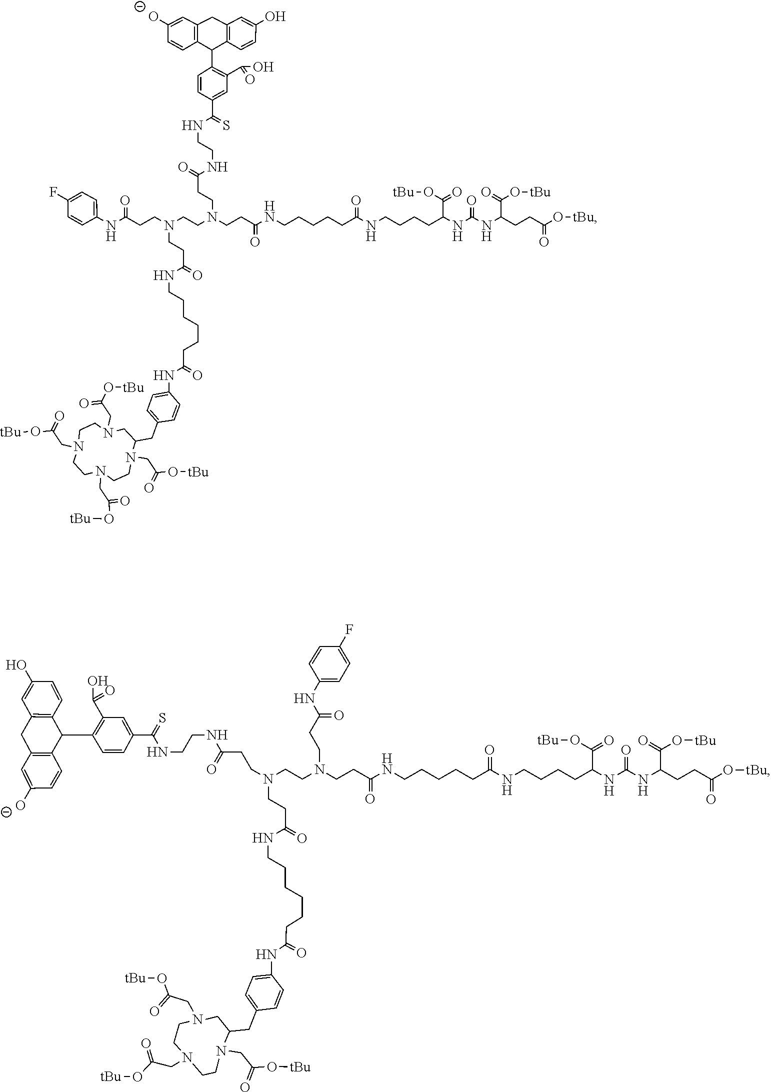

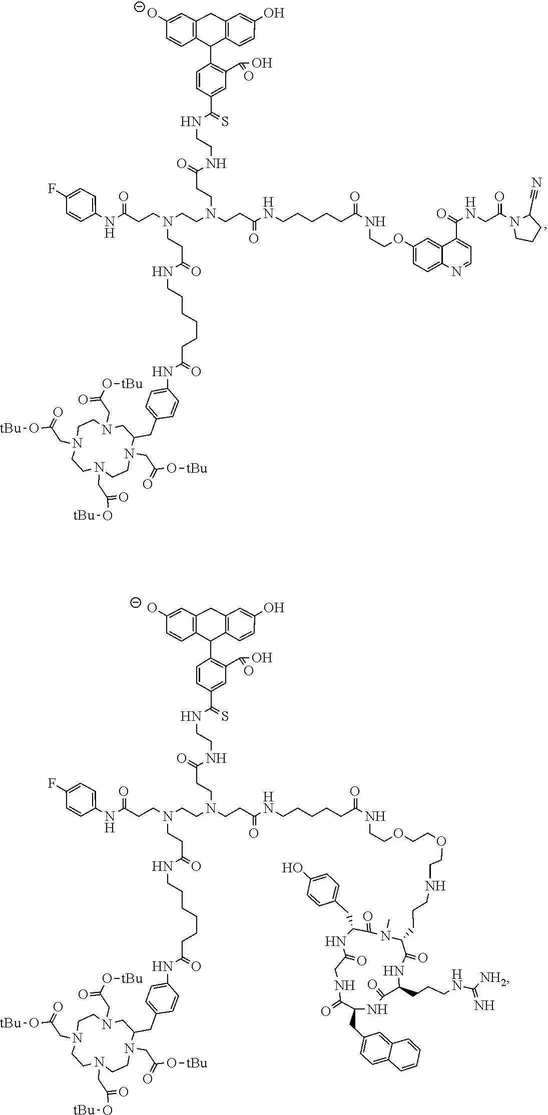

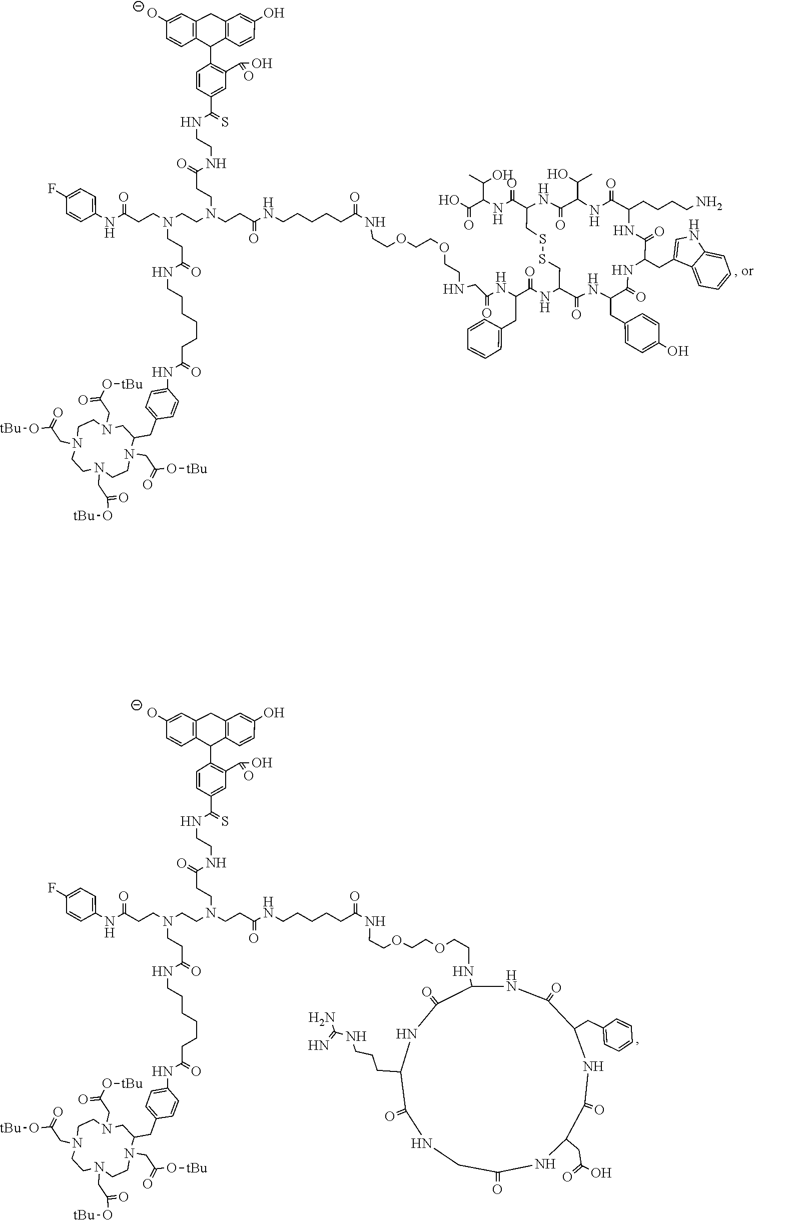

or pharmaceutically acceptable salt thereof

In embodiments, the precursor has a structure of

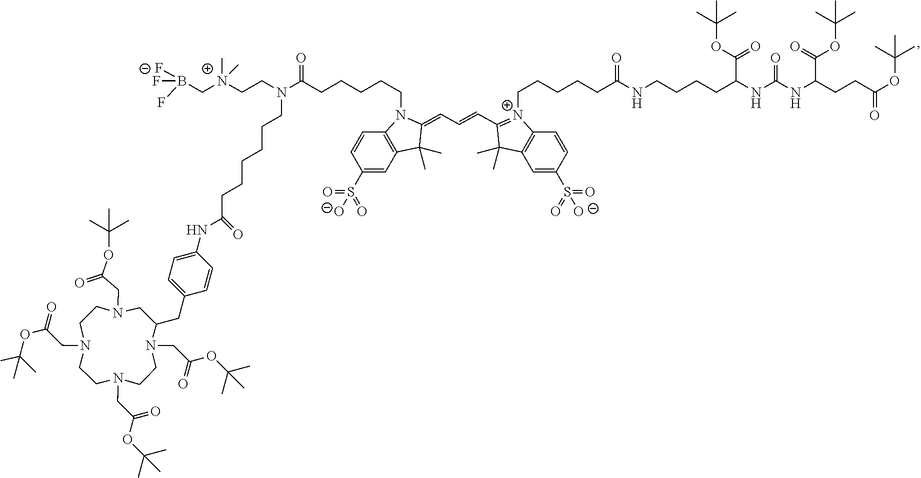

or a pharmaceutically acceptable salt thereof.
In embodiments, the precursor has a structure of

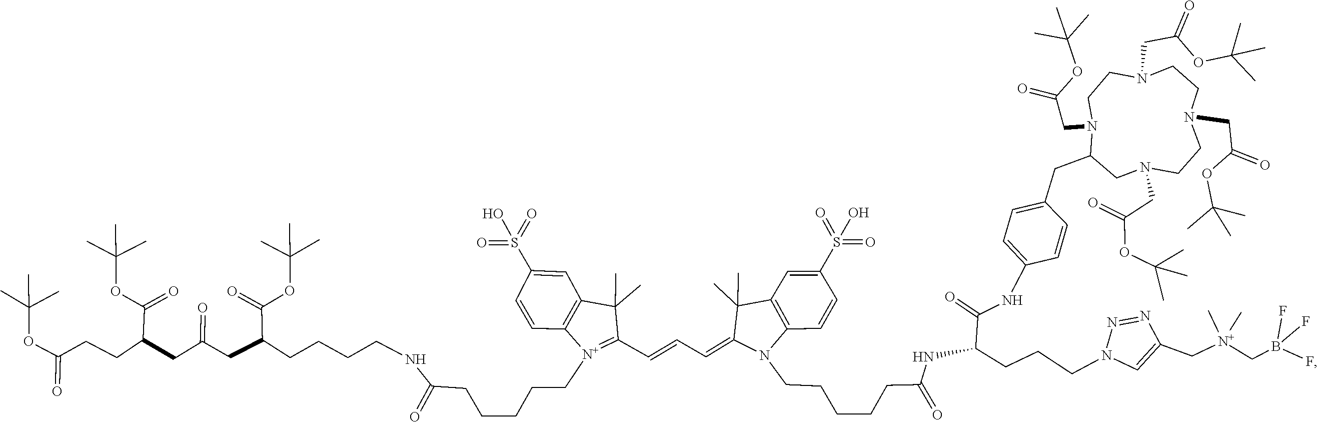

or a pharmaceutically acceptable salt thereof.
In embodiments, the precursor has a structure of

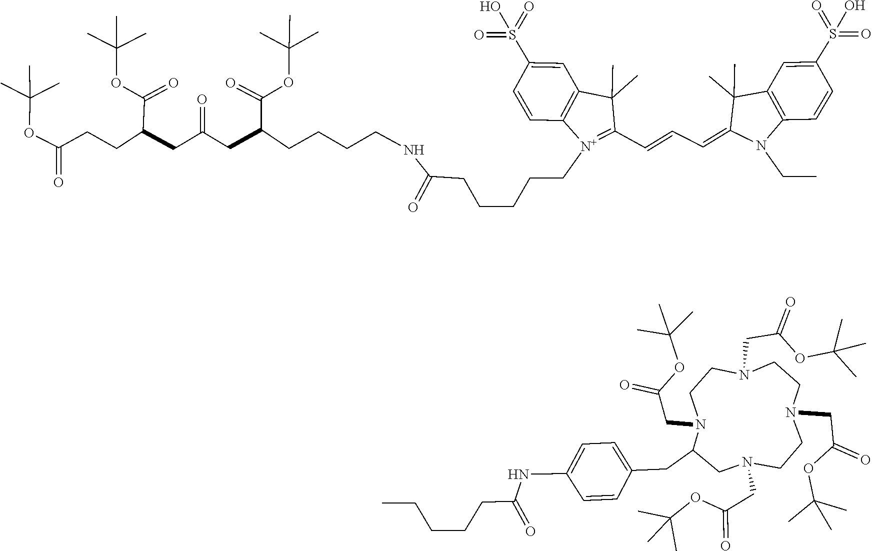

or a pharmaceutically acceptable salt thereof.

In embodiments, the compounds or the precursors thereof described herein further include a metal atom or metal ion thereof. In embodiments, the compound or precursor thereof as described herein is combined with the metal atom or the ion thereof such that the metal atom/ion may bind to a chelating ligand moiety (CL) of the compound.

### Pharmaceutical Compositions

In another aspect, provided is a pharmaceutical composition (“composition”) containing a compound of Formula (I), (I-a), (I-a-1), (II), (III), (IV-a), (IV-b), (V-a), (V-b), (VI-a), (VI-b), (X), (X-a), (XI), (XII), (XIII-a), (XIII-b), (XIV-a), or (XIV-b), or a subembodiment described herein, and one or more excipients or carriers, preferably pharmaceutically acceptable excipients or carriers.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Excipients for preparing a pharmaceutical composition are generally those that are known to be safe and non-toxic when administered to a human or animal body. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, and suitable mixtures of any of the foregoing. The particular excipients utilized in a composition will depend upon various factors, including chemical stability and solubility of the compound being formulated and the intended route of administration.

A pharmaceutical composition can be provided in bulk or unit dosage form. It is especially advantageous to formulate pharmaceutical compositions in unit dosage form for ease of administration and uniformity of dosage. The term “unit dosage form” refers to physically discrete units suited as unitary dosages for the subject to be treated; each unit containing a predetermined quantity of an active compound calculated to produce the desired therapeutic effect in association with the required pharmaceutical carrier. A unit dosage form can be an ampoule, a vial, a suppository, a dragee, a tablet, a capsule, an IV bag, or a single pump on an aerosol inhaler.

In therapeutic applications, dose may vary depending on the chemical and physical properties of the active compound as well as clinical characteristics of the subject, including e.g., age, weight, and co-morbidities. Generally, the dose should be a therapeutically effective amount. An effective amount of a pharmaceutical composition is that which provides an objectively identifiable improvement as noted by the clinician or other qualified observer. For example, alleviating a symptom of a disorder, disease or condition.

A pharmaceutical compositions may take any suitable form (e.g. liquids, aerosols, solutions, inhalants, mists, sprays; or solids, powders, ointments, pastes, creams, lotions, gels, patches and the like) for administration by any desired route (e.g. pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). In embodiments, the pharmaceutical composition is in the form of an orally acceptable dosage form including, but not limited to, capsules, tablets, buccal forms, troches, lozenges, and oral liquids in the form of emulsions, aqueous suspensions, dispersions or solutions. Capsules may contain excipients such as inert fillers and/or diluents including starches (e.g., corn, potato or tapioca starch), sugars, artificial sweetening agents, powdered celluloses, such as crystalline and microcrystalline celluloses, flours, gelatins, gums, etc. In the case of tablets for oral use, carriers which are commonly used include lactose and corn starch. Lubricating agents, such as magnesium stearate, can also be added.

In embodiments, the pharmaceutical composition is in the form of a tablet. The tablet can comprise a unit dose of a compound described here together with an inert diluent or carrier such as a sugar or sugar alcohol, for example lactose, sucrose, sorbitol or mannitol. The tablet can further comprise a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. The tablet can further comprise binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. butylated hydroxytoluene), buffering agents (e.g. phosphate or citrate buffers), and effervescent agents such as citrate/bicarbonate mixtures. The tablet may be a coated tablet. The coating can be a protective film coating (e.g. a wax or varnish) or a coating designed to control the release of the active compound, for example a delayed release (release of the active after a predetermined lag time following ingestion) or release at a particular location in the gastrointestinal tract. The latter can be achieved, for example, using enteric film coatings such as those sold under the brand name Eudragit®.

Tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecyl sulfate, magnesium aluminum silicate, and triethanolamine.

In embodiments, the pharmaceutical composition is in the form of a hard or soft gelatin capsule. In accordance with this formulation, the compound of the present disclosure may be in a solid, semi-solid, or liquid form.

In embodiments, the pharmaceutical composition is in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

In embodiments, the pharmaceutical composition is in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions can be prepared in water with the aid of co-solvent or a surfactant. Examples of suitable surfactants include polyethylene glycol (PEG)-fatty acids and PEG-fatty acid mono and diesters, PEG glycerol esters, alcohol-oil transesterification products, polyglyceryl fatty acids, propylene glycol fatty acid esters, sterol and sterol derivatives, polyethylene glycol sorbitan fatty acid esters, polyethylene glycol alkyl ethers, sugar and its derivatives, polyethylene glycol alkyl phenols, polyoxyethylene-polyoxypropylene (POE-POP) block copolymers, sorbitan fatty acid esters, ionic surfactants, fat-soluble vitamins and their salts, water-soluble vitamins and their amphiphilic derivatives, amino acids and their salts, and organic acids and their esters and anhydrides. Dispersions can also be prepared, for example, in glycerol, liquid polyethylene glycols and mixtures of the same in oils.

### Method of Use

In an aspect, provided is a method of imaging biological tissue in a subject, the method comprising administering to the subject a compound of Formula (I), (I-a), (I-a-1), (II), (III), (IV-a), (IV-b), (V-a), (V-b), (VI-a), (VI-b), (X), (X-a), (XI), (XII), (XIII-a), (XIII-b), (XIV-a), or (XIV-b), or pharmaceutically acceptable salt thereof, or a subembodiment described herein, or a pharmaceutical composition comprising same. In embodiments, the method further comprises performing one or more imaging techniques selected from computed tomography (CT), positron emission tomography (PET), single photon emission computer tomography (SPECT), magnetic resonance imaging (MRI), contrast aided (e.g., gadolinium contrast) magnetic resonance imaging (cMRI), magnetic resonance angiography (MRA), and optical or fluorescence-based imaging (FL), and combinations of any of the foregoing. In embodiments, the method comprises simultaneously performing at least two imaging techniques selected from two of the foregoing. In embodiments, the at least two imaging techniques are selected from an optical fluorescence-based imaging (FL) technique and an imaging technique selected from CT, PET, SPECT, MRI, cMRI, and MRA, or a combination thereof.

In an aspect, provided is a method of treating cancer in a subject using radioisotope therapy, the method comprising administering to the subject a compound of Formula (I), (I-a), (I-a-1), (II), (III), (IV-a), (IV-b), (V-a), (V-b), (VI-a), (VI-b), (X), (X-a), (XI), (XII), (XIII-a), (XIII-b), (XIV-a), or (XIV-b), or pharmaceutically acceptable salt thereof, or a subembodiment described herein, or a pharmaceutical composition comprising same, wherein the compound comprises a radioisotope suitable for radioisotope therapy, for example a radionuclide complexed with the CL moiety of the compound, or a radionuclide forming part of the BT moiety, such as a radiolabelled antibody suitable for radioimmunotherapy. Radionuclides, particularly beta (β)- and alpha (α) emitters) and radiohalogens and radiometals suitable for incorporation into a compound described herein are described in Table 2.

TABLE 2
Representative Therapeutic/Imaging Radionuclides

| | |
|---|---|
| β-particle emitters | ⁹⁰Y, ¹³¹I, ¹⁷⁷Lu, ¹⁵³Sm, ¹⁸⁶Re, ¹⁸⁸Re, ⁶⁷Cu, ²¹²Pb, ¹⁶⁶Ho, ⁴⁷Sc |
| α-particle emitters | ²²⁵Ac, ²¹³Bi, ²¹²Bi, ²¹¹At, ²¹²Pb, ²²⁷Th, ²²³Ra |
| Auger electron emitters | ¹²⁵I, ¹²³I, ⁶⁷Ga, ¹¹¹In, ⁷⁷Br, ^{80m}Br |

For example, radiohalogens ¹²⁵I, ¹²³I, ¹³¹I, ²¹¹At, ⁷⁷Br, and ⁸⁰Br, may be introduced via the BT moiety such as PSMA-targeting agent. These radiohalogens may be covalently bound to the targeting moiety and unlike large chelated radiometals are small enough that the entire radiolabeled PSMA inhibitor can fit within the PSMA binding cavity thereby retaining the high binding affinity. In other example, same radiolabeled prosthetic groups may be conjugated to linker-inhibitor urea conjugates to move the radiolabeled portion of the inhibitor to the exterior of the protein.

In an aspect, provided is a method of treating a disease (e.g., cancer) or a condition in a subject by administering to a subject any of the above described compounds or compositions wherein the compound comprises a therapeutic moiety, such as an RIT agent or an agent suitable for radioimmunotherapy, such that the imaging and therapy are accomplished using the same molecule. In accordance with such methods, imaging of the biological tissue may be performed prior-, in-situ, and post-treatment (e.g., surgical treatment) or therapy (e.g., radiotherapy), or any combination thereof.

In embodiments, the disclosure provides methods for image-guided surgery using a compound described herein. In embodiments, the surgery is tumor resection surgery and the BT moiety is a biomarker that targets the compound to the cancer tissue to be excised by the surgeon.

As used herein, the term “cancer” refers to all types of cancer, neoplasm, solid tumors, or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

As used herein, the term “administer” refers to any means to deliver the agent to a subject's body via any known method. In embodiments, methods of administration include, without limitation, intravenous, oral, intramuscular, subcutaneous, and intra-tumoral administration. In embodiments, administration is intravenous or intra-tumoral.

In an aspect, provided is a method of imaging biological tissue using two or more imaging techniques selected from (i) positron emission tomography (PET) or single photon emission computer tomography (SPECT); (ii) computed tomography (CT), magnetic resonance imaging (MRI), and/or magnetic resonance angiography (MRA); and (iii) optical fluorescence-based imaging, the method comprising administering to a subject a compound of Formula (I), (I-a), (I-a-1), (II), (III), (IV-a), (IV-b), (V-a), (V-b), (VI-a), (VI-b), (X), (X-a), (XI), (XII), (XIII-a), (XIII-b), (XIV-a), or (XIV-b), or pharmaceutically acceptable salt thereof, or a subembodiment described herein, or a pharmaceutical composition comprising same, wherein the compound comprises each of (1) an FCM or a CL, (2) an OP, and (3) a BT; or optionally wherein the compound comprises each of (1) an FCM, (2) a CL, (3) an OP, and (4) a BT.

In accordance with any of the embodiments described herein that comprise imaging, signals or scanned images can be acquired in-situ or ex-situ during the course of treating or diagnosing a subject according to methods known in the art, e.g., methods for conducting PET, CT, MRI, cMRI, and SPECT imaging. For example, methods for imaging biological tissue with PET or SPECT are described in P. Zanzonico, Seminars in Nuclear Medicine, vol. XXXIV, No. 2, pp. 87-111, April 2004; G. Mariani et al., Eur. J. Vucl. Med. Mol. Imaging, DOI 10.1007/s00259-010-1390-8, February 2010; and A. Rahmim et al., Nucl. Med. Commun., 29:193-207, 2008, the contents of which are herein incorporated by reference in their entirety.

In embodiments of the methods described here, a combination of two or more imaging methods advantageously enables the overlay of different types of data for improved imaging and therapy. For example, the data obtained from a high-resolution image (e.g., from MRI and/or CT) and/or a three dimensional image (e.g., from PET, MRI, cMRI and/or SPECT) can be overlayed with an optical fluorescence-based image. The optical fluorescence-based image may be obtained using an OP moiety comprising any dye moiety enabling fluorescence detection (e.g., in a range of from 400 to 1000 nm, including the visible spectrum and in the near infrared (NIR) spectrum). In a specific example, the method may comprise (1) an initial imaging step to detect a radioactive signal from the FCM moiety, e.g., using PET imaging, to determine the precise localization of the tissue or organ of interest; and (2) a second imaging step to detect the fluorescence signal emitted from the OP moiety that can be used to guide the surgeon during surgery. Accordingly, in embodiments, a compound described herein comprises at least two different detectable signals (i) radioactivity from the FCM moiety and/or the CL moiety; and (ii) fluorescence from the OP moiety. As used herein, the term ‘detectable’ refers to detectable using two or more of the imaging methods described herein.

In embodiments, provided is a method of imaging biological tissue comprising (i) administering to a subject a compound of Formula (I), (I-a), (I-a-1), (II), (III), (IV-a), (IV-b), (V-a), (V-b), (X), (X-a), (XI), (XII), (XIII-a), (XIII-b), (XIV-a), or (XIV-b), or pharmaceutically acceptable salt thereof, or a subembodiment described herein, or a pharmaceutical composition comprising same, (ii) performing an imaging technique selected from PET and SPECT, (iii) performing an imaging technique selected from CT, MRI, cMRI, and MRA; and optionally (iv) performing an imaging technique selected from an optical or fluorescence-based imaging (FL) technique, wherein the compound comprises three or more of (1) an FCM, (2) a CL, (3) an OP, and (4) a BT, and wherein the compound emits detectable signals suitable for performing each of the imaging techniques. In embodiments, the imaging techniques are each performed simultaneously or sequentially, or a combination thereof.

In embodiments, provided are methods for treating a patient suffering from internal bleeding, either caused by a tumor or other trauma, in which the BT moiety is a red blood cell or platelet and the compound localizes to the bleed site.

In embodiments, the BT moiety of the compound of Formula (I), (I-a), (I-a-1), (II), (III), (IV-a), (IV-b), (V-a), (V-b), (X), (X-a), (XI), (XII), (XIII-a), (XIII-b), (XIV-a), or (XIV-b), is a blood cell, preferably a red blood cell or platelet, and the compound comprises at least an FCM moiety, preferably comprising [¹⁸F], and optionally one or more of a CL and an OP. In embodiments, the compound comprises an FCM moiety comprising [¹⁸F] and a BT moiety comprising a blood cell, preferably a red blood cell or platelet, and the compound optionally further comprises an OP and/or CL. Such a compound may be designated [¹⁸]-RBC. This embodiment provides certain advantages, including 1) fluoridation on and at a non-carbon bearing molecule that can be used to stably radiolabel a cell and show imaging of cells by PET/SPECT, CT, and/or MRI/MRA, in vivo, 2) the ability to image radiolabeled cells by fluorescence, which can be used to confirm that the radiolabel does not transfer between cells and to image bleeding by fluorescence, and 3) use in an emergency bleeding situation. The compounds described herein having a blood cell as the BT moiety are superior to counterpart RBC imaging agents (e.g., pre-clinical chromium and gadolinium RBCs (acontrast), and current, clinical SPECT agents [^{99m}Tc]-RBC and [^{99m}Tc]-leukocyte (exametazine)) because of the higher resolution, lower quantity, and lower activities at which [¹⁸F]-RBCS can be imaged. In addition, the superior imaging potential of [¹⁸F]-RBCs can be used to image lesions that are only 1 to 4 mm in diameter in murine brains that are 10 mm in diameter. This non-invasive imaging method advantageously permits substantially higher resolution imaging than currently available. This improved imaging can be used to image small hemorrhages with higher resolution than current state of the art methods.

In embodiments, provided are methods for imaging blood flow in vivo, the methods comprising administering to a subject a compound of Formula (I), (I-a), (I-a-1), (II), (III), (IV-a), (IV-b), (V-a), (V-b), (X), (X-a), (XI), (XII), (XIII-a), (XIII-b), (XIV-a), or (XIV-b), or pharmaceutically acceptable salt thereof, or a subembodiment described herein, or a pharmaceutical composition comprising same, wherein the BT moiety comprises a cell, preferably a red blood cell or platelet. The imaging can be used, for example, to assess or monitor the progression of a hemorrhage. The hemorrhage may be located in any part of the body, including the brain (e.g., intracerebral hemorrhage, or hemorrhagic or ischemic stroke). Such assessment and monitoring may be especially important for patients with challenged renal function, where MRA and CTA are contraindicated. In embodiments, the methods further comprise simultaneous PET and CT imaging on a PET/CT instrument. In embodiments, the methods further comprise simultaneous PET/MRI imaging, for example where the superior images of brain tissue provided by MRI are desirable such in the context of a cerebral hemorrhage. In embodiments, the method is performed intraoperatively and PET imaging is used to guide a surgeon to a fluorescent probe. This is especially useful in neurosurgery and otolaryngology, using an endoscopic camera adapted to detect fluorescence. In embodiments, the methods disclosed herein may be applied to the imaging of traumatic brain injury, intestinal bleeding, renal bleeding, and internal bleeding in emergency situations, wherein the term “bleeding” may be synonymous with “hemorrhaging”. The imaging method may also be used to image perfusion, including thrombosis, such as red blood cell perfusion in vascularized composite allotransplantation (VCA). Notably, changes in blood flow are the earliest indicators of VCA complication. As the imaging method can detect changes in blood flow, the imaging method can detect complications in VCA and other transplants. The imaging method may also be used predict vascular thrombosis and indicate regions of necrosis.

The imaging method may also be used to assess or monitor transplant rejection or acceptance, such as for allotransplants, or more specifically, to image deep tissue kidney allotransplants.

In embodiments, the imaging method may include simultaneous imaging of internal biological tissue by fluorescence imaging, using fluorescence imaging techniques known in the art (e.g., F. Leblond et al., Journal of Photochemistry and Photobiology B: Biology, vol. 98 (1), 77-94, January 2010). In particular, the imaging method can be used to image early vascular thrombosis, such as in reconstructive microsurgery, by fluorescence, and deep tissue VCA by, e.g., PET/MRI. For example, the fluorescence mode, the imaging (¹⁸F-fluorophore-blood) composition can be used to monitor clinical graft viability and perfusion at high resolution, superficially (in free flaps) or in open surgical sites. Fluorescence imaging can indicate early rejection at the single cell level in superficial transplants (FL). Blood cells are optionally radiolabeled with fluorine-18 to generate a species that is molecularly (electronically) identical to the fluorescent probe. PET technology can be used to make VCA perfusion visible on PET/CT or PET/MRI devices in deep tissue transplants. The imaging (¹⁸F-fluorophore-blood) composition can be used to generate PET profiles of acute failure so that imminent graft failure could be predicted, and allotransplants can be preserved through prompt intervention. The fluorophore compositions described herein can thus prolong transplant lifetime and prevent tissue rejection in transplants. Moreover, patients that receive VCAs generally already have IV catheters in place (for analgesic delivery), thus making IV delivery of labeled blood cells a non-invasive technology.

In embodiments, provided is a method of an image-guided surgery, the method comprising administering to a subject a compound of Formula (I), (I-a), (I-a-1), (II), (III), (IV-a), (IV-b), (V-a), (V-b), (X), (X-a), (XI), (XII), (XIII-a), (XIII-b), (XIV-a), or (XIV-b), or pharmaceutically acceptable salt thereof, or a subembodiment described herein, or a pharmaceutical composition comprising same. In embodiments, the method further comprises (1) PET scanning for pre-surgical planning by distinguishing disseminated (oligometastatic disease) from localized cancer; (2) MRI or CT scanning for pre-surgical planning for additional/supplemental localization; and (3) FL scanning for intra-operative surgical guidance. In accordance with this method, the extent of a resection is clearly demarcated in three corroborative procedures, i.e., by the surgeon—in vivo observation of unresected margins in the open surgical site and ex vivo in FL/gamma scintillated analysis of resected tissue; and by the pathologist—ex vivo in FL frozen section intraoperative consult. This is made possible by the compounds of the present invention which provide persisting, cancer-specific contrast useful to multiple specialists including radiologists, urologists, and pathologists, and further provides for additional FL histology, and FL-assisted cell sorting of resolved cells following surgery.

Accordingly, provided is a method of cell-imaging that may provide post-surgical fluorescence activated cell sorted (FACS) isolation of cells with characteristics that are selected due to assistance from the subject agents. In embodiments, the method provides a small-molecule or peptide drug labeling in the cell by using the compound of Formula (I), (I-a), (I-a-1), (II), (III), (IV-a), (IV-b), (V-a), (V-b), (X), (X-a), (XI), (XII), (XIII-a), (XIII-b), (XIV-a), or (XIV-b), or pharmaceutically acceptable salt thereof.

In embodiments, the imaging method is used to assess or monitor the extent or progression of a cancer or pre-cancer, such as by imaging a tumor or pre-cancerous tissue. The cancerous or pre-cancerous tissue being imaged may be located in any part of the body, such as, for example, the prostate, breast, brain, lungs, stomach, intestines, colon, rectum, ovaries, cervix, pancreas, kidney, liver, skin, lymphs, bones, bladder, or uterus. The cancer can also include the presence of one or more carcinomas, sarcomas, lymphomas, blastomas, or teratomas (germ cell tumors).

Methods employing the compounds and compositions disclosed herein include, without limitation, the following:

(i) imaging a tumor via PET imaging, fluorescence imaging and/or optical imaging;

(ii) performing an (optionally ultrasound-guided) agent-targeted tissue biopsy via PET imaging, fluorescence imaging, and/or optical imaging;

(iii) performing a (optionally ultrasound-guided) surgical procedure to identifying sentinel lymph nodes, identifying specific sites of bleeding, or perform surgery on a tumor (e.g., removing a prostate tumor (e.g., a PSMA⁺ tumor), brain tumor (e.g., glioblastoma), head and neck cancer (e.g., squamous cell carcinoma of the head and neck), liver cancer (e.g., hepatocellular carcinoma), lung cancer (e.g., non-small cell lung cancer), colon cancer, colorectal cancer, breast cancer, sarcoma, or ovarian cancer), while employing PET imaging, fluorescence imaging, and/or optical imaging;

(iv) performing a pathology and/or histology analysis of a tissue sample (e.g., a sample obtained from a prostate tumor (e.g., a PSMA⁺ tumor), brain tumor (e.g., glioblastoma), head and neck cancer (e.g., squamous cell carcinoma of the head and neck), liver cancer (e.g., hepatocellular carcinoma), lung cancer (e.g., non-small cell lung cancer), colon cancer, colorectal cancer, breast cancer, sarcoma, or ovarian cancer) via PET imaging, fluorescence imaging, and/or optical imaging (which analysis can comprise, for example, a tumor margin analysis);

(v) determining the status (e.g., size, location and/or stage) of a tumor (e.g., a prostate tumor (e.g., a PSMA⁺ tumor), brain tumor (e.g., glioblastoma), head and neck cancer (e.g., squamous cell carcinoma of the head and neck), liver cancer (e.g., hepatocellular carcinoma), lung cancer (e.g., non-small cell lung cancer), colon cancer, colorectal cancer, breast cancer, sarcoma, or ovarian cancer)) via PET imaging, fluorescence imaging, and/or optical imaging;

(vi) monitoring the progress of cancer therapy (e.g., a targeted cancer therapy for a prostate tumor (e.g., a PSMA⁺ tumor), brain tumor (e.g., glioblastoma), head and neck cancer (e.g., squamous cell carcinoma of the head and neck), liver cancer (e.g., hepatocellular carcinoma), lung cancer (e.g., non-small cell lung cancer), colon cancer, colorectal cancer, breast cancer, sarcoma, or ovarian cancer) via PET imaging, fluorescence imaging, and/or optical imaging; and

vii) monitoring the progress of cancer surgery (e.g., a surgical removal of solid tumors such as a prostate cancer (e.g., a PSMA⁺ prostate tumor), brain, head and neck cancer, liver cancer, lung cancer, colon cancer, colorectal cancer, breast cancer, sarcoma, or ovarian cancer) via PET imaging, fluorescence imaging, and/or optical imaging (including pre-op monitoring, post-op monitoring, and monitoring during surgery).

In embodiments the methods comprise administering a compound of Formula (I), (I-a), (I-a-1), (II), (III), (IV-a), (IV-b), (V-a), (V-b), (X), (X-a), (XI), (XII), (XIII-a), (XIII-b), (XIV-a), or (XIV-b), or pharmaceutically acceptable salt thereof, at an activity ranging from 0.1 to 30 mCi, at a mass of 10 to 10000 umol intravenously at least 0-6 hours prior to PET scanning. The compounds are visible in a PET scanner for 0 min to 12 hours post-injection. Co-injected, residual compounds are visible for up to 2 weeks post injection by optical/fluorescent means. In embodiments, the methods further comprise fluorescence-guided surgery. A non positron emitting, ¹⁹F containing composition may be substituted if PET imaging is not desired.

In embodiments the methods comprise administering a compound of Formula (I), (I-a), (I-a-1), (II), (III), (IV-a), (IV-b), (V-a), (V-b), (X), (X-a), (XI), (XII), (XIII-a), (XIII-b), (XIV-a), or (XIV-b), or pharmaceutically acceptable salt thereof, at an activity ranging from 3 to 10 mCi, and mass that is less than 100 umol (−100 μg),via intra-tumoral injection prior-to or during a PET scan.

In an aspect, the method further comprises therapy with the prodrug. In embodiments, the BT moiety is configured to deliver the compound to a specific biological tissue or site, e.g the site of a tumor.

In embodiments, the disclosure provides methods for corroborative imaging following tumor resection surgery comprising administering to a subject, post-operatively, a compound disclosed herein and performing post-operative corroborative imaging on the subject using a technique suitable to detect the imaging moiety of the compound, e.g., optical, MRI, and/or PET. In embodiments, the methods may further comprise performing post-operative surgery to remove remaining tumor tissue where the imaging indicates a positive surgical resection margin.

In embodiments, the disclosure provides methods for image-guided surgery that further comprise use of a PET scanner inside the surgical suite, including robot-assisted surgeries performed with a PET scanner. In accordance with these embodiments, in the context of treating cancer, the optical signal-to-noise ratios are adjusted to allow for computer-assisted identification of margins to excise during surgery or of unresected/missed positive surgical margins following sugary. In further embodiments, tomographic computed PET data acquired during surgery is adapted to identify tumor tissue to excise, unresected tumor that must be excised prior to surgical conclusion, and involved lymph nodes for resection in real time. In this context, the PET and optical data is corroborative and allows for improved accuracy compared to the standard of care surgical procedure performed with non-contrast guided techniques. In embodiments, a surgical robot equipped with a camera is adapted for fluorescent data collection. In embodiments, multiple compounds targeted to different biological sites and comprising with different fluorescent agents are administered to the subject simultaneously in a method of guided robotic surgery. For example, the multiple compounds may include (i) a first compound having a tumor-targeted BT moiety and an optical probe moiety such as Cy3; (ii) a second compound having a lymph node targeted BT moiety and an optical probe moiety such as Cy5; and (iii) a third compound having a nerve-targeted BT moiety and an optical probe moiety such as Cy7. In this context, the method comprises administering each of the compounds simultaneously to the subject in order to assist the surgeon in identifying the cancer tissue to remove, the nerves to avoid, and any lymph nodes, including those which tumor cells have infiltrated

In an aspect, the disclosure provides a kit for making and/or using any of the above-described compounds. The kit may include, for example, the compound of Formula (I), (I-a), (I-a-1), (II), (III), (IV-a), (IV-b), (V-a), (V-b), (X), (X-a), (XI), (XII), (XIII-a), (XIII-b), (XIV-a), or (XIV-b), or pharmaceutically acceptable salt thereof, or its precursor.

Examples of kit-based preparations include protocols described in the following: (i) (preparation from a boronic ester)—Wang, Y., An, F., Chan, M., Friedman, B., Rodriguez, E. A., Tsien, R. Y., Aras, O., and Ting, R. (2017) “18F-positron-emitting/fluorescent labeled erythrocytes allow imaging of internal hemorrhage in a murine intracranial hemorrhage model.” J. Cerebral Blood Flow and Metabolism., 37(3), 776-786. PMID: 28054494; (ii) (preparation from a 19F-bearing molecule)—Kommidi, H., Guo, H. Nurili, F., Vedvyas, Y., Jin, M. M., McClure, T. D., Ehdaie, B., Sayman, H., Akin O., Aras, O., Ting, R. (2018) “18F-positron emitting/trimethine cyanine-fluorescent contrast for image-guided prostate cancer management.” J. Med. Chem. 61, 4256-4262; and (iii) Kommidi, H., Guo, H., Chen, N., Kim, D., He, B., Wu, A. P., Aras, O, Ting, R. (2017) “A [18g-positron-emitting, fluorescent, cerebrospinal fluid probe for imaging damage to the brain and spine.” Theranostics. 7, 2377-2391. (Cover article) PMID: 28744321, wherein the contents of references (i)-(iii) are herein incorporated by reference in their entirety.

In an exemplary embodiment, a kit includes the compound of Formula (I), (I-a), (I-a-1), (II), (III), (IV-a), (IV-b), (V-a), (V-b), (X), (X-a), (XI), (XII), (XIII-a), (XIII-b), (XIV-a), or (XIV-b), or pharmaceutically acceptable salt thereof, along with solutions for preparation, including (i) an acidic solution for radiolabeling (i.e. pH=2.0, pyridazine-HCl buffer; note this could be any acid, such as hydrochloric acid), and a solid phase extraction device suitable for adsorbing the labeled analyte, such as a silica-based octadecyl bonded phase (e.g., a C18 type cartridge such as those manufactured by Waters No. 186005125) for the user to purify their labeled agent. Additional optionally included solutions include one for purification (e.g., a 20-23 mL volume of water to flush contaminating [¹⁸F]-fluoride ion from the compound that is bound on the cartridge), a 4.0 mM HCl solution in ethanol (99%) (to elute the compound after removal of [¹⁸g-fluoride ion), and 1 mM phosphate buffered saline x PBS) to neutralize the compound. The kit also optionally contains a 0.22 μm filter for the agent to be administered (e.g., injected) to the patient. The user will have to provide their own ¹⁸F-fluoride ion from a cyclotron. All solutions are sterile. Optionally, the kit includes only the compound and a C18 cartridge (e.g., Waters No. 186005125), and users can make their choice of washing solutions.

In an exemplary embodiment, a kit includes a precursor compound of Formula (I), (I-a), (I-a-1), (II), (III), (IV-a), (IV-b), (V-a), (V-b), (X), (X-a), (XI), (XII), (XIII-a), (XIII-b), (XIV-a), or (XIV-b), or pharmaceutically acceptable salt thereof, along with solutions for preparation, including (i) an acidic solution for radiolabeling (i.e. pH=2.0, pyridazine-HCl buffer; note this could be any acid, such as hydrochloric acid), and a solid phase extraction device suitable for adsorbing the labeled analyte, such as a silica-based octadecyl bonded phase (e.g., a C18 type cartridge such as those manufactured by Waters No. 186005125) for the user to purify their labeled agent. Additional optionally included solutions include one for purification (e.g., a 20-23 mL volume of water to flush contaminating [¹⁸F]-fluoride ion from the compound that is bound on the cartridge), a 4.0 mM HCl solution in ethanol (99%) (to elute the compound after removal of [¹⁸g-fluoride ion), and 1 mM phosphate buffered saline×PBS) to neutralize the compound. The kit also optionally contains a 0.22 μm filter for the agent to be administered (e.g., injected) to the patient. The user will have to provide their own ¹⁸F-fluoride ion from a cyclotron. All solutions are sterile. Optionally, the kit includes only the compound and a C18 cartridge (e.g., Waters No. 186005125), and users can make their choice of washing solutions.

In embodiments, the kit includes a precursor having the structure:

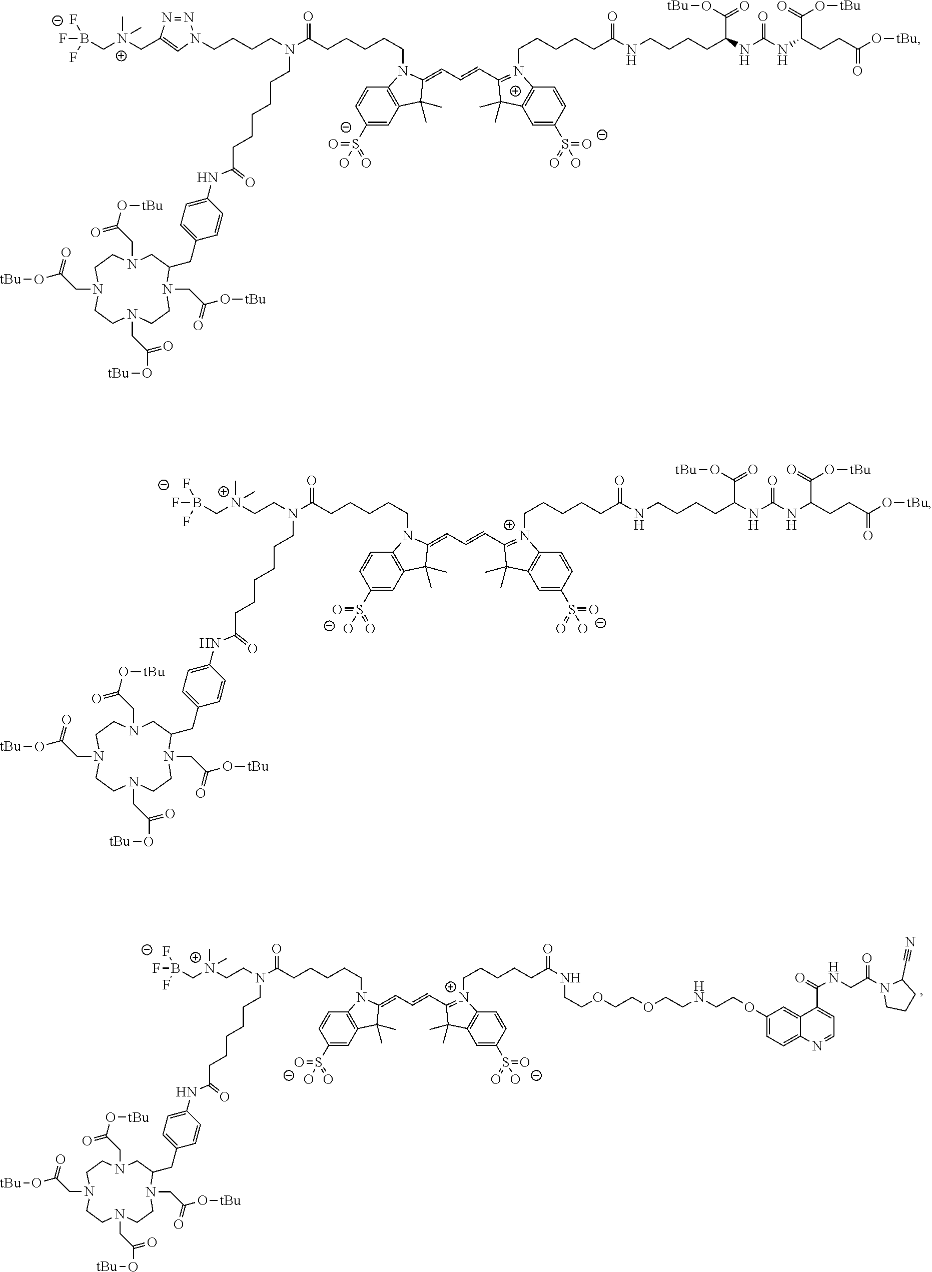

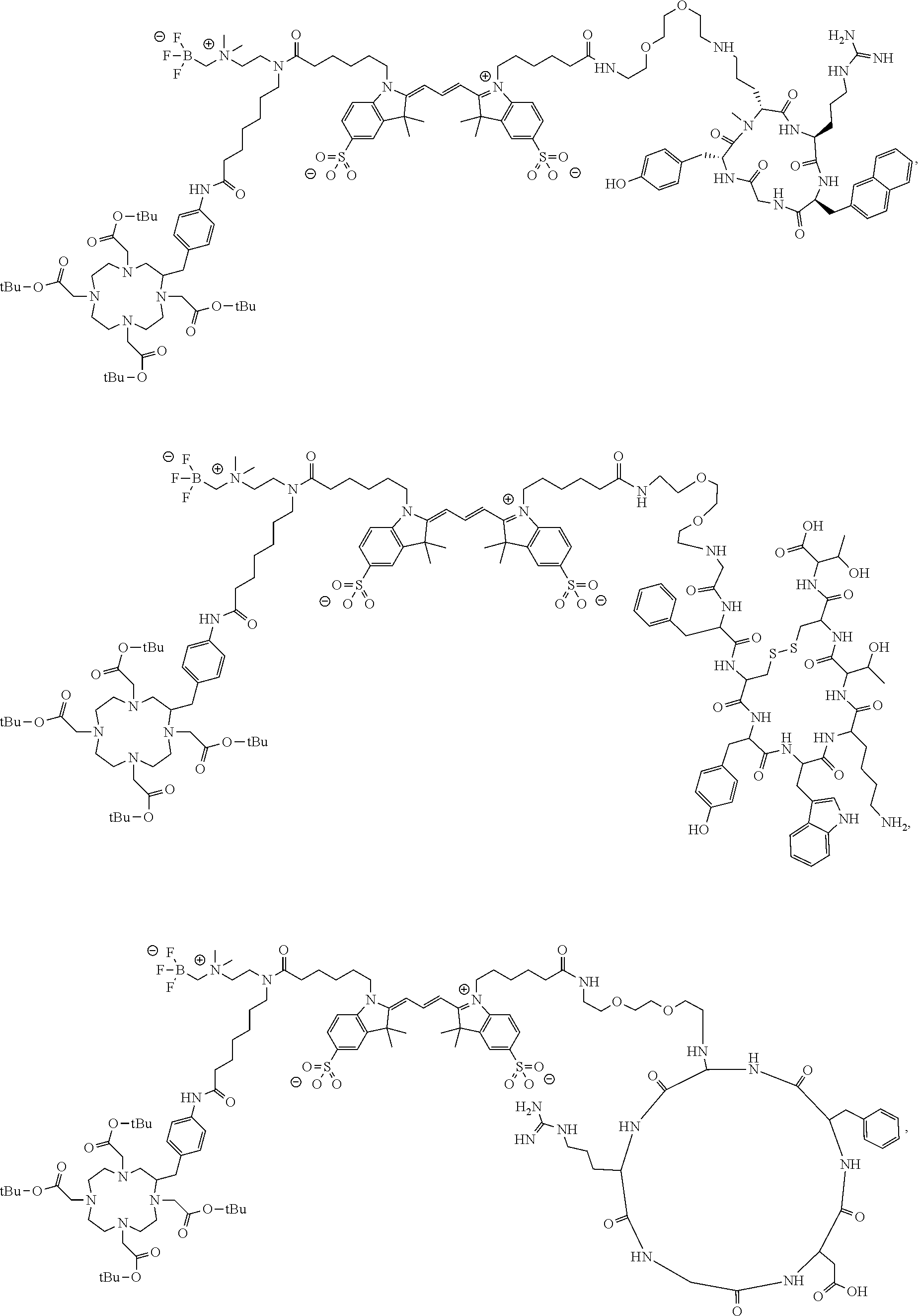

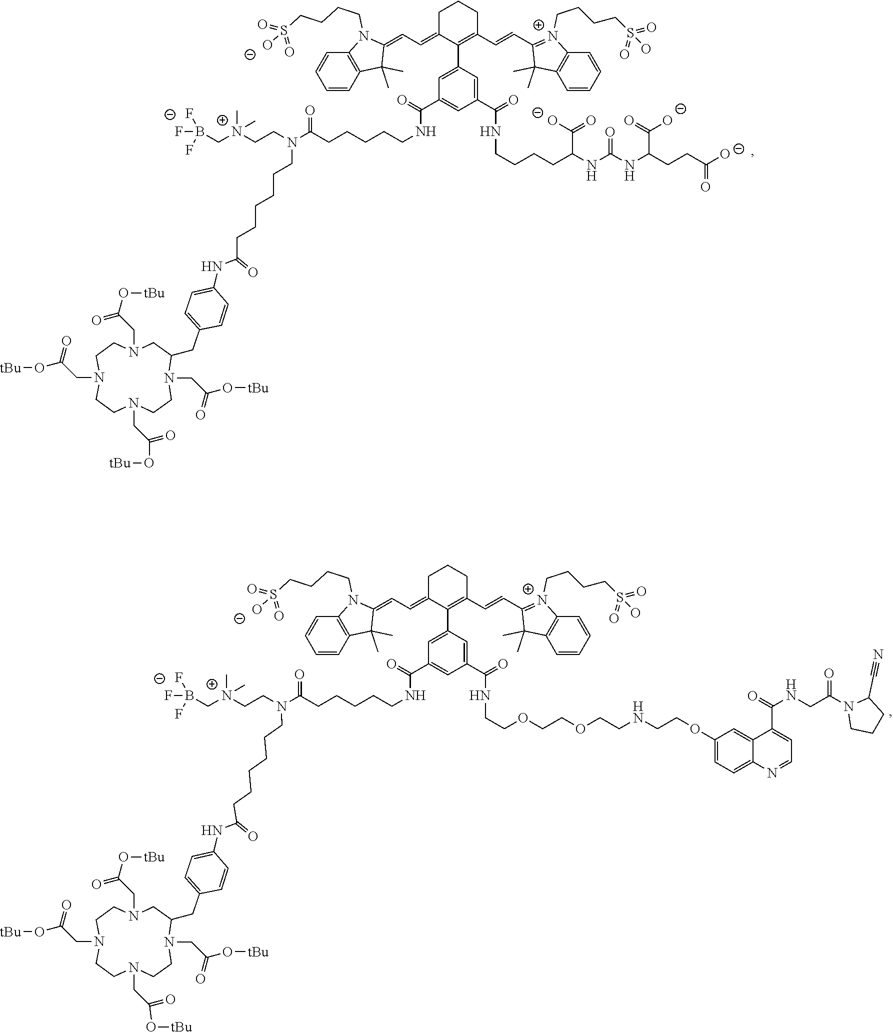

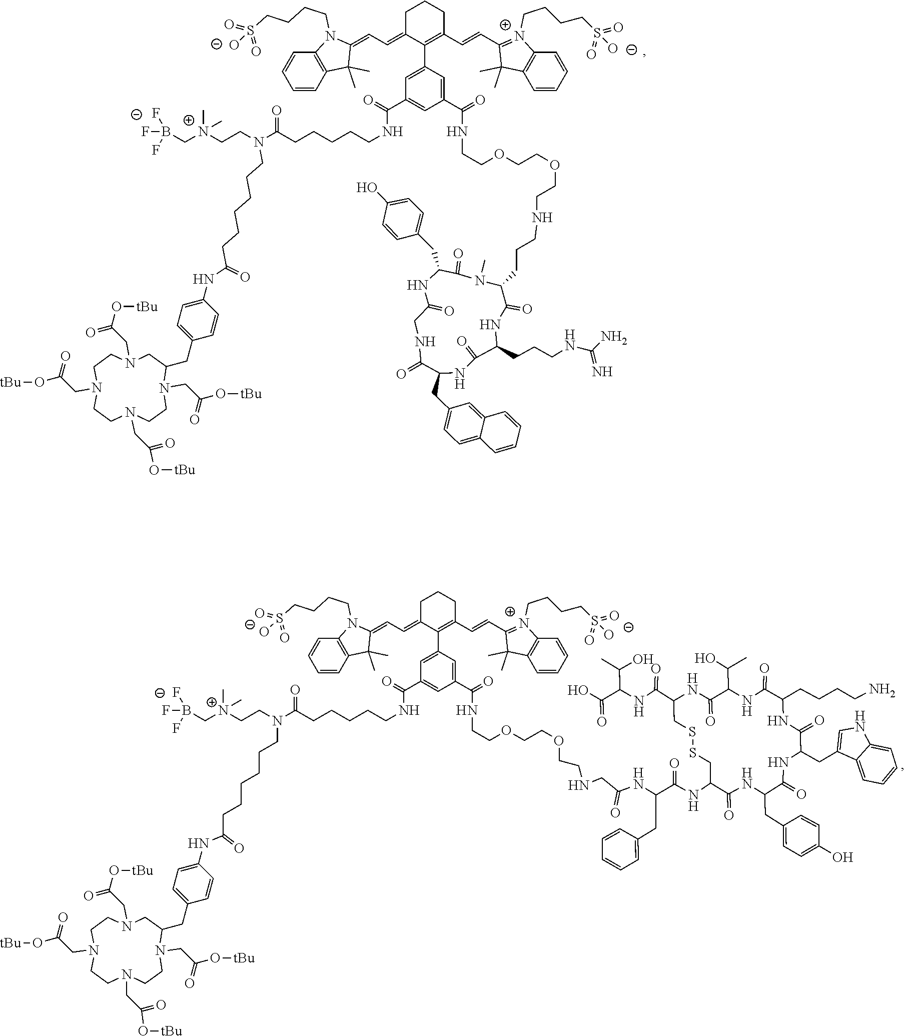

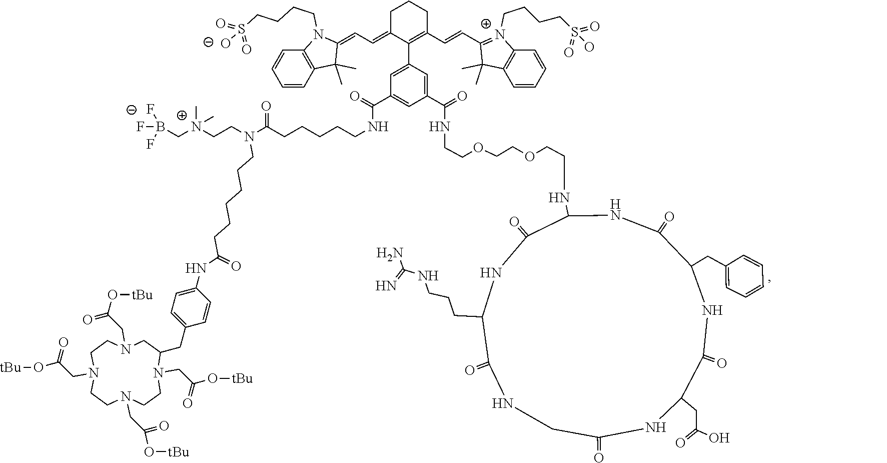

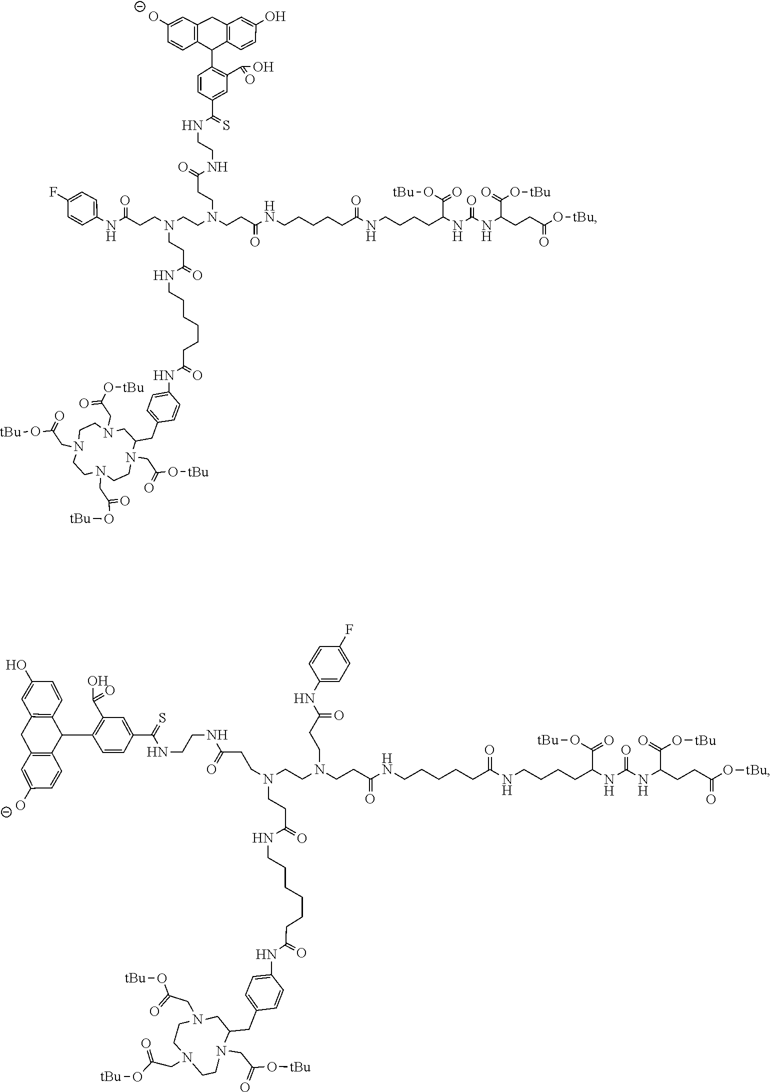

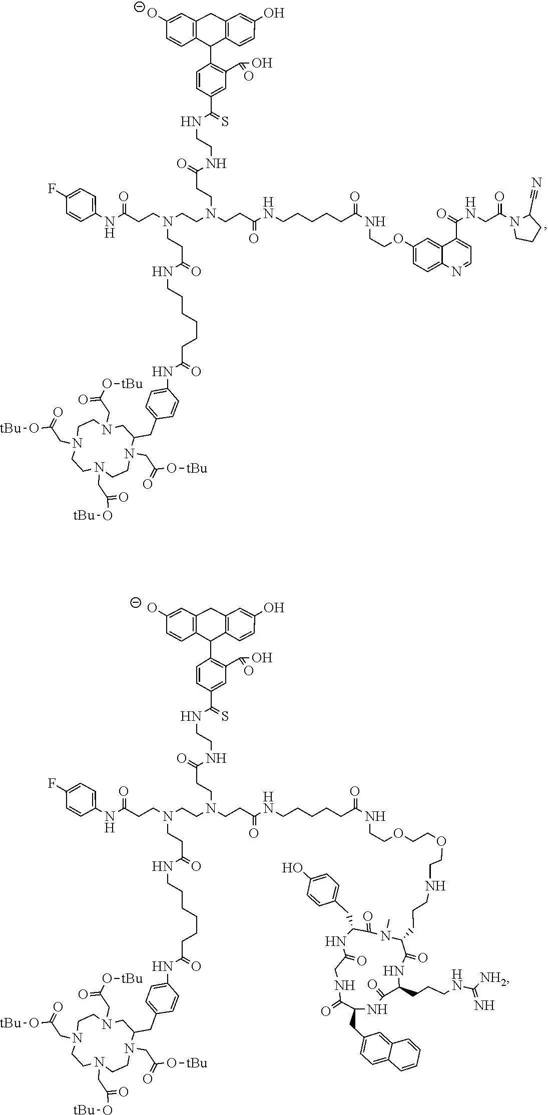

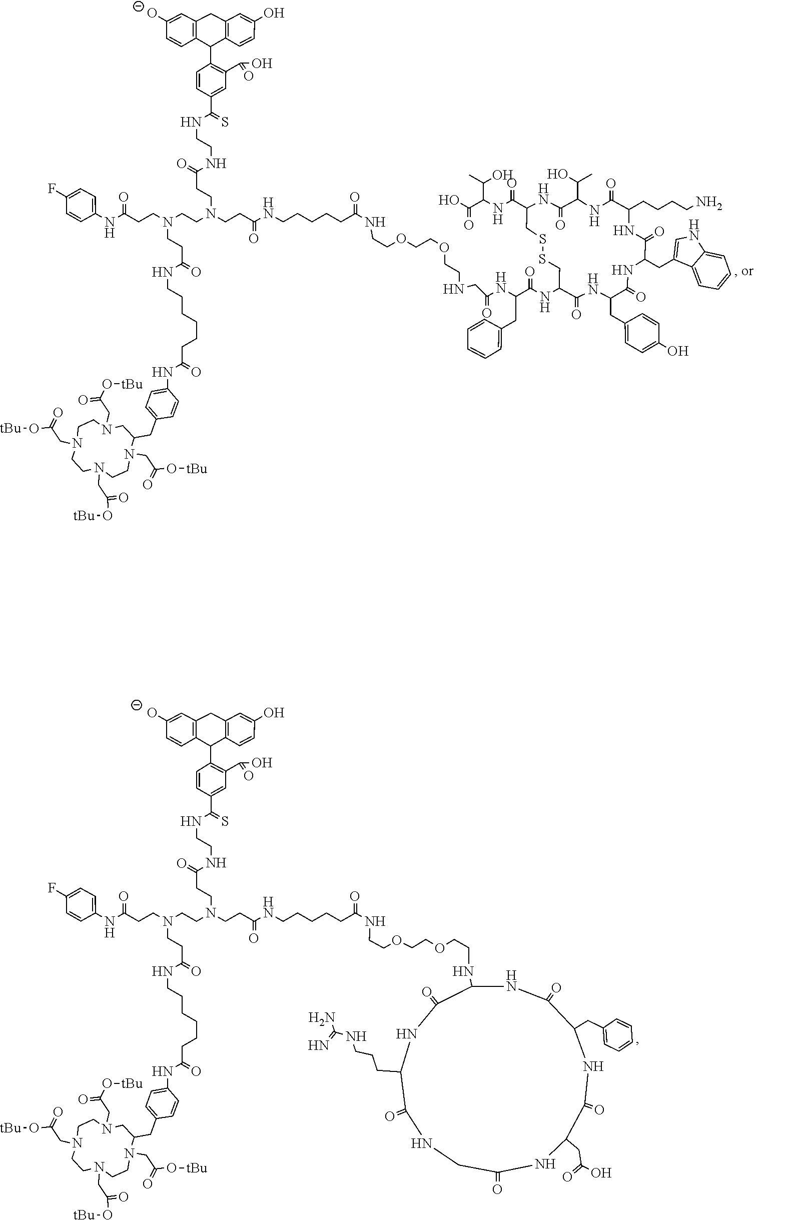

or pharmaceutically acceptable salt thereof.

In embodiments, the precursor is a precursor of Compound A-2. In embodiments, the precursor of the Compound A-2 is protected (e.g., protected with BOC). In embodiments, the precursor of Compound A-2 is

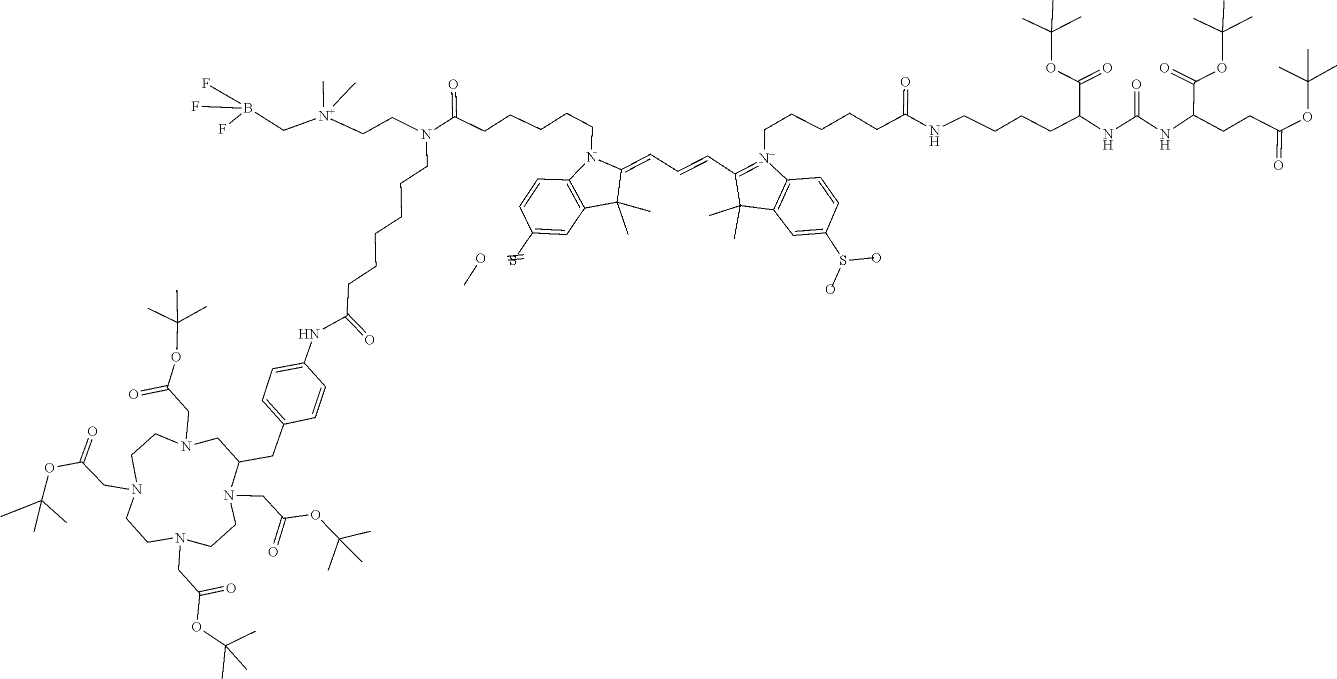

or a pharmaceutically acceptable salt thereof.

In embodiments, the kit includes the precursor of Compound A-2 above in a solid powder form and a solid phase extraction device suitable for adsorbing labeled analyte; optionally further comprising one or more sterile solutions such as purification, elution, washing, and neutralization solutions.

In embodiments, the kit includes the precursor that has a structure of

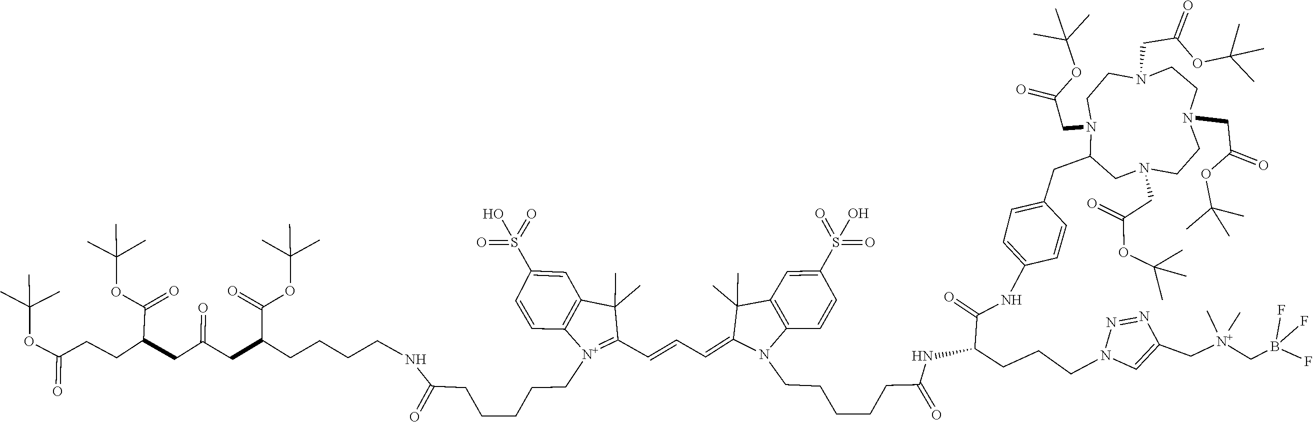

or a pharmaceutically acceptable salt thereof.

In embodiments, the kit includes the precursor that has a structure of

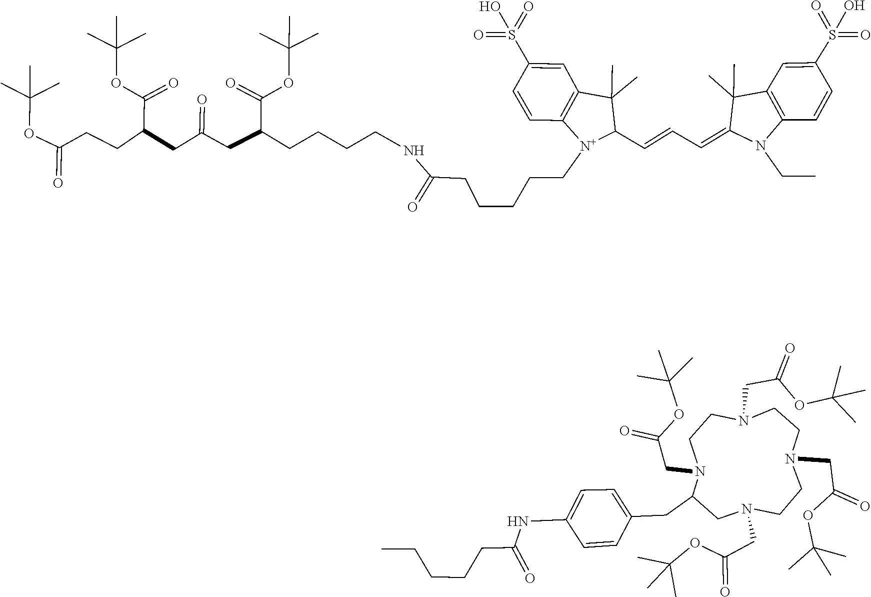

or a pharmaceutically acceptable salt thereof.

In embodiments, the kit includes a precursor described herein in a solid powder form and a solid phase extraction device suitable for adsorbing labeled analyte; optionally further comprising one or more sterile solutions such as purification, elution, washing, and neutralization solutions.

In embodiments, the kits further includes a metal atom or a metal ion thereof. In embodiments, the metal atom is a metal in Table 2. In embodiments, the metal atom is the ion form of the metal in Table 2. In embodiments, the metal atom is ⁹⁰Y, ¹³¹I, ¹⁷⁷Lu, ¹⁵³Sm, ¹⁸⁶Re, ¹⁸⁸Re, ⁶⁷Cu, ²¹²Pb, ¹⁶⁶Ho, or ⁴⁷Sc. In embodiments, the metao atom is ²²⁵Ac, ²¹³Bi, ²¹²Bi, ²¹¹At, ²¹²Pb, ²²⁷Th, or ²²³Ra. In embodiments, the metal atom is ¹²⁵I, ¹²³I, ⁶⁷Ga, ¹¹¹In, ⁷⁷Br, and ^{80m}Br. In embodiments, the metal atom is an ion of ⁹⁰Y, ¹³¹I, ¹⁷⁷Lu, ¹⁵³Sm, ¹⁸⁶Re, ¹⁸⁸Re, ⁶⁷Cu, ²¹²Pb, ¹⁶⁶Ho, or ⁴⁷Sc. In embodiments, the metal atom is an ion of ²²⁵Ac, ²¹³Bi, ²¹²Bi, ²¹¹At, ²¹²Pb, ²²⁷Th, or ²²³Ra. In embodiments, the metal atom is an ion of ¹²⁵I, ¹²³I, ⁶⁷Ga, ¹¹¹In, ⁷⁷Br, and ^{80m}Br.

In embodiments, the metal atom or the ion thereof is provided in a solution. In embodiments, the metal ion solution may be diluted with a solvent included in the kit. In embodiments, the metal atom or the ion thereof is provided in a salt. In embodiments, the metal salt may be dispersed in a solvent included in the kit.

In embodiments, a kit includes (i) dry compound (e.g., powder or crystalline), (ii) a solution of tin(IV) chloride, and (iii) HPLC grade, dry acetonitrile. The user would provide their own ¹⁸F-fluoride ion from a cyclotron. After drying this ¹⁸F-fluoride, the users would mix all reagents. In embodiments, no purification cartridge is needed (although one could use a cartridge). The user would simply precipitate out the compound with water, wash a few times with water to remove all fluoride ion, then re-suspend the compound in a PBS-buffered DMSO solution that would be passed over a 0.22 μm filter for the compound to be injected, e.g., intratumorally.

In embodiments, the kit can include one or more containers selected from the group consisting of a bottle, a vial, an ampoule, a blister pack, and a syringe. The kit can further include one or more of instructions for use, one or more syringes, one or more applicators, or a sterile solution suitable for reconstituting a compound or composition described here. For example, the kit may include instructions for mixing aliquots of these compositions, e.g., ¹⁸F-containing acidic water to give the ¹⁸F-bearing PET-visible composition. In embodiments, the kit may also include a commercial column for passing the composition through to remove contaminating fluoride ion prior to patient administration (e.g., via injection).

In an aspect, further provided is a system for an operating room (e.g., the surgical suite). In embodiments, the system may include an in-surgical suite PET scanner that can perform corroborative molecular imaging/therapy, e.g., PET/CT, PET/MRI, or PET/radioisotope. In embodiments, the system facilitates surgery performed using a PET scanner. In embodiments, the system provides confirmative PET imaging with a fluorescent endoscope or camera, e.g., on a surgical robot or on a back table histopathology cart.

The methods, kits, and systems described herein provide corroborative molecular imaging/therapy prior, during, and post-surgery, which is advantageous over conventional stand-alone PET-only, optical-only, radioisotope-only imaging agents.

### EXAMPLES

Examples have been set forth below for the purpose of illustration and to describe the best mode of the invention at the present time. However, the scope of this invention is not to be in any way limited by the examples set forth herein.

### Example 1: Synthesis of Exemplary Compound of Formula I Depicted in FIGS. 3A-3C

Reagents and Conditions:

a) 1 eq 6-Bromohexanoic Acid, p-NH₂-Bn-DOTA-tetra(t-Bu ester), HOBt (hydroxy benzotriazole), 2.5 eq. pyridine, 4.0 eq. EDC.HCl (1-ethyl-3-(3-dimethylaminopropyl)carbodiimide Hydrochloric acid salt), DMF (dimethyl formamide), RT (room temperature), N2 (nitrogen atmosphere), 6h;

b) 1.2 eq 2-(Dimethylamino)ethylamine, t-BuOK (tert-Butyl alcohol, potassium salt), MeOH (Sodium hydroxide), rt, 2h;

c) 1.0 eq. CY3.18.OH (trimethine cyanine), 1.0 eq. ACUPA ((S)-2-(3-((S)-5-Amino-1-carboxypentyl)ureido)pentanedioic Acid), 2.5 eq. HOBt, 2.5 eq. Pyridine, 4.0 eq. EDC.HCl, DMF, RT, N₂;

d) 1.0 eq. 2, 1.0 eq. 3, 2.5 eq. HOBt, 2.5 eq. Pyridine, 4.0 eq. EDC.HCl, DMF, RT, N2;

e) (1) 1.1 equiv of bromomethylboronic acid pinacol ester, DIPEA (N,N-Diisopropylethylamine), DMF/THF (2:1), rt, 1 h, (2) 3 M KHF2, 1 M HCl, 0° C. to rt, 1 h; f) 1) 0.5 mL TFA, 2h.

Metal Insertion: Metal chloride salt incubation at pH 7.5 in 100 mM Ammonium Carbonate. Radiolabeling: (if needed): 1M Pyridazine-HCl, pH=2.5, 50 mCi aqueous [18F]-fluoride ion (Specific concentration >1.5 Ci/mL), 80-90° C.

### Synthesis of tert-butyl 2,2′,2″,2′″-(2-(4-(7-bromoheptanamido)benzyl)-1,4,7,10-tetraazacyclododecane-1,4,7,10-tetrayl)tetraacetate (1)

To a solution of 6-Bromohexanoic acid (˜100 μmol) in 4 mL of dry DMF, in an oven dried 5 mL round bottom flask, p-NH₂-Bn-DOTA-tetra(t-Bu ester) (100 μmol), 1-Hydroxybenzotriazole (33 mg, 300 μmols) and 18 μL pyridine was added before condensation was started with N-3(-dimethylaminopropyl)-N′-ethyl carbodiimide hydrochloride (EDC.HCl) (25 mg, 133 μmols, Fluka 03450). The reaction was allowed to proceed for 5 hours at 27° C. under magnetic stirring under ambient atmosphere, after which a new peak corresponding to 1 was observed in UPLC/MS. The resulting solution was diluted with DMF (5 mL) and the mixture was loaded onto a preparative HPLC column. Compound 4 was isolated using a H₂O:ACN (0.05% TFA) elution gradient at a flow rate of 12 mL/min. Fractions containing 1 were lyophilized in vacuo to yield pure (OtBu)DOTA-Cy3-tert-Amine 1 as a white powder.

### Synthesis of tert-butyl 2,2′,2″2″-(2-(4-(7-(2-(dimethylamino)ethylamino)heptanamido)benzyl)-1,4,7,10-tetraazacyclododecane-1,4,7,10-tetrayl)tetraacetate (2)

To a solution of 1 (˜100 μmol) in 4 mL of methanol, 1.2 equivalents of potassium t-Butoxide is added dropwise. 1.2 eq of 2-(Dimethylamino)ethylamine is then added. The reaction is allowed to proceed for 5 hours at room temperature under magnetic stirring under ambient atmosphere, after which, a new peak corresponding to 2 will be observed by UPLC/MS. The resulting solution will be diluted with DMF (5 mL) and the mixture was loaded onto a preparative HPLC column. Compound 2 will be isolated using a H₂O:ACN (0.05% TFA) elution gradient at a flow rate of 12 mL/min. Fractions containing 2 will be lyophilized in vacuo to yield the amine 2 as a white powder.

### Synthesis of tert-butyl 2,2′,2″-(2-(4-(7-(N-(2-(dimethylamino)ethyl)amido)heptanamido)benzyl)-10-(2-isopropoxy-2-oxoethyl)-1,4,7,10-tetraazacyclododecane-1,4,7-triyl)triacetate-Cy3-(R)-di-tert-butyl 2-(3-((S)-6-acetamido-1-tert-butoxy-1-oxohexan-2-yl)ureido)pentanedioate tBu)DOTA-Cy3-tert-Amine, (4)

To a magnetically stirring solution of CY3.18.OH (25 mg, 34 μmol, synthesis adapted from Mujumdar et al., 1993) in 2 mL of dry DMF in an oven dried 5 mL round bottom flask, (S)-Di-Tert-Butyl-2-(3-((S)-6-Amino-1-(Tert-Butoxy)-1-O-xohexan-2-yl) Ureido)Pentanedioate, 1 (17 mg, 34 μmol, Astatech, CAS 1025796-31-9, Cat No. W11493), 1-Hydroxybenzotriazole (12 mg, 87 μmols) and 7 μL pyridine will be mixed before condensation is started with EDCI (42 mg, 217 μmols). The reaction will be allowed to proceed for 6 hours at 27° C., after which a mono-substituted Cy3-amide intermediate (Compound 3) will be observable by UPLC/MS. At 5 hours, compound 4 and additional EDCI (42 mg, 217 μmols, Fluka 03450) will be added to the rbf and the reaction will be allowed to proceed for 4 more hours at 28° C. The resulting solution will be diluted with DMF (4 mL) and the mixture will be loaded onto a preparative HPLC column. Compound 5 will be isolated using a H₂O:ACN (0.05% TFA), 50 min elution gradient at a flow rate of 12 mL/min. Fractions containing 5 will be lyophilized in vacuo to yield a pure pink powder.

### Synthesis of BF₃-DOTA-Cy3-PSMA (OtBu) (5)

To a room temperature, magnetically stirred solution of 4 (9.9 μmol) in dimethylformamide (DMF), 2.0 mL of neat disiopropyl ethylamine (2 μL, 9.9 μmol), followed by (Bromomethyl)boronic acid pinacol ester (2.6 mg, 12.0 μmol) was added. The reaction was allowed to proceed at RT for 2.0 hours or until complete consumption of starting material and formation of desired N-alkylated product is observed by UPLC-MS. Without further purification, the boronate was converted to a trifluoroborate. A 1 M solution of potassium hydrogen fluoride (KHF₂, 20 μL) followed by 3 M of hydrochloric acid (HCl, 10 μL) is added to the reaction pot at 0° C. The reaction is stirred for 1 hour at room temperature, under ambient atmosphere. The formation of 5 is confirmed by UPLC-MS. The resulting solution was quenched with 5 μL of 28% NH₄OH, filtered, washed with DMF (1.5 mL), and purified by preparative HPLC, using a H₂O:ACN (0.05% TFA), 50 min elution gradient at a flow rate of 12 mL/min. A linear gradient of increasing ACN from 10% to 70% between 0 to 40 min, followed by a linear gradient of increasing ACN from 70% to 90% between 40 to 50 min, was used to elute 5. The fractions containing the desired product were lyophilized in vacuo to yield pure (BOC)-protected 5 as a pink powder. Isolated yield.

### Synthesis of BF₃-DOTA-Cy3-PSMA (6)

Neat trifluoroacetic acid (TFA) (1.0 mL) was added to a stirred solution of 5 (5.0 μmol) at 0° C. Under continuous stirring, the reaction was warmed to room temperature (25° C.) over a 1-hour period. A clean conversion of the BOC (tert-butyloxycarbonyl) protected acid into a corresponding acid was observed by UPLC/MS. TFA was removed from the mixture under vacuum. The resulting solid was dissolved in DMF (1.0 mL), and purified by preparative HPLC. The fractions containing the desired product were lyophilized in vacuo to yield chemically pure 6 as a pink powder.

### Discussion

In FIGS. 3A-3C, the tert-butyloxycarbonyl protecting group or tert-butoxycarbonyl protecting group (also referred to as a “BOC” group), designated “O-tBu” in the figures, should remain on the chelator until step fin order to avoid self reaction between the amine and carboxyl groups of the chelator. Failure to incorporate acid-BOC protection may result in failure of the reaction scheme. BOC protection is preferred over other protecting groups due to the presence here of an organic fluorophore. Organic fluorophores are reasonably chemically reactive due to the presence of extended pi-conjugation. Base and platinum-hydrogen labile protecting groups will destroy organic fluorophores, either by reducing or irreversibly reacting with them. In addition, other protecting groups may interfere with the use of product 2, a reagent that exploits the reactive properties of secondary and tertiary amines.

Care should be taken to ensure that the reagents do not contain divalent or trivalent counter cations. The presence of di- or trivalent cations may cause an irreversible reaction with the chelator (chelation) and/or deprotection of the OtBu protected chelator.

Compound 2, produced after steps a and b, is an important intermediate. Compound 2 includes both an unhindered (dimethyl-substituted) tertiary amine and a secondary amine. The presence of an unhindered tertiary amine on product 4 allows our molecule to bear fluoride following site-specific halo methylboronic pinacolate reaction and subsequent fluoride treatment (step e). The secondary amine on Compound 2 allows it to undergo site-specific amide formation in step d. This strategic use of both tertiary and secondary amines on Compound 2 prevents side reactions in steps d and e. In other words, the dimethyl substituted tertiary amine will not undergo amide formation in step d, while the N-alkyl-amide formed will not undergo reaction with halo methylboronic pinacolate in step e. Additionally, tertiary amines and secondary amines will not react with, or deprotect, the chelator. Tertiary amines and secondary amines will not undergo most side reactions in step d if a biological targeting ligand or fluorophore contains a necessary reactive acid/amine/nucleophile. Compound 2 is also needed to give site specific reactions in steps d and e.

In order to increase yield, step d may be performed in a 1-pot 2-step reaction. This avoids the possibility of reduced yield from the isolation of an Acid-Cy3-ACUPA following step c. In particular, the secondary amine (i.e the product of reaction b) must be the limiting reagent. This reagent must follow reaction after ACUPA in the second step of the two-step reaction in step d as the chelator-secondary amine is costly and/or time consuming to make.

In step c, an ACUPA or substituted biological targeting ligand reagent must contain only a single reactive amine. All other necessary amines and acids on the biological entity should be chemically protected.

Ideally, reaction of the biological targeting ligand in step c should meet the following conditions: a) acid labile protecting groups are used (OtBu is preferred) because alternative base, nucleophile, strong electrophile and platinum-hydrogen deprotection strategies will destroy the organic fluorophore; b) the molecule should contain a single reactive amine to prevent over reaction (2x amide formation) of the bis-acid, cy3 (step c); and c) precise stoichiometric control must be exerted in step c to prevent over reaction of the bis-acid, cy3.

The synthesis of Compound 4 (FIG. 3C) is set up by using Compound 2. Compound 4 contains a single unprotected tertiary amine, i.e. only one reactive tertiary amine. The production of a molecule bearing this single unhindered (dimethyl-substituted) tertiary amine can be important for high yielding, site-specific trifluoroborate functionalization in step e. In addition, Compound 4 does not bear additional non-protected secondary or primary amines or acids that are unprotected and would interfere with the site specific reaction in step e.

In FIG. 3C, the secondary amine, Compound 4, may react with a halo methylboronic acid pinacol ester to give a boron pinacolate which is then treated with potassium hydrogen fluoride ion to give a (¹⁹F, or ¹⁸F)-trifluoroborate near a quaternary amine in step e. Potassium hydrogen fluoride may be used to convert the boron pinacolate to the trifluoroborate as hydrogen fluoride or other fluoro acids/electrophiles/nucleophiles may result in OtBu Deprotection/fluorophore destruction.

Compound 5 is the most preferred product for bulk long-term precursor active pharmaceutical ingredient storage, although Compound 4 can also undergo long term storage. Deprotection of Compound 5 should be performed with trifluoroacetic acid. Use of other fluoroacids may destroy the organic fluorophore.

After step f, TFA deprotection, the lifetime of Compound 6 (Compound A-2 in paragraph [120]) is very limited unless the chelator is filled with the desired metal; be it a radioactive metal (177 Lu or 225 Ac- for alpha therapy) or a non-radioactive metal (¹⁷⁵Lu or Gd—for fluorescence guided surgery or 18F PET imaging). If the chelator in Compound 6 is not filled immediately, B-F defluoridation/decomposition may occur. Compound 6, after metal addition is less-reactive and is the precursor for 19F/18F substitution for PET imaging or can be used in its non-radioactive form for fluorescence imaging.

PET radiolabeling (¹⁹F/¹⁸F) can occur either before, during or after metal chelation. However, the chelator in Compound 6 should be filled with a metal quickly to avoid defluoridation/decomposition.

As can be seen from this example, synthesizing a compound that combines a radiolabelled moiety, such as (¹⁹F/¹⁸F), with an optical imaging agent, such as an organic fluorophore, and a chelator, such as DOTA, is non-trivial. To our knowledge, no such multimodality (optical/PET/RIT) compounds have not been described or synthesized prior to the present invention. Traditional methods for 18F radiolabeling, such as fluoride-carbon substitution, would destroy all current FDA-approved fluorophores.

### Example 2: Synthesis of Compound A-23

### S-2-(4-Aminobenzyl)-1,4,7,10-tetraazacyclododecane tetra-tert-butylacetate and (5)-di-tert-butyl 2-(3-((S)-6-amino-1-(tert-butoxy)-1-oxohexan-2-yl)ureido)pentanedioate were Purchased

HPLC-MS conditions: analytical, reverse phase HPLC-MS were performed on a Agilent 1200 HPLC Prep/Analytical LCMS system. All analytical HPLC were performed using an a10-90 (Water-Acetonitrile), 20 min (or 10 min for A-23-Intl only) gradient, where both solvents contained 0.05% Trifluoroacetic acid. Analytes were run through a Phenomenex Luna, 10 um, C18(2), 100A, 250×4.6 mm column (P/No. 00G-4253-E0, SN H21-218267) at a flow rate of 1 mL/min. Analyses were performed using an Agilent G7165A Multiwavelength Detector placed upstream from an in-line Agilent G6125B Mass Spectrometer. Wavelength as indicated.

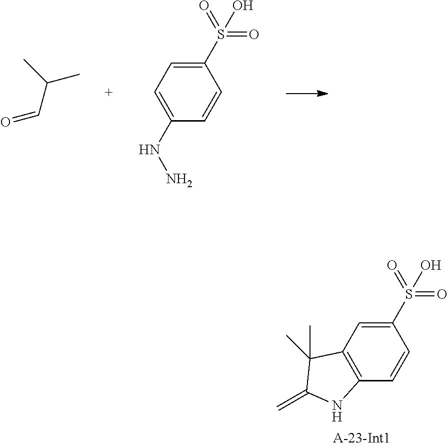

A 50 mL round bottom flask was charged with a magnetic stir bar and glacial acetic acid (6 ml, 104 mmols). 3-methyl-2-butanone (3.4 ml, 31 mmols) was added to the flask before the flask was transferred to a hotplate and magnetically stirred. To the stirring solution, 4-Hydrazinylbenzenesulfonic acid (2 g, 10.2 mmols) was added and the reaction was heated to 110° C. The reaction was allowed to proceed for 24 to 72 h at 110° C., during which a pink to deep-purple color change was observed. The resulting solution was diluted with 20 mL of water and then shell frozen at −26° C. The frozen reaction was lyophilized in vacuo to give A-23-Intl as a hygroscopic purple solid (˜90% pure by HPLC at 350 nm). HPLC-MS: 239.9 (MH+), analytical HPLC retention time=13.9 min (210 nm).

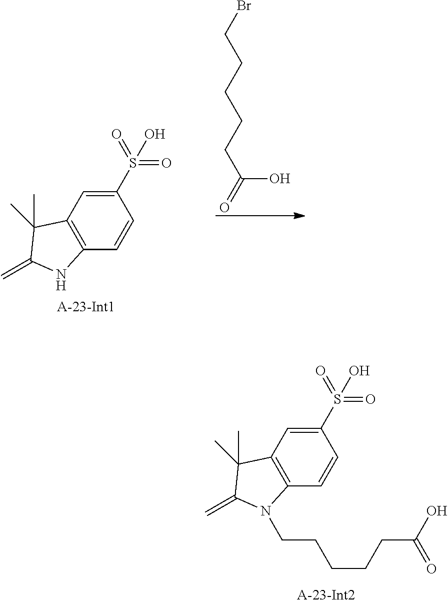 [SMILES: C=C1Nc2ccc(S(=O)(=O)O)cc2C1(C)C ; O=C(O)CCCCCBr ; C=C1N(CCCCCC(=O)O)c2ccc(S(=O)(=O)O)cc2C1(C)C]

A 50 mL round bottom flask was charged with a magnetic stir bar and A-23-Intl (2.4 g, 10.2 mmols). 1,2 dichlorobenzene (5.6 mL) and 6-bromohexanoic acid (5 mL) were added to the vial respectively. The vial was transferred to a magnetic stirrer and saturated potassium hydroxide-isopropyl alcohol solution (5 mL, 2.6 M) was added to the solution. The reaction was heated to 110° C. and proceeded for 48 h, whereupon the reaction was allowed to cool to room temperature, which resulted in a biphasic mixture consisting of a dense purple oil containing the product, and a supernatant. The supernatant was decanted and 5 ml of water was added to the oil. Titration of the oil with water resulted in a solution suitable for preparative HPLC (10 μm C18(2) 250×21.2 mm 100 A preparative column using a H₂O/ACN (0.05% TFA), 20 min, 0% to 25% elution gradient at a flow rate of 15 mL/min) to give 0.67g A-23-Int2 (>95% purity by HPLC at 250 nm). HPLC-MS: 354.1 (MH+), analytical HPLC retention time=13.1 min (210 nm).

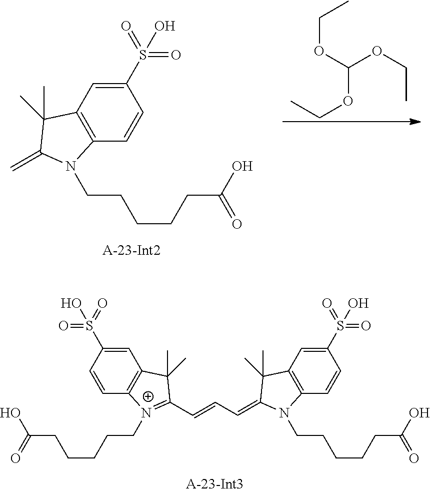

1-(5-carboxypentyl)-2-((E)-3-((E)-1-(5-carboxypentyl)-3,3-dimethyl-5-sulfoindolin-2-ylidene)prop-1-en-1-yl)-3,3-dimethyl-5-sulfo-3H-indol-1-ium salt (A-23-Int3) was synthesized from A-23-Int2 and triethoxymethane. A 5 mL vial was charged with A-23-Int2 (0.67 g, 2.8 mmols). N,N-dimethylformamide (2 mL) was added to give a fully solubilize A-23-Int2. Pyridine (1.33 mL, 16.5 mmols), and triethylorthoformate (1.33 mL of, 8 mmols) were then added to the mixture. The vail was transferred to a stir plate/heater at 110° C. The reaction was heated at 110° C. for 2h. A color change from dark purple to an intense bright pink was observed within minutes following heating. After 2 hours the reaction was separated on a 10 μm C18(2) 250×21.2 mm 100 A preparative column using a H₂O/ACN (0.05% TFA), 20 min, 10% to 90% elution gradient at a flow rate of 15 mL/min to give A-23-Int3 (0.42 g, >89% pure by HPLC at 550 nm). HPLC-MS: 717.0 (M+), analytical HPLC retention time=9.4 min (250 nm).

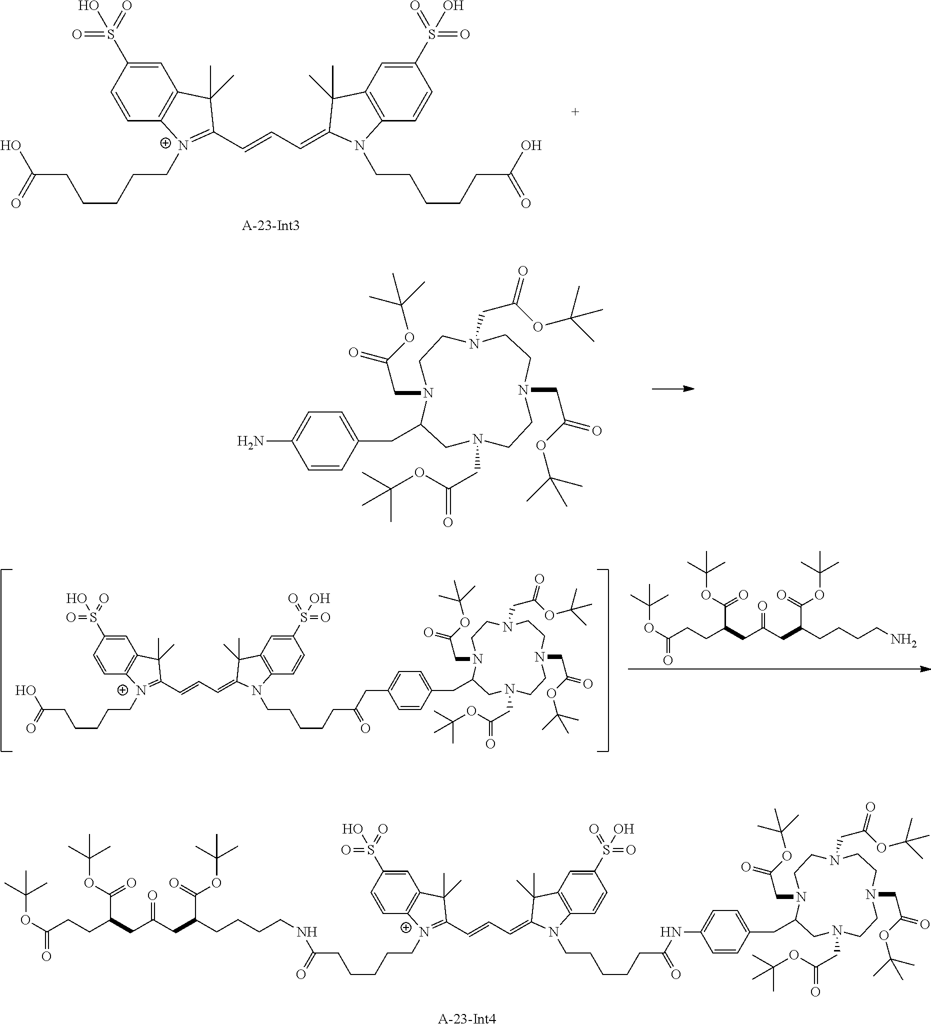

1-(6-(((5S,9R)-12-(tert-butoxy)-5,9-bis(tert-butoxycarbonyl)-7,12-dioxododecyl)amino)-6-oxohexyl)-2-((E)-3-((E)-3,3-dimethyl-1-(6-oxo-6-((4-((1,4,7,10-tetrakis(2-(tert-butoxy)-2-oxoethyl)-1,4,7,10-tetraazacyclododecan-2-yl)methyl)phenyl)amino)hexyl)-5-sulfoindolin-2-ylidene)prop-1-en-1-yl)-3,3-dimethyl-5-sulfo-3H-indol-1-ium salt (A-23-Int4) was synthesized from A-23-Int3. S-2-(4-Aminobenzyl)-1,4,7,10-tetraazacyclododecane tetra-tert-butylacetate (5 mg, 6 μmol), 140 μL of N,N-dimethylformamide, A-23-Int3 (6.2 mg, 8 μmol), 1-hydroxybenzotriazole (6 mg, 43 μmol), and pyridine (10 μL) were added before condensation was started with 1-Ethyl-3-(3-dimethylaminopropyl)carbodiimide (18 mg, 94 μmol). The condensation reaction was allowed to proceed at room temperature for ˜24 h. After 24 h (S)-di-tert-butyl 2-(3-((S)-6-amino-1-(tert-butoxy)-1-oxohexan-2-yl)ureido)pentanedioate (6 mg, 12.3 μmol) and additional 1-Ethyl-3-(3-dimethylaminopropyl)carbodiimide (18 mg, 94 μmol) were added to the reaction mixture. The reaction was left at room temperature for 72h before the reaction was diluted with N,N-dimethylformamide (300 μL) and the mixture was loaded onto a preparative HPLC. HPLC separation took place on a 10 μm C18(2) 250×21.2 mm 100 A preparative column using a H₂O/ACN (0.05% TFA), 20 min, 10% to 90% elution gradient at a flow rate of 15 mL/min. HPLC-MS: 1902.4 (M+), analytical HPLC retention time=17.5 min (250 nm).

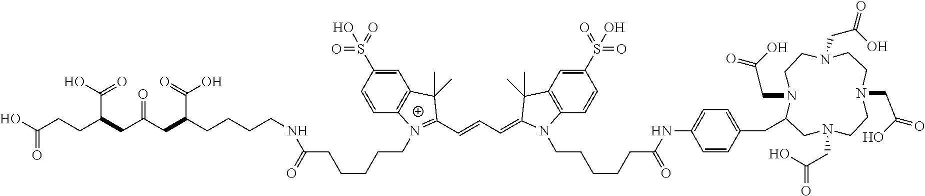

Synthesis of Compound A-23. Powdered A-23-Int4 (˜1 mg) was weighed and added to an empty 1.5 mL vial. A 200 μL volume of trifluoroacetic acid was added, and the reaction was allowed to proceed for 2 hours. After 2 hours, the reaction was diluted with 1 mL of HPLC grade water. The entire reaction was frozen and lyophilized in vacuo to give Compound A-23 trifluoroacetate as a pink solid. HPLC-MS: 1509 (M+), analytical HPLC retention time=7.84 min (550 nm).

### Example 3: Synthesis of Compound A-22-Int4

Analytical HPLC-MS conditions were as described in Example 2.

S-2-(4-Aminobenzyl)-1,4,7,10-tetraazacyclododecane tetra-tert-butylacetate and (5)-di-tert-butyl 2-(3-((S)-6-amino-1-(tert-butoxy)-1-oxohexan-2-yl)ureido)pentanedioate were purchased. N-Propargyl-N,N-dimethylammoniomethyl trifluoroborate was synthesized according to Angew. Chem. Int. Ed. 2014, 53, 11876.

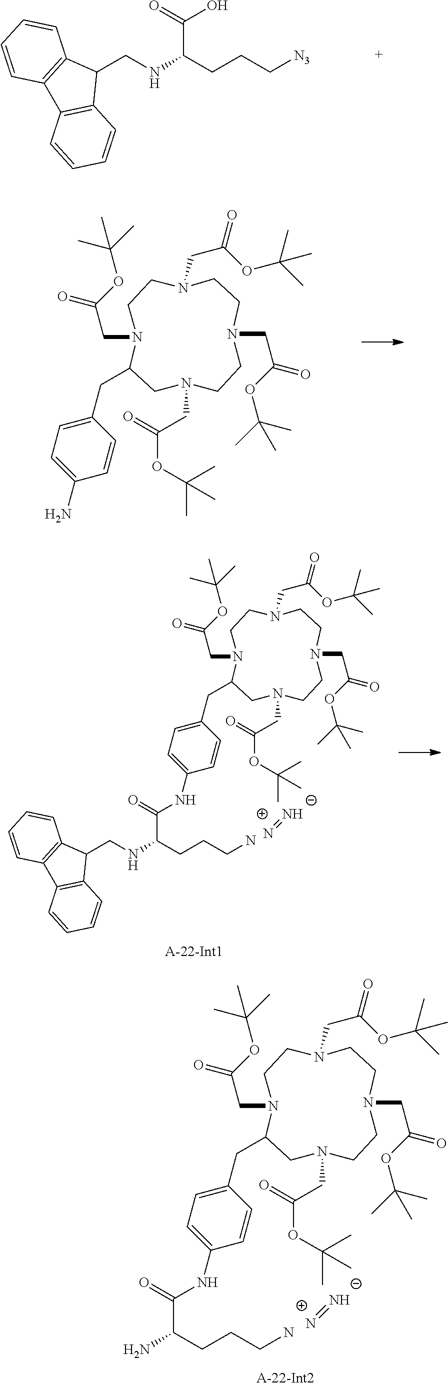

Tetra-tert-butyl 2,2′,2″,2′″-(2-(4-((S)-2-amino-5-azidopentanamido)benzyl)-1,4,7,10-tetraazacyclododecane-1,4,7,10-tetrayl)tetraacetate (A-22-Int2) was synthesized by Fmoc deprotection of tetra-tert-butyl 2,2′,2″,2″-(2-(4-((S)-2-(((9H-fluoren-9-yl)methyl)amino)-5-azidopentanamido)benzyl)-1,4,7,10-tetraazacyclododecane-1,4,7,10-tetrayl)tetraacetate (A-22-Int1), itself synthesized by amide coupling of (S)-2-(((9H-fluoren-9-yl)methyl)amino)-5-azidopentanoic acid and tetra-tert-butyl 2,2′,2″,2′″-(2-(4-aminobenzyl)-1,4,7,10-tetraazacyclododecane-1,4,7,10-tetrayl)tetraacetate. To an empty 1.5 mL vial, S-2-(4-Aminobenzyl)-1,4,7,10-tetraazacyclododecane tetra-tert-butylacetate (25.5 mg, 30 μmol), N,N-dimethylformamide (350 μL), S-5 azido-2-(Fmoc-amino)pentanoic acid (16.9 mg, 44 μmol), 1-hydroxybenzotriazole (6 mg, 157 μmol), and pyridine (37 μL) were added before condensation was started with 1-Ethyl-3-(3-dimethylaminopropyl)carbodiimide (75 mg, 390 μmol). The resulting reaction slurry became fully soluble as the reaction proceeded. The reaction was left at room temperature for 24 h before 300 μL of a 20% piperidine, N,N-dimethylformamide fluorenylmethyloxycarbonyl (Fmoc) deprotecting solution was added. Fmoc deprotection was allowed to proceed for 3 hours at room temperature before the reaction was diluted with N,N-dimethylformamide (300 μL), and the mixture was loaded onto a preparative HPLC. HPLC separation took place on a 10 μm C18(2) 250×21.2 mm 100A preparative column using a H2O/ACN (0.05% TFA), 20 min, 10% to 90% elution gradient at a flow rate of 15 mL/min. HPLC-MS: 873.4 (M+), analytical HPLC retention time=9.7 min (250 nm).

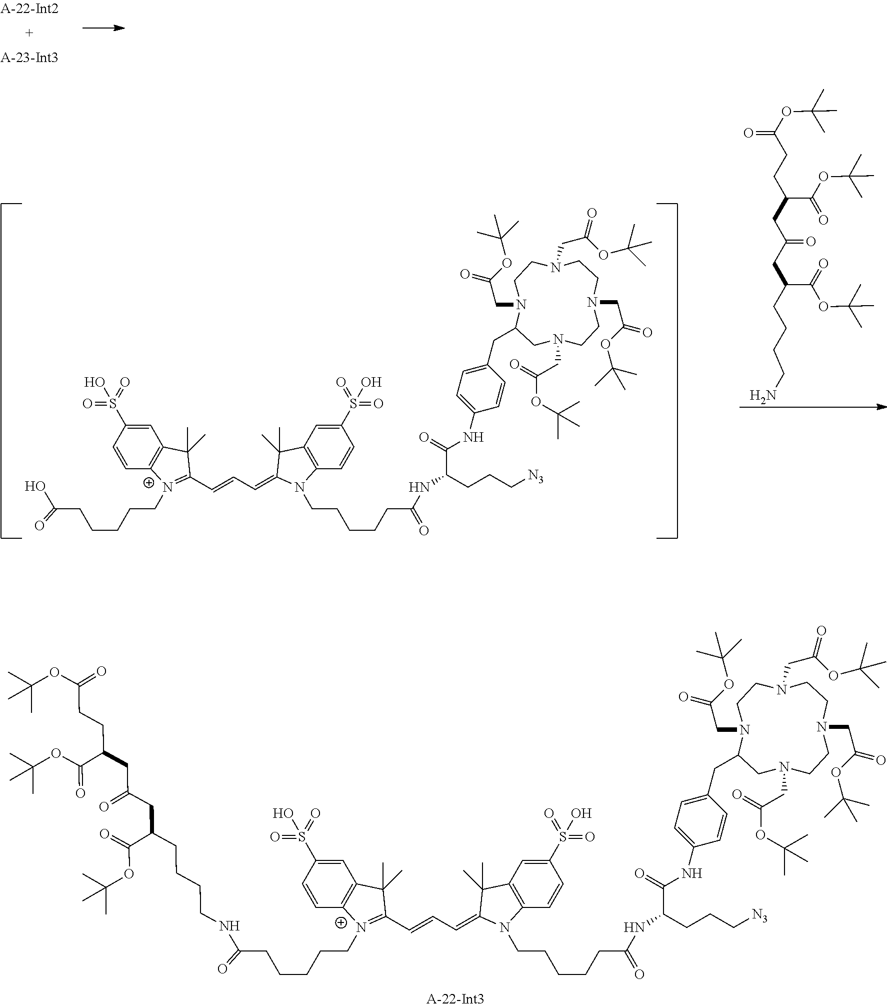

2-((E)-3-((E)-1-(6-(((2S)-5-azido-1-oxo-1-((4-((1,4,7,10-tetrakis(2-(tert-butoxy)-2-oxoethyl)-1,4,7,10-tetraazacyclododecan-2-yl)methyl)phenyl)amino)pentan-2-yl)amino)-6-oxohexyl)-3,3-dimethyl-5-sulfoindolin-2-ylidene)prop-1-en-1-yl)-1-(6-(((5S,9R)-12-(tert-butoxy)-5,9-bis(tert-butoxycarbonyl)-7,12-dioxododecyl)amino)-6-oxohexyl)-3,3-dimethyl-5-sulfo-3H-indol-1-ium salt (A-22-Int3) was synthesized from A-22-Int2 and A-23-Int3, followed by coupling with tri-tert-butyl (3R,7S)-11-amino-5-oxoundecane-1,3,7-tricarboxylate. To an empty 1.5 mL vial, A-22-Int2 (—1 mg, 311 mAu @210 nm) in 200 μL of N,N-dimethylformamide, A-23-Int3 (10 mg, 13.3 μmol) in 200 μL of N,N-dimethylformamide, 1-hydroxybenzotriazole (6 mg, 43 μmol), and 10 μL of pyridine were added before condensation was started with 1-Ethyl-3-(3-dimethylaminopropyl)carbodiimide (18 mg, 94 μmol). The condensation reaction was allowed to proceed at room temperature for 5 h. After 5 h, (S)-di-tert-butyl 2-(3-((S)-6-amino-1-(tert-butoxy)-1-oxohexan yl)ureido)pentanedioate 6 mg (12.3 μmol) and additional 1-Ethyl-3-(3-dimethylaminopropyl)carbodiimide 27 mg, 141 μmol) was added to the reaction mixture. The reaction was left at room temperature for 48h before the reaction was diluted with N,N-dimethylformamide (300 μL) and the mixture was loaded onto a preparative HPLC. HPLC separation took place on a 10 um C18(2) 250×21.2 mm 100A preparative column using a H2O/ACN (0.05% TFA), 20 min, 10% to 90% elution gradient at a flow rate of 15 mL/min. HPLC-MS: 1020.1 (M2+), retention time=18.0 min (250 nm).

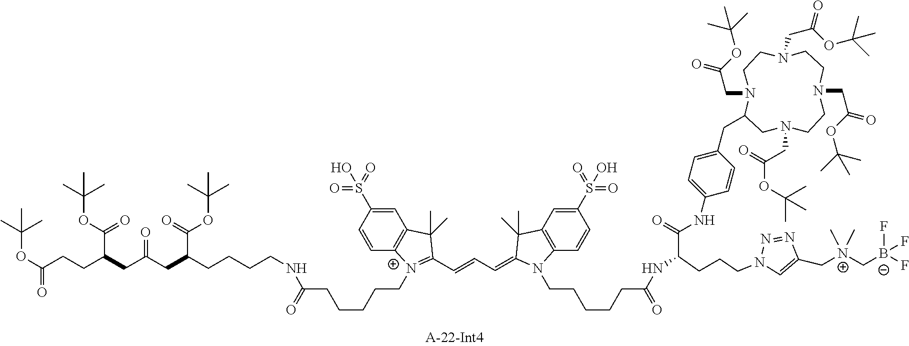

((((1-((4S)-4-(6-((E)-2-((E)-3-(1-(6-(((5S,9R)-12-(tert-butoxy)-5,9-bis(tert-butoxycarbonyl)-7,12-dioxododecyl)amino)-6-oxohexyl)-3,3-dimethyl-5-sulfo-3H-indol-1-ium-2-yl)allylidene)-3,3-dimethyl-5-sulfoindolin-1-yl)hexanamido)-5-oxo-5-((4-((1,4,7,10-tetrakis(2-(tert-butoxy)-2-oxoethyl)-1,4,7,10-tetraazacyclododecan-2-yl)methyl)phenyl)amino)pentyl)-1H-1,2,3-triazol-4-yl)methyl)dimethylammonio)methyl)-trifluoroborate (A-22-Int4) was synthesized from A-22-Int3. To an empty 1.5 mL vial, A-22-Int3 (˜1 mg, 311 mAu @210 nm) in 200 μL of N,N-dimethylformamide, N-Propargyl-N,N-dimethylammoniomethyl trifluoroborate (3 mg, 18 mg) and ascorbic acid (3.9 mg, 22 μmol) was added. A copper sulfate solution was prepared by dissolving copper (II) sulfate pentahydrate (1 mg, 4 μmol) in water (20 μL) in a second, separate 1.5 mL vial. The entirety of the copper sulfate solution was transferred into the first vial to initiate reaction. The reaction was left at room temperature for 16 h. After 16 hours, the reaction was diluted with N,N-dimethylformamide (1.3 mL) and the mixture was loaded onto a preparative HPLC. HPLC separation took place on a 10 um C18(2) 250×21.2 mm 100A preparative column using a H₂O/ACN (0.05% TFA), 20 min, 10% to 90% elution gradient at a flow rate of 15 mL/min. HPLC-MS: 1102.5 (M2+), retention time=16.0 min (550 nm).

Compound A-22-Int4 can serve as a precursor for Compound A-22.

### Example 4: Syntheses of Metal-Chelated Compounds A-22 and A-23

To use Compounds A-22-Int4 and A-23-Int4, they must first be placed in a state where they can capture metal ions, i.e., they must be transformed into Compounds A-22 and A-23, respectively, by removing t-butyl ester protecting groups.

For example, Compound A-22-Int4 is treated with neat trifluoroacetic acid (TFA) for 5 min. The resulting Compound A-22 is isolated by freezing (−20° C.), and then lyophilization to give the desired compound as a dry powder.

Two methods can be used to load metal ions into Compound A-22 or A-23: (1) An aqueous metal halide containing solution can be added directly to the trifluoroacetic acid solution described above used to generate Compound A-22 or A-23. (2) Metals load upon resuspension of the lyophilized product in water or buffered saline. Lyophilized powder containing Compound A-22 or A-23 can be resuspended in metal halide containing water or phosphate buffered saline. Metal chelation is complete within 5 min of incubation. To remove excess metal, final metal containing solutions are filtered through metal chelating column or cation exchange columns to remove free metal.

Compounds A-22 and A-23 are loaded with metals which can be gadolinium, gallium, lutecium, or actinium. All metal-chelated compounds can be used for fluorescent imaging, but only metal-chelated Compound A-22 can be used for PET imaging. Stable non-radioactive metals (Gd, Lu-175) are preferred for PET and fluorescent imaging, while Lu-177 and Ac-225 are preferred for radiation isotope therapy.

### Example 5: Medical Uses of Metal-Chelated Compounds A-22 and A-23

### Fluorescent Image Guided Surgery (FIGS) or Fluorescent Histopathology

Metal-chelated Compounds A-22 and A-23 can be used in fluorescent image guided surgery (FIGS) and fluorescent histopathology. Surgeons prefer to work with metals (M) that are inert and non-radioactive e.g. 175-Lu. Metal-chelated Compounds A-22 and A-23 are diluted with 1 mM phosphate buffered saline (1×PBS) and filtered through a 0.22 μm filter prior to injection. The resulting pH 7.4 filtrate is injected intravenously.

### PET Imaging

The radiolabeling of metal-chelated Compound A-22 is performed in one step under aqueous, acidic pH conditions (pH=2.0, pyridazine-HCl buffer, 10 μL) and proceeds quickly (10-15 min) at high temperatures (80-90° C.). Removal of unreacted [18F]-fluoride ion is performed by passing the metal-chelated Compound A-22 radiolabeling mixture through a prewashed (5 mL, deionized water) C18 cartridge (Waters no. 186005125). A 20-23 mL volume of water is used to flush contaminating [18F]-fluoride ion from the cartridge. [18F]-metal-chelated Compound A-22, bound to the cartridge, is eluted with a 4.0 mM HCl in ethanol (99%). Resulting [18F]-metal-chelated Compound A-22 in acidic ethanol is immediately diluted 10-fold with 1 mM phosphate buffered saline (1×PBS) and filtered through a 0.22 μm filter. The resulting pH 7.4 filtrate is injected intravenously through the cephalic vein in 3 to 5 mL solution. An average time of 73±27 minutes is allowed to pass between injection and imaging acquisition.

### Gd Contrast MRI Imaging

Gd-labeled Compound A-22 or A-23 can be used at high mass in MRI imaging. The Gd-labeled Compound A-22 or A-23 is diluted with 1 mM phosphate buffered saline (lx PBS) and filtered through a 0.22 μm filter. The resulting pH 7.4 filtrate is injected intravenously through the cephalic vein.

### Radiotherapy

Metal-labeled Compound A-22 or A-23, where the metal is ¹⁷⁷Lu or ²²⁵Ac can be used in radiation isotope therapy (RIT). The metal-labeled compound is diluted with 1 mM phosphate buffered saline (1×PBS) and filtered through a 0.22 μm filter. The resulting pH 7.4 filtrate is injected intravenously through the cephalic vein.

Those skilled in the art will recognize or be able to ascertain using no more than routine experimentation, many equivalents to the specific embodiments of the invention as described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

1. A compound comprising:
(i) a chelating ligand moiety (CL);
(ii) an optical probe moiety (OP); and
(iii) a biological targeting moiety (BT),
wherein one or more of the moieties of (i) to (iii) are affixed, bound, or connected by one or more linkers selected from L¹, L², L³, L⁴, L⁵, L⁶ and L⁷;
L¹ is a bond, -L^{1A}, -L^{1A}L^{1B}-, -L^{1A}L^{1B}-L^{1C}-, -L^{1A}C(O)NR¹¹L^{1B}-, -L^{1A}NR¹¹L^{1B}-, -L^{1A}C(O)L^{1B}-, -L^{1A}C(O)OL^{1B}-, -L^{1A}OC(O)L^{1B}-, -L^{1A}(OL^{1B})ₙ₁-, -L^{1A}NR¹¹C(O)L^{1B}-, -L^{1A}NR¹¹C(O)OL^{1B}-, or -L^{1A}NR¹¹(OL^{1B})ₙ₁-;
L² is a bond, -L^{2A}, -L^{2A}L^{2B}, -L^{2A}L^{2B}-L^{2C}-, -L^{2A}C(O)NR¹²L^{2B}-, -L^{2A}NR¹²L^{2B}-, -L^{2A}C(O)L^{2B}-, -L^{2A}C(O)OL^{2B}-, -L^{2A}OC(O)L^{2B}-, -L^{2A}(OL^{2B})ₙ₂-, -L^{2A}NR¹²C(O)L^{2B}-, -L^{2A}NR¹²C(O)OL^{2B}-, or -L^{2A}NR¹²(OL^{2B})ₙ₂-;
L³ is a bond, -L^{3A}, -L^{3A}L^{3B}- -L^{3A}L^{3B}-L^{3C}-, -L^{3A}C(O)NR¹³L^{3B}, -L^{3A}NR¹³L^{3B}-, -L^{3A}C(O)L^{3B}-, -L^{3A}C(O)OL^{3B}-, -L^{3A}OC(O)L^{3B}-, -L^{3A}(OL^{3B})ₙ₃-, -L^{3A}NR¹³C(O)L^{3B}-, -L^{3A}NR¹³C(O)OL^{3B}-, or -L^{3A}NR¹³(OL^{3B})ₙ₃-;
L⁴ is a bond, -L^{4A}, -L^{4A}L^{4B}-, -L^{4A}L^{4B}-L^{4C}-, -L^{4A}C(O)NR¹⁴L^{4B}-, -L^{4A}NR¹⁴L^{4B}-, -L^{4A}C(O)L^{4B}-, -L^{4A}C(O)OL^{4B}-, -L^{4A}OC(O)L^{4B}-, -L^{4A}(OL^{4B})ₙ₄-, -L^{4A}NR¹⁴C(O)L^{4B}-, -L^{4A}NR¹⁴C(O)OL^{4B}-, or -L^{4A}NR¹⁴(OL^{4B})ₙ₄-;
L⁵ is 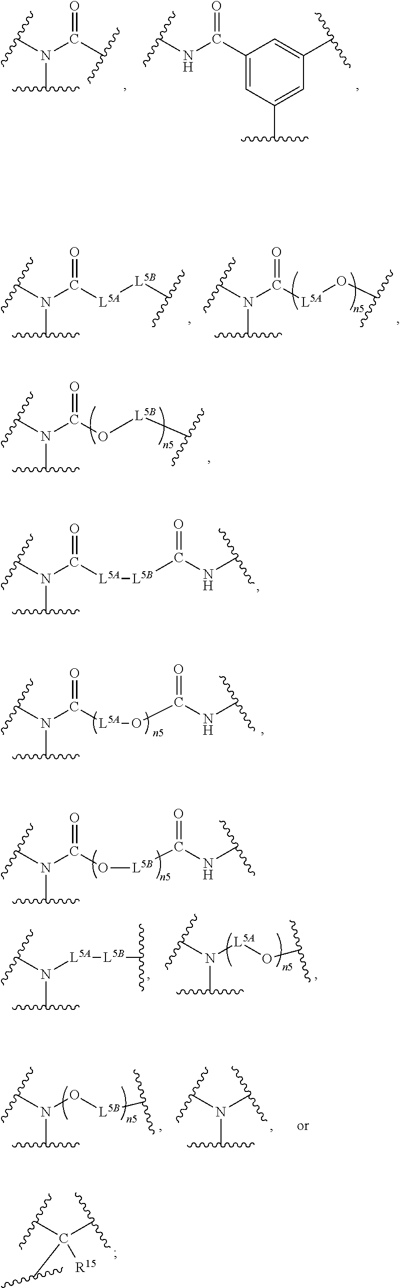
L⁶ is 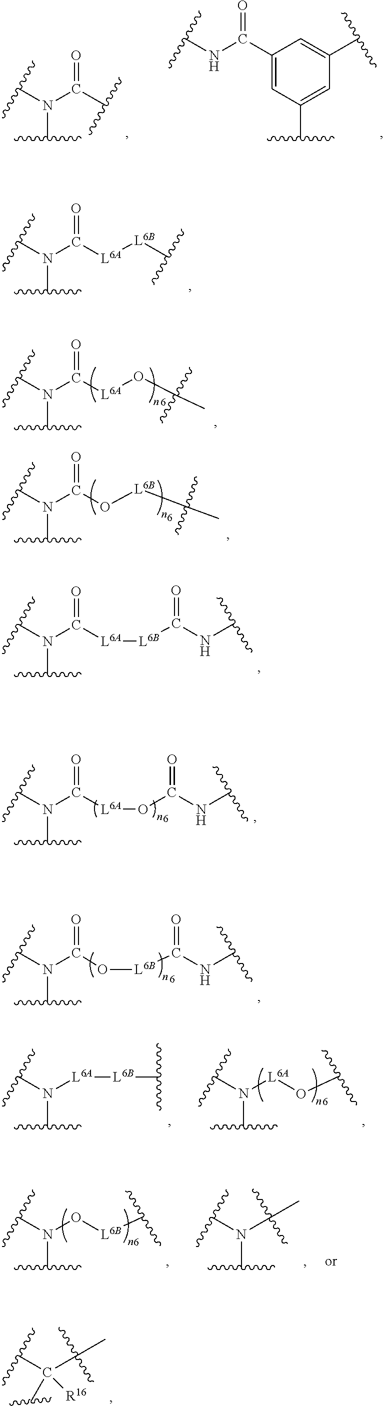
L⁷ is 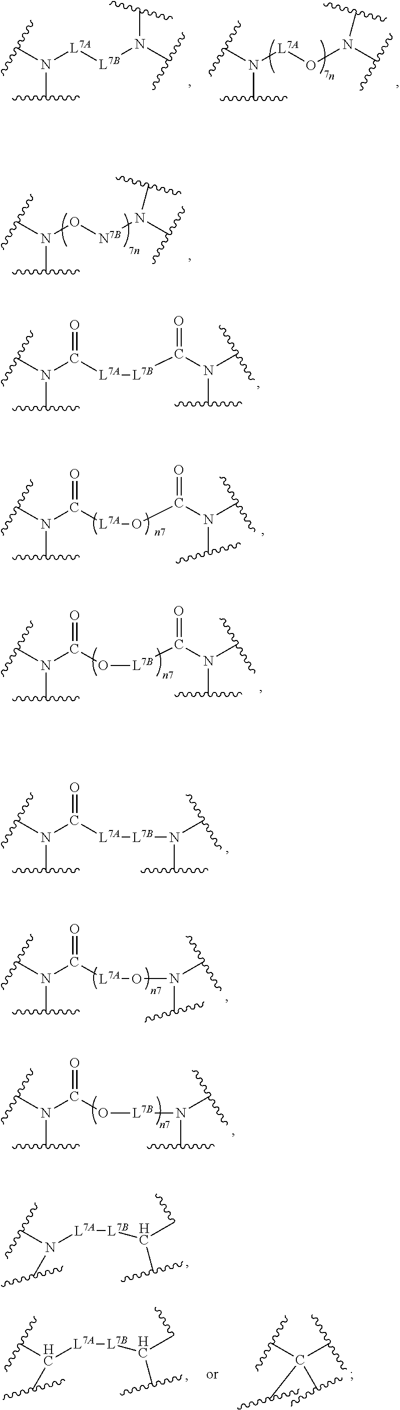
each L^{1A}, L^{1B}, L^{1C}, L^{2A}, L^{2B}, L^{2C}, L^{3A}, L^{3B}, L^{3C}, L^{4A}, L^{4B}, L^{4C}, L^{5A}, L^{5B}, L^{6A}, L^{6B}, L^{7A}, and L^{7B} is independently a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
each R¹¹, R¹², R¹³, R¹⁴, R¹⁵, and R¹⁶ is independently hydrogen, and unsubstituted alkyl; and
each n1, n2, n3, n4, n5, n6 and n7 is independently an integer from 0 to 20;
or a pharmaceutically acceptable salt thereof.

2. The compound of claim 1 comprising:
(i) a fluorine atom-carrying moiety (FCM);
(ii) a chelating ligand moiety (CL);
(iii) an optical probe moiety (OP); and
(iv) a biological targeting moiety (BT),
wherein one or more of the moieties of (i) to (iv) are affixed, bound, or connected by one or more linkers selected from L¹, L², L³, L⁴, L⁵, L⁶ and L⁷;
L¹ is a bond, -L^{1A}, -L^{1A}L^{1B}-, -L^{1A}L^{1B}-L^{1C}-, -L^{1A}C(O)NR¹¹L^{1B}-, -L^{1A}NR¹¹L^{1B}-, -L^{1A}C(O)L^{1B}-, -L^{1A}C(O)OL^{1B}-, -L^{1A}OC(O)L^{1B}-, -L^{1A}(OL^{1B})ₙ₁-, -L^{1A}NR¹¹C(O)L^{1B}-, -L^{1A}NR¹¹C(O)OL^{1B}-, or -L^{1A}NR¹¹(OL^{1B})ₙ₁-;
L² is a bond, -L^{2A}, -L^{2A}L^{2B}, -L^{2A}L^{2B}-L^{2C}-, -L^{2A}C(O)NR¹²L^{2B}-, -L^{2A}NR¹²L^{2B}-, -L^{2A}C(O)L^{2B}-, -L^{2A}C(O)OL^{2B}-, -L^{2A}OC(O)L^{2B}-, -L^{2A}(OL^{2B})ₙ₂-, -L^{2A}NR¹²C(O)L^{2B}-, -L^{2A}NR¹²C(O)OL^{2B}-, or -L^{2A}NR¹²(OL^{2B})ₙ₂-;
L³ is a bond, -L^{3A}, -L^{3A}L^{3B}- -L^{3A}L^{3B}-L^{3C}-, -L^{3A}C(O)NR¹³L^{3B}, -L^{3A}NR¹³L^{3B}-, -L^{3A}C(O)L^{3B}-, -L^{3A}C(O)OL^{3B}-, -L^{3A}OC(O)L^{3B}-, -L^{3A}(OL^{3B})ₙ₃-, -L^{3A}NR¹³C(O)L^{3B}-, -L^{3A}NR¹³C(O)OL^{3B}-, or -L^{3A}NR¹³(OL^{3B})ₙ₃-;
L⁴ is a bond, -L^{4A}, -L^{4A}L^{4B}-, -L^{4A}L^{4B}-L^{4C}-, -L^{4A}C(O)NR¹⁴L^{4B}-, -L^{4A}NR¹⁴L^{4B}-, -L^{4A}C(O)L^{4B}-, -L^{4A}C(O)OL^{4B}-, -L^{4A}OC(O)L^{4B}-, -L^{4A}(OL^{4B})ₙ₄-, -L^{4A}NR¹⁴C(O)L^{4B}-, -L^{4A}NR¹⁴C(O)OL^{4B}-, or -L^{4A}NR¹⁴(OL^{4B})ₙ₄-;
L⁵ is 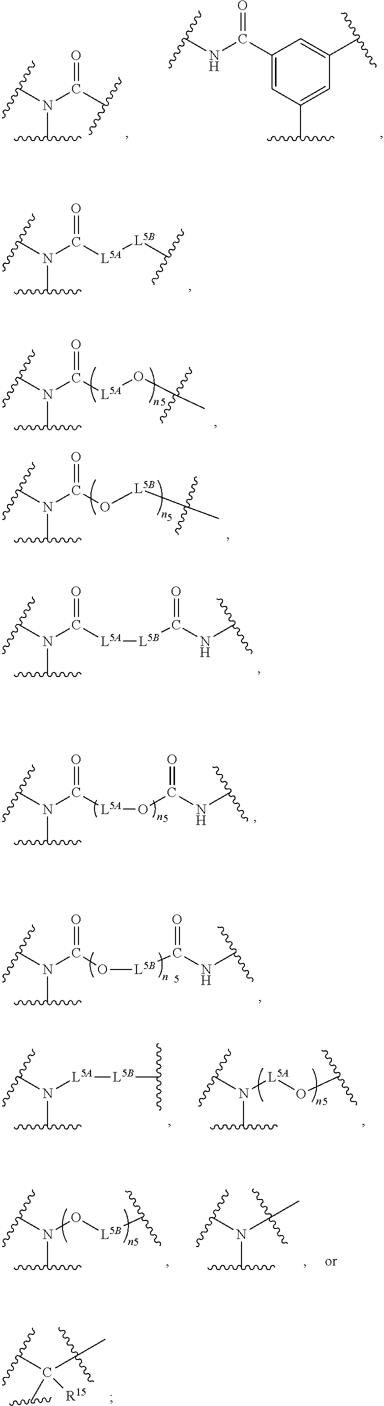
L⁶ is 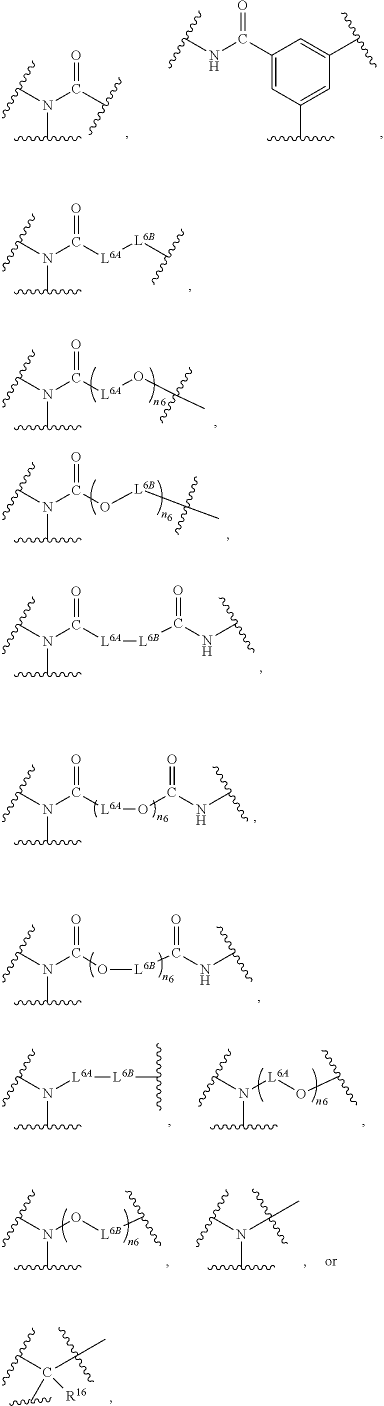
L⁷ is 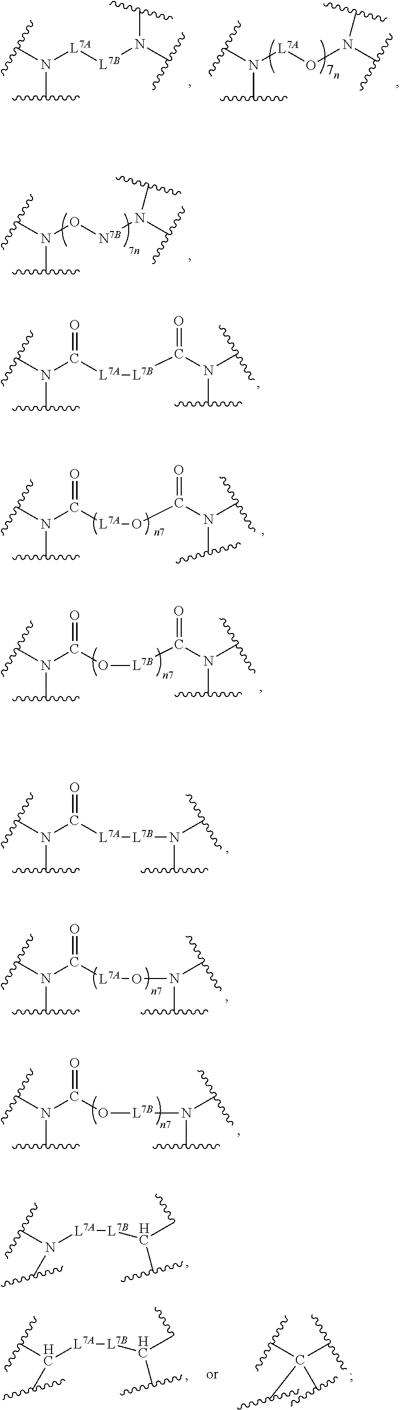
each L^{1A}, L^{1B}, L^{1C}, L^{2A}, L^{2B}, L^{2C}, L^{3A}, L^{3B}, L^{3C}, L^{4A}, L^{4B}, L^{4C}, L^{5A}, L^{5B}, L^{6A}, L^{6B}, L^{7A}, and L^{7B} is independently a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
each R¹¹, R¹², R¹³, R¹⁴, R¹⁵, and R¹⁶ is independently hydrogen, and unsubstituted alkyl; and
each n1, n2, n3, n4, n5, n6 and n7 is independently an integer from 0 to 20;
or a pharmaceutically acceptable salt thereof.

3. The compound of claim 2, or a pharmaceutically acceptable salt thereof, further comprising a metal ion bound to the chelating moiety (CL).

4.-7. (canceled)

8. The compound of claim 2, or a pharmaceutically acceptable salt thereof, wherein the FCM comprises

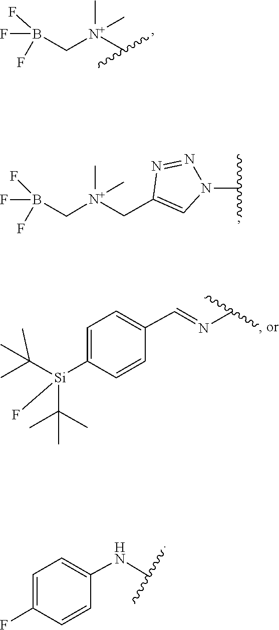

9.-10. (canceled)

11. The compound of claim 1, or a pharmaceutically acceptable salt thereof, wherein the OP comprises one or more fluorophores selected from trimethine cyanine (Cy3), pentamethine cyanine (Cy5), heptamethine cyanine (Cy7) cyanine, rhodamine, Evans blue (tetrasodium salt of 6,6′-{(3,3′-dimethyl[1,1′-biphenyl]-4,4′-diyl)bis[diazene-2,1-diyl]}bis(4-amino-5-hydroxynaphthalene-1,3-disulfonate), and isosulfan blue (lymphazurin).

12.-13. (canceled)

14. The compound of claim 1, or a pharmaceutically acceptable salt thereof, wherein the BT is

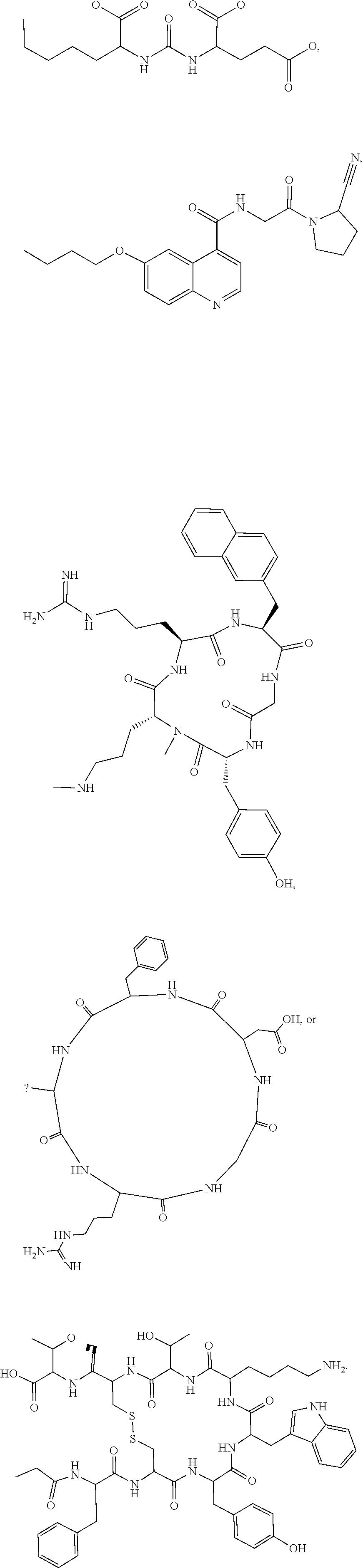

15. The compound of claim 1, or a pharmaceutically acceptable salt thereof, wherein the CL comprises one or more moieties selected from dodecane tetraacetic acid (DOTA), nitro-DOTA, 4-aminophenylethyl-1,4,7,10-tetraazacyclodecane-N,N′,N″,N′″-tetraacetic add (PA-DOTA), diethylenetriaminepentaacetic acid (DTPA), (2-[4,7-bis(carboxymethyl)-1,4,7-triazonan-1-yl]acetic acid) NOTA, (triethylenetetramine) TETA, desferrioxamine, (ethylenediaminetetraacetic acid) EDTA, and penicillamine.

16. The compound of claim 1, or a pharmaceutically acceptable salt thereof, wherein the metal ion is a cation of a metal selected from ¹⁷⁷Lu, ²²⁵Ac, Ga, Cu, Sm, Ra, Y, Pd, Ir, and Pb.

17. The compound of claim 1, or a pharmaceutically acceptable salt thereof, wherein the compound has a structure of:

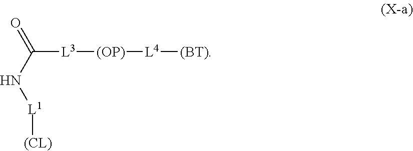

18. The compound of claim 1, or a pharmaceutically acceptable salt thereof, wherein the compound has a structure of:

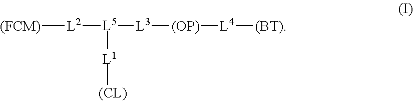

19.-25. (canceled)

26. The compound of claim 1 having the structure: 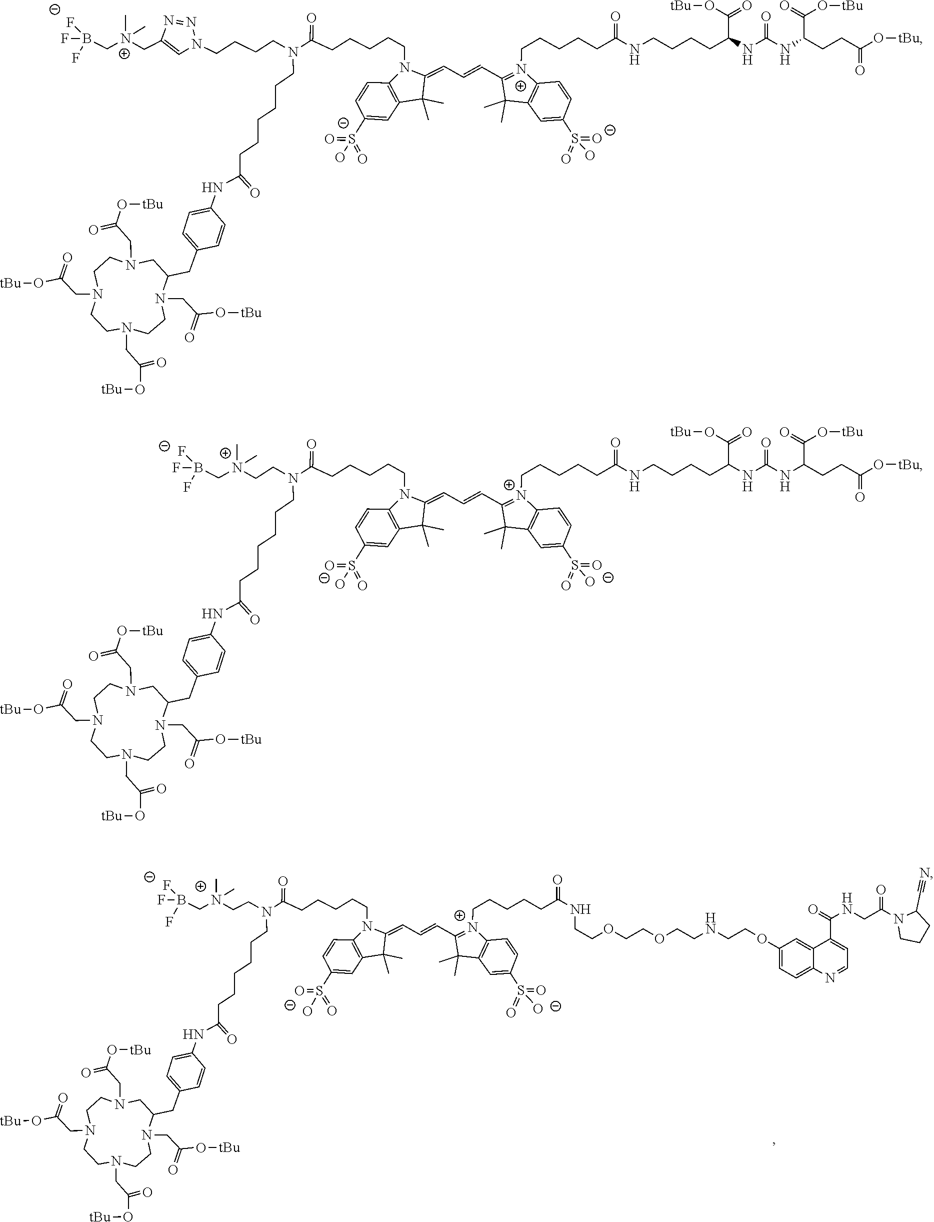 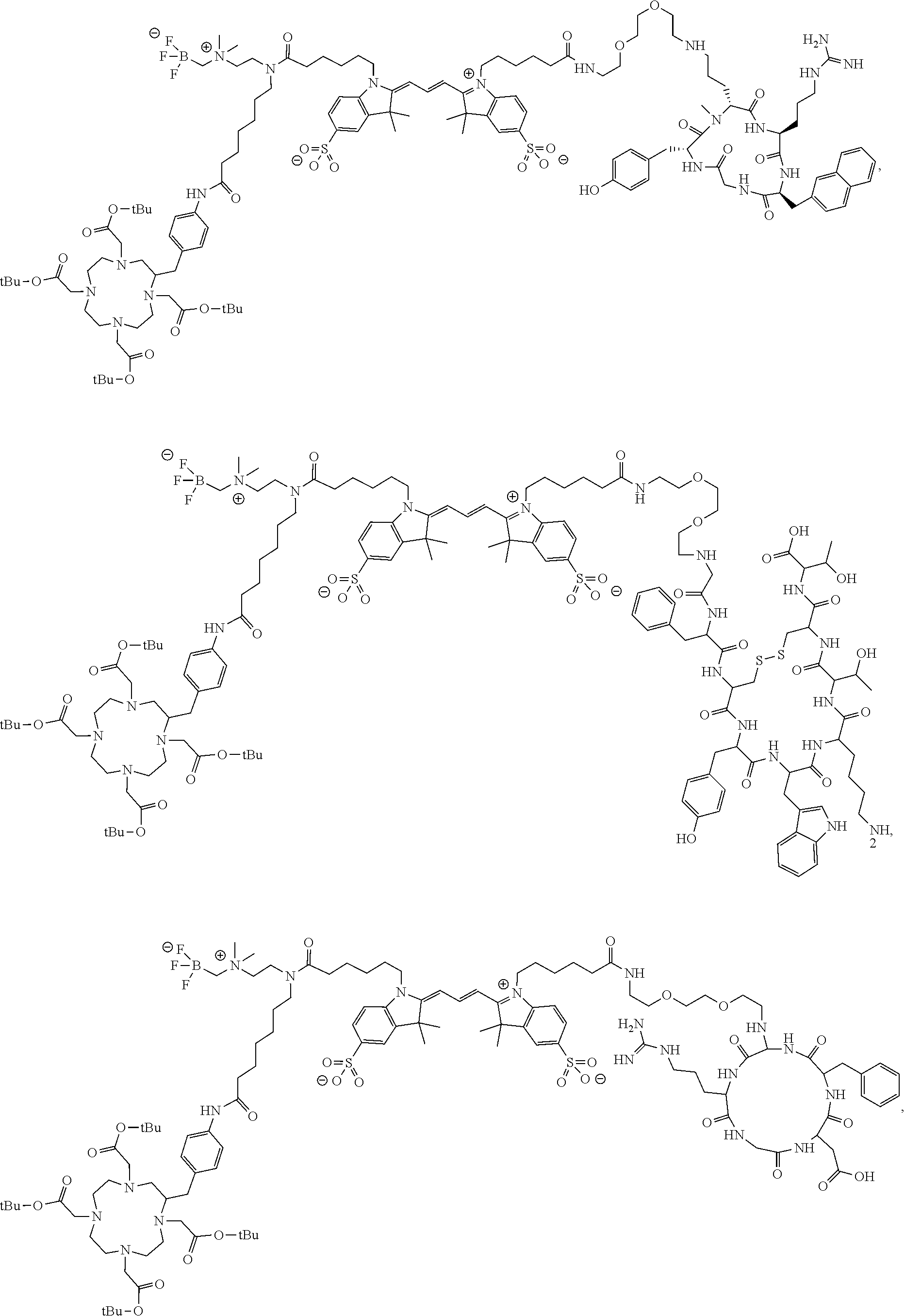 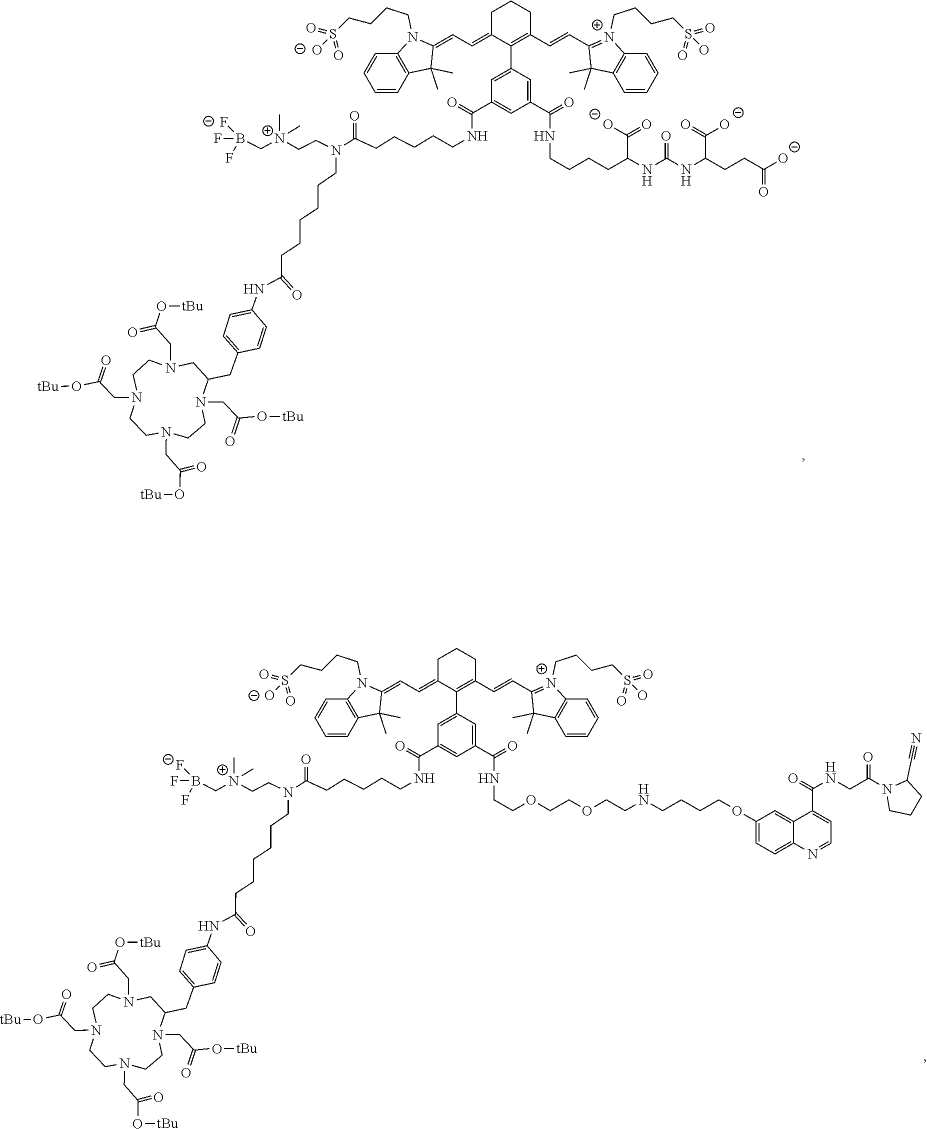 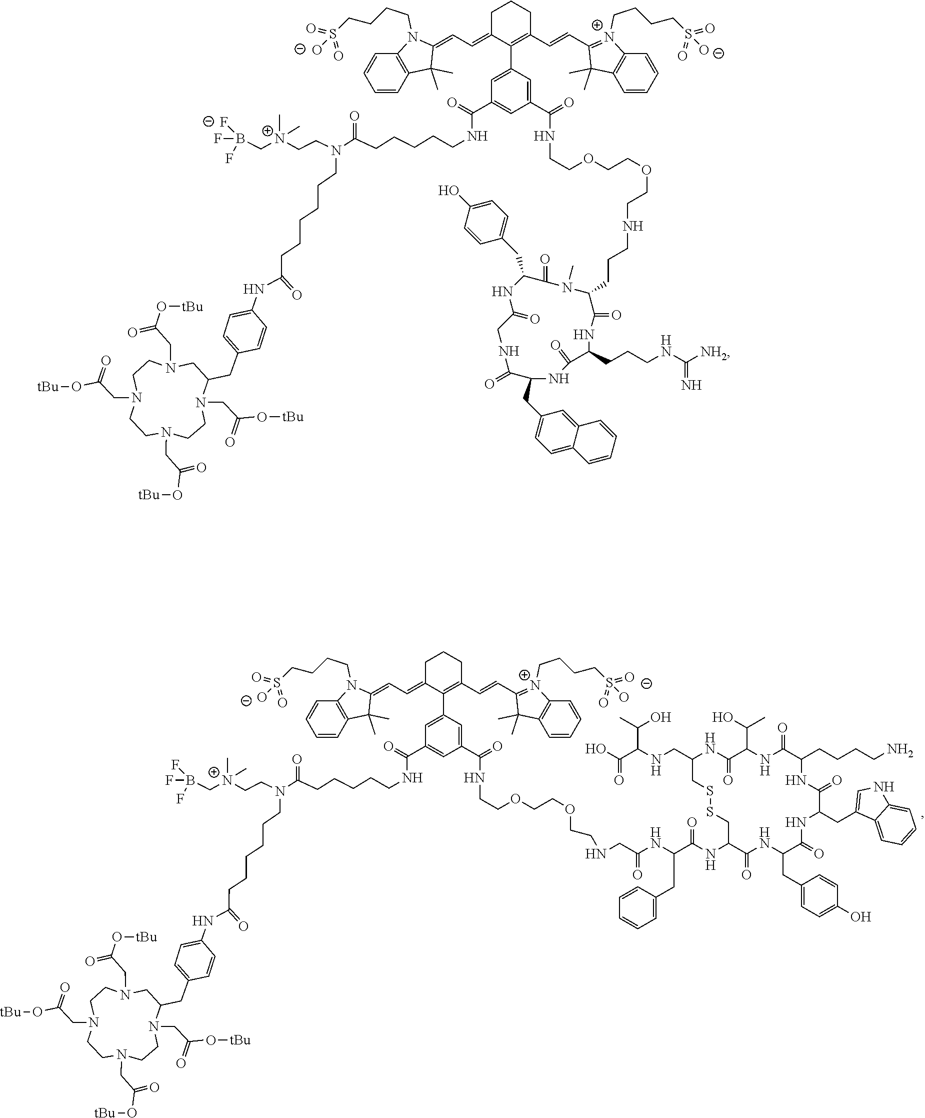 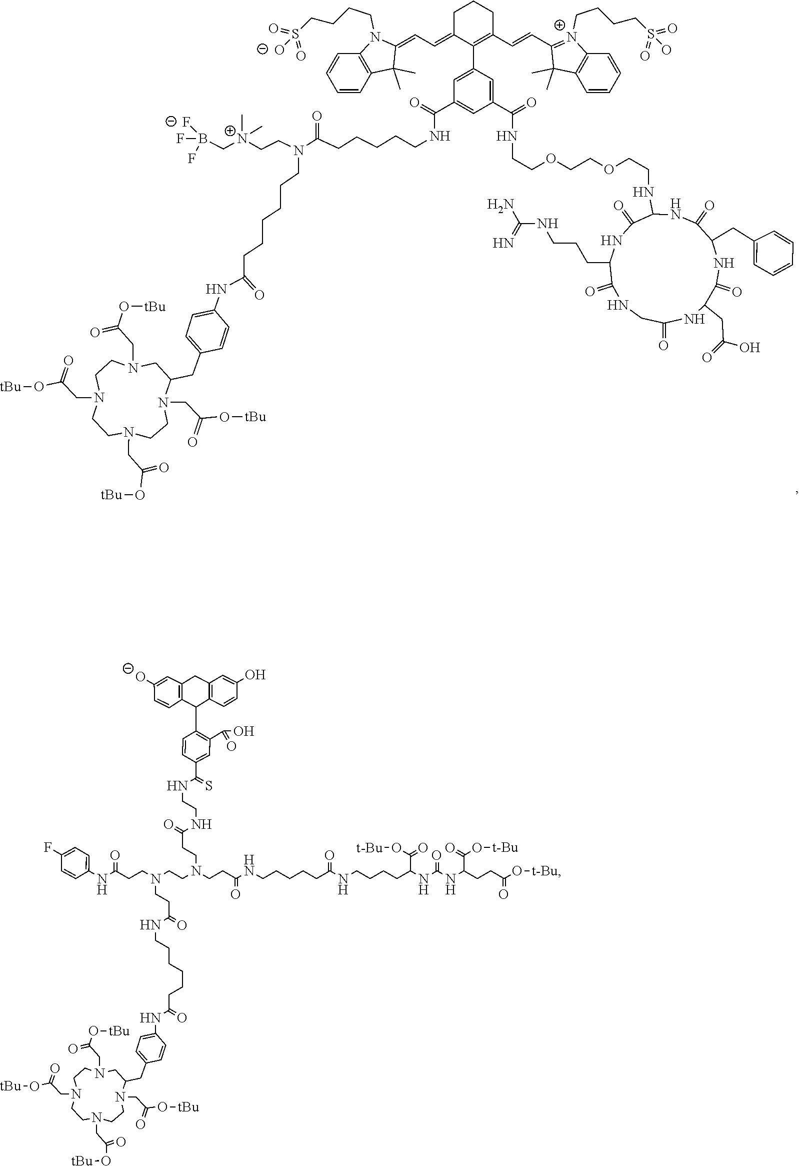 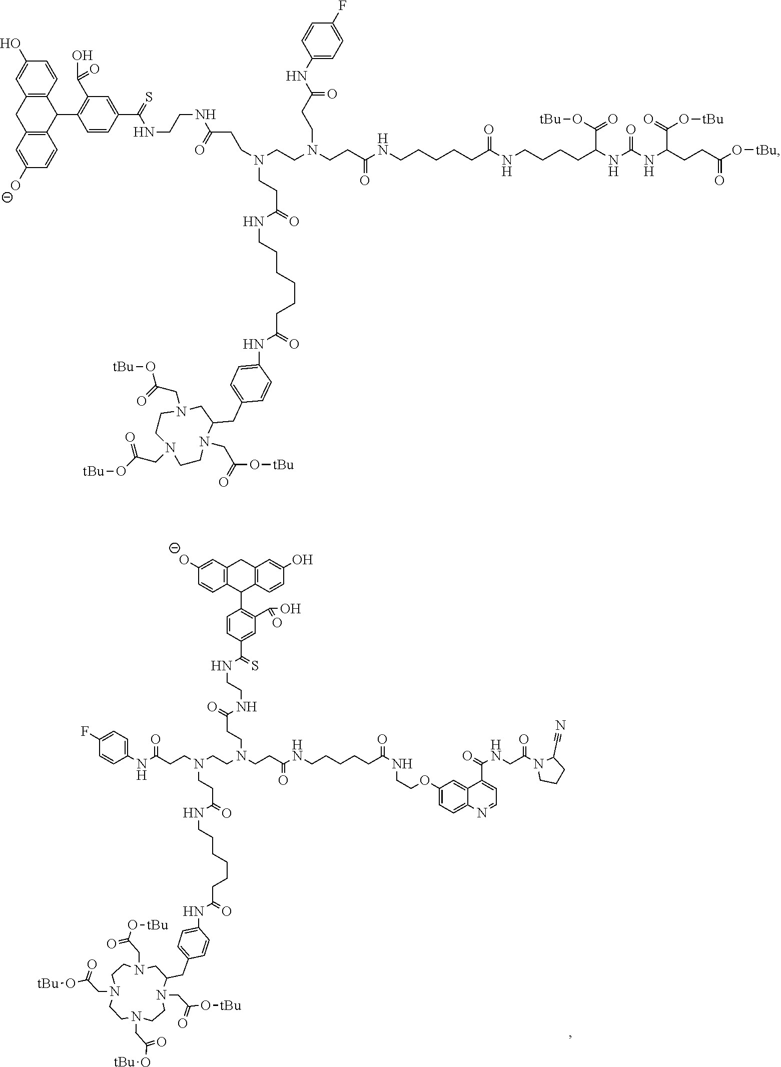 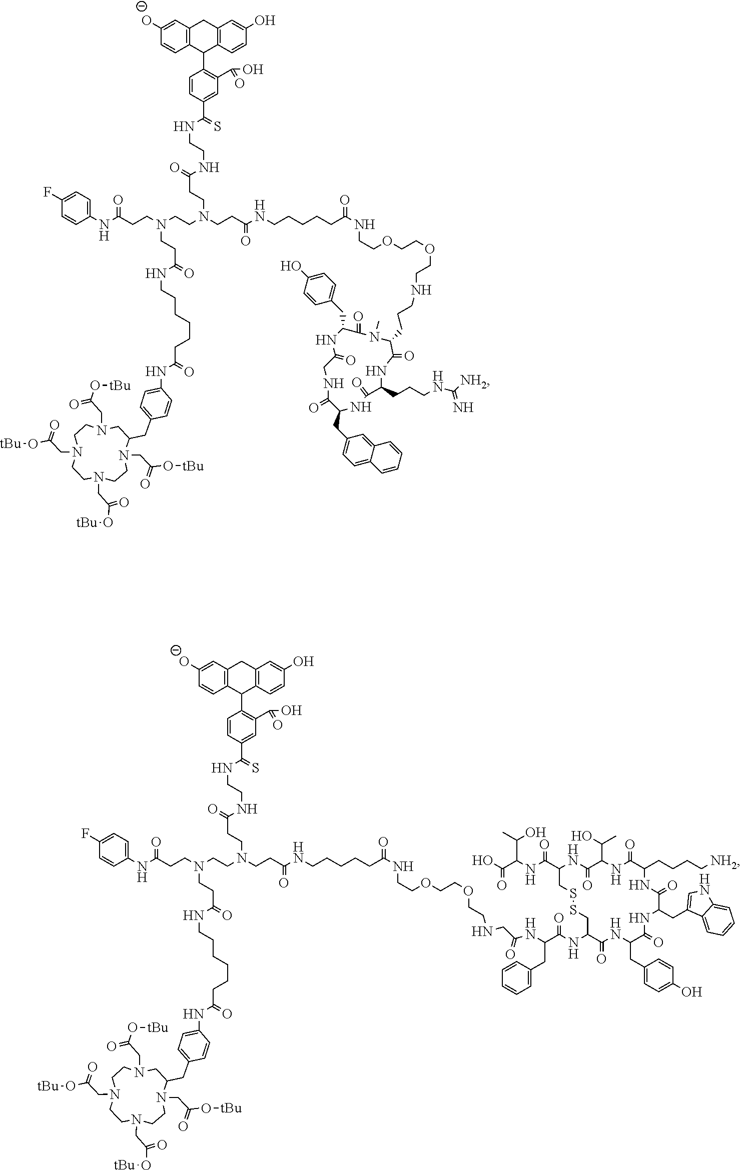 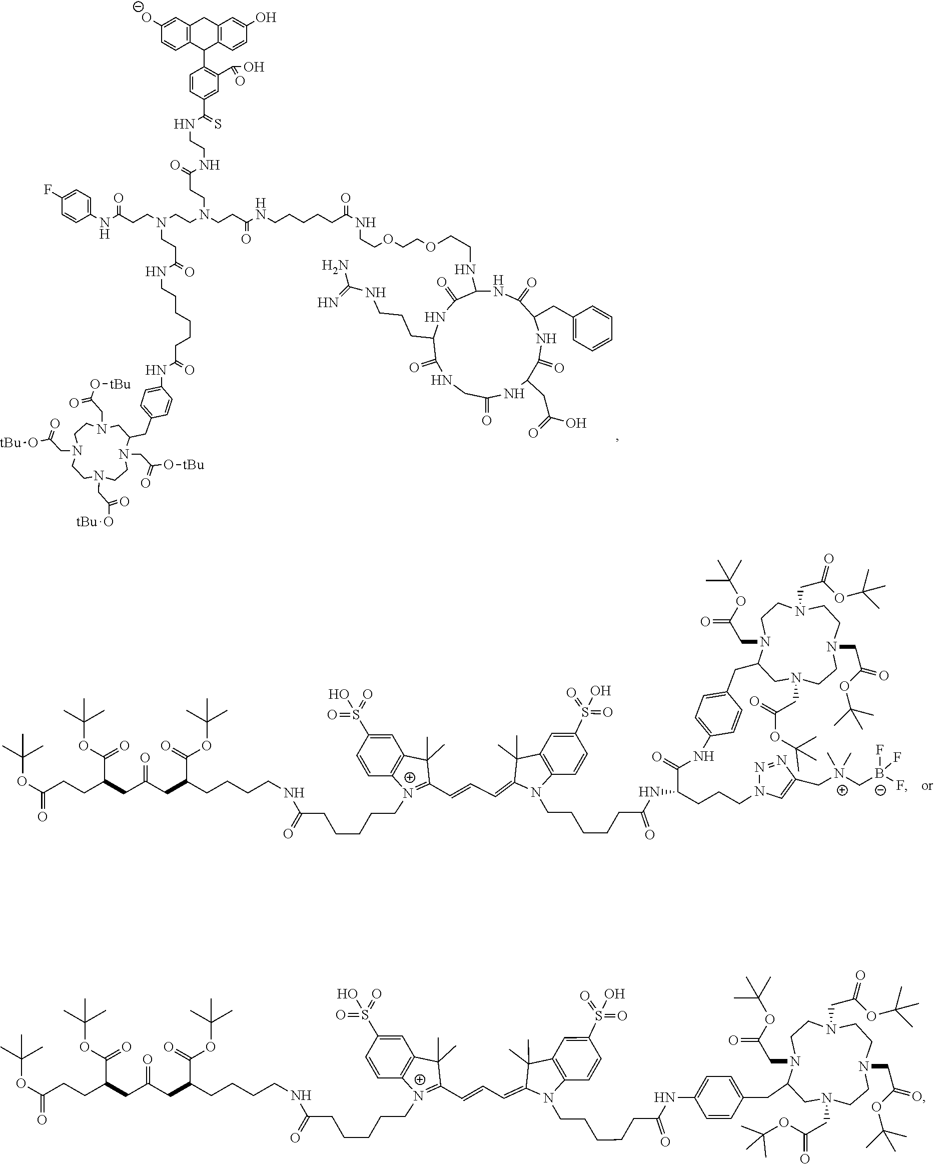
or a pharmaceutically acceptable salt thereof.

27. The compound of claim 1, having the structure: 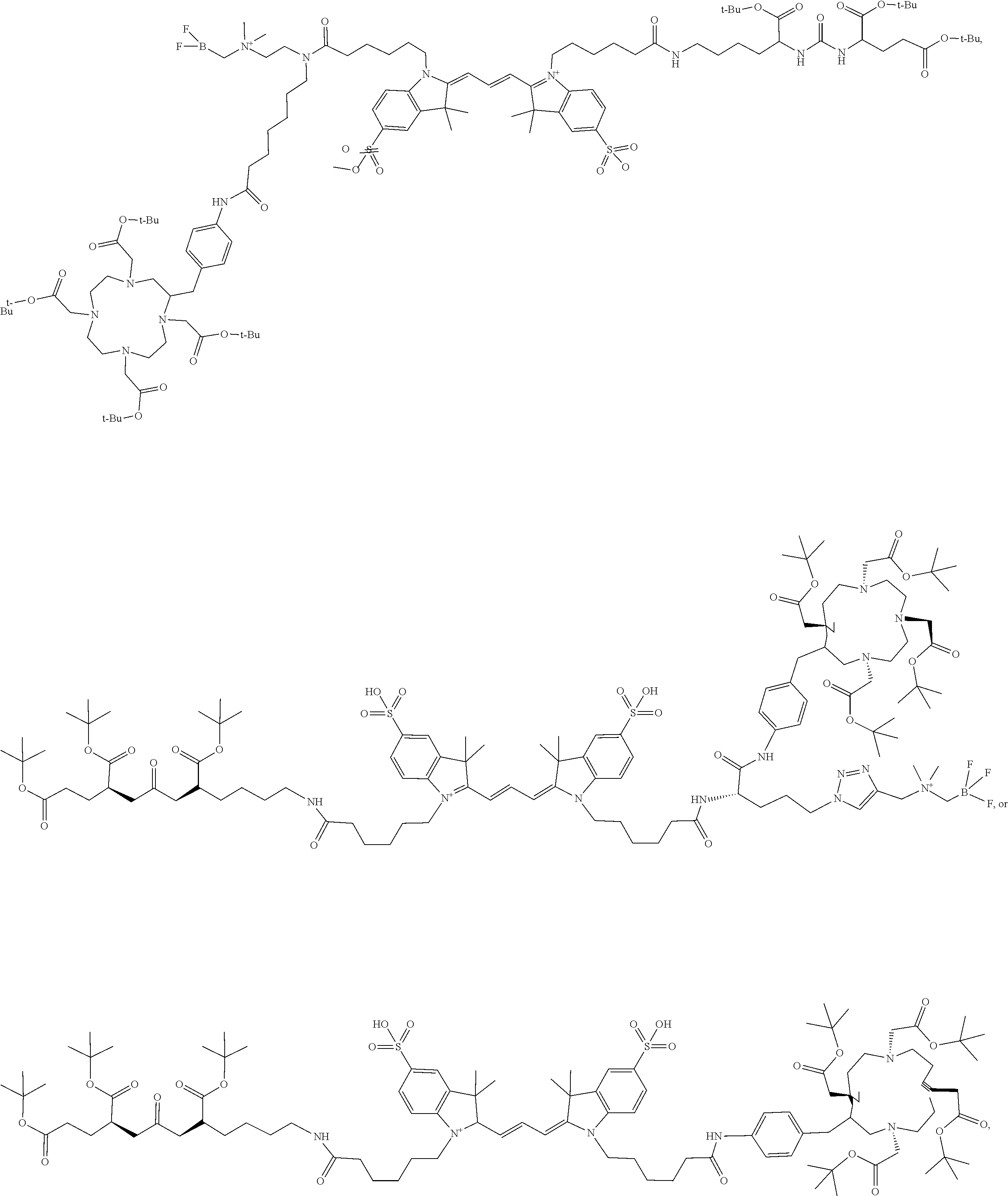
or a pharmaceutically acceptable salt thereof.

28. The compound of claim 18, wherein the compound is 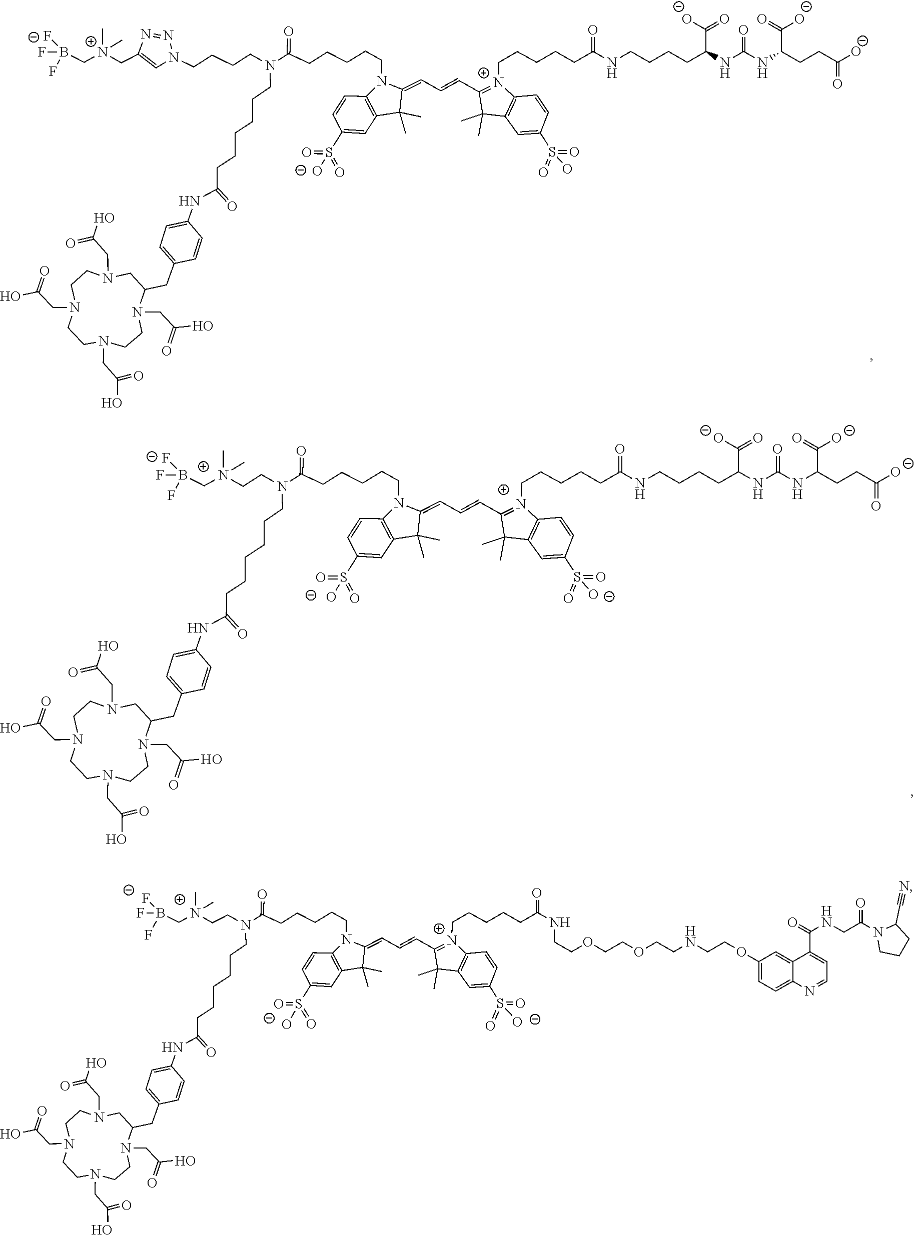 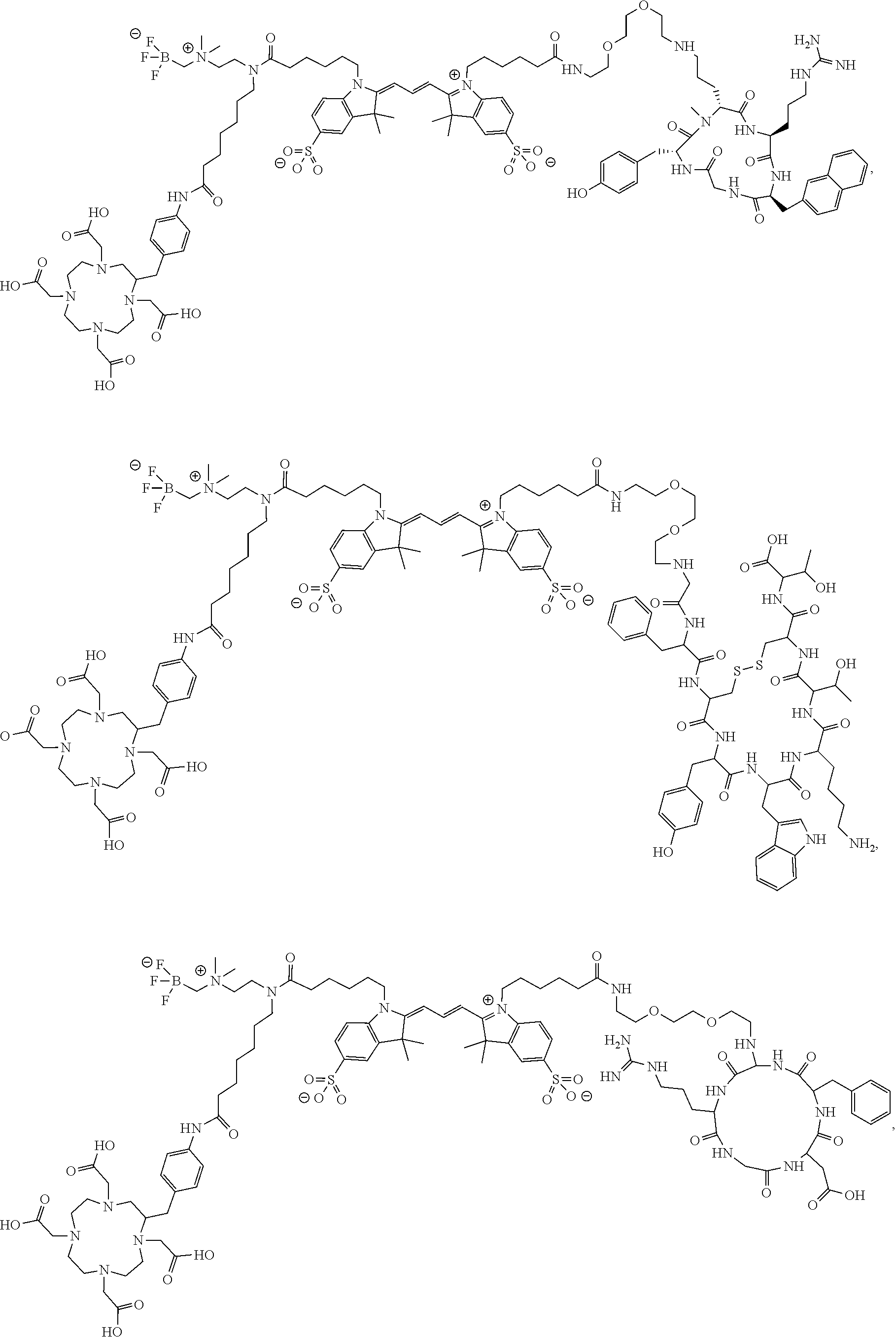 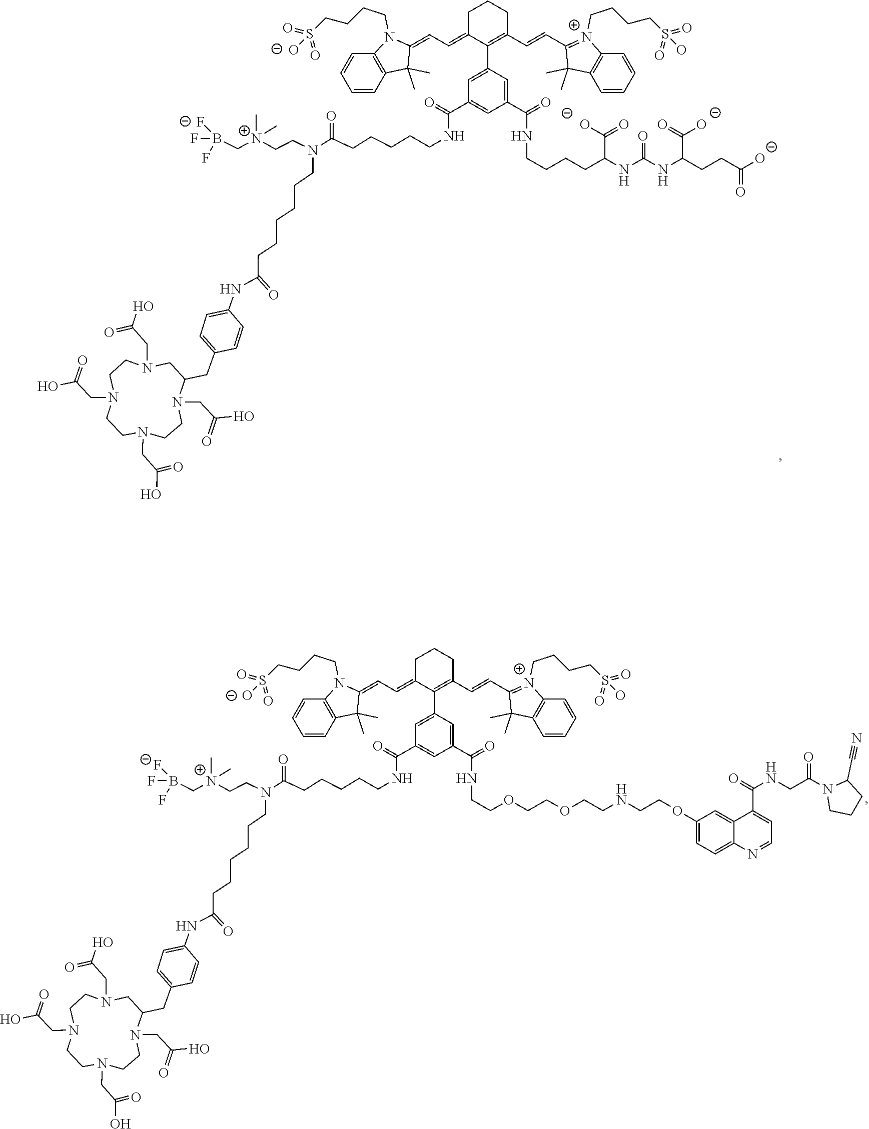 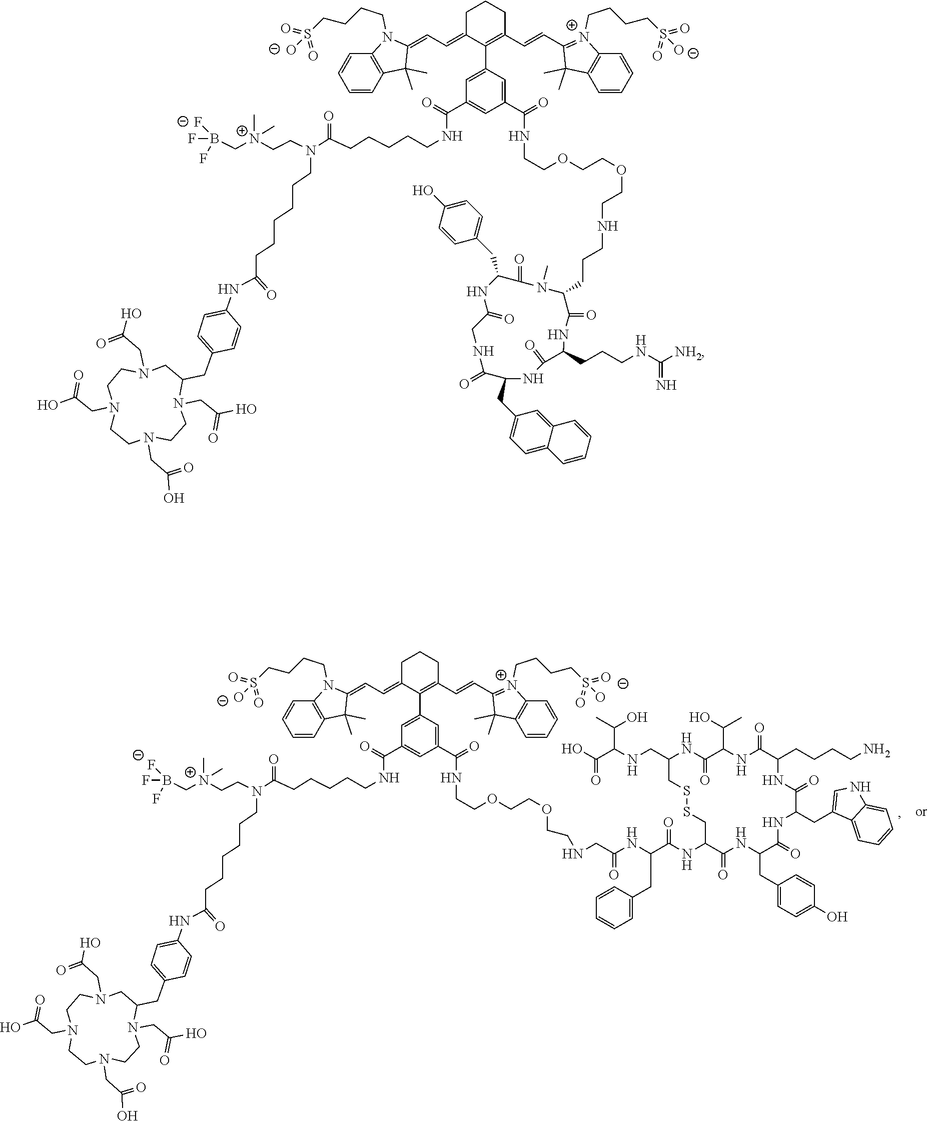 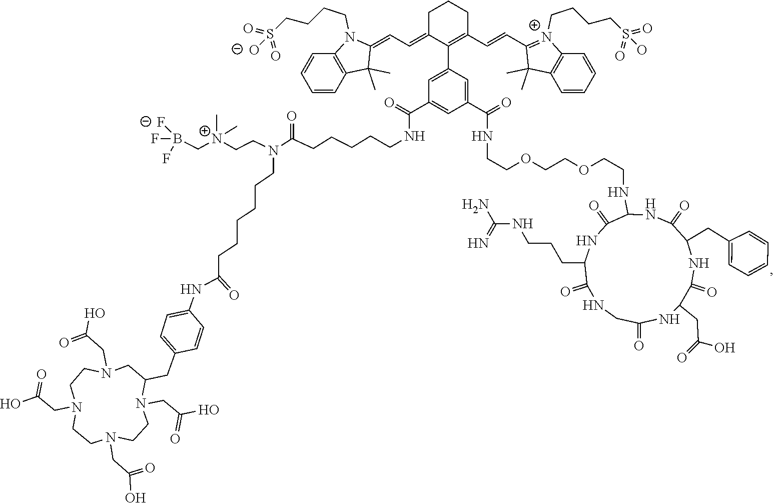
or a pharmaceutically acceptable salt thereof.

29. The compound of claim 17, wherein the compound is 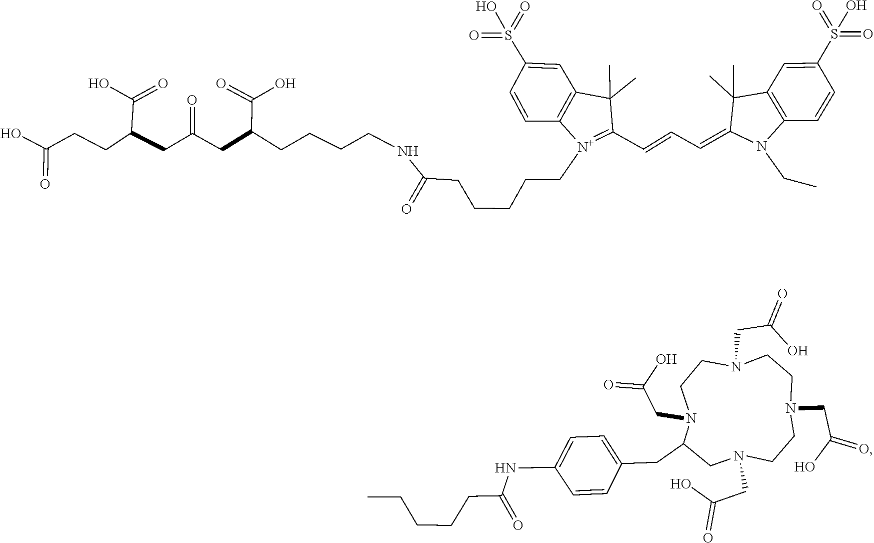
or a pharmaceutically acceptable salt thereof.

30.-38. (canceled)

31. The compound of claim 1, wherein the compound is 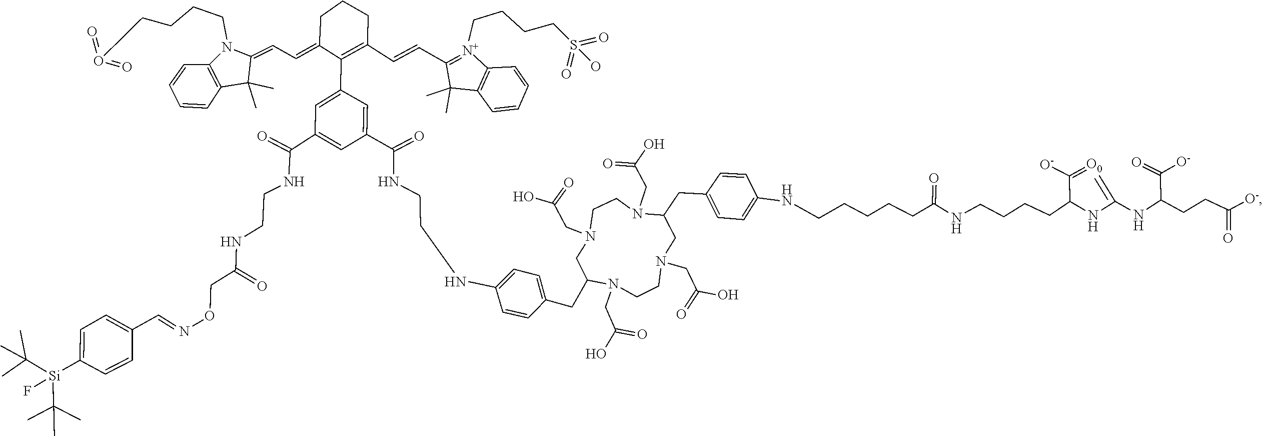
or a pharmaceutically acceptable salt thereof.

32.-46. (canceled)

47. The compound of claim 1, wherein the compound is 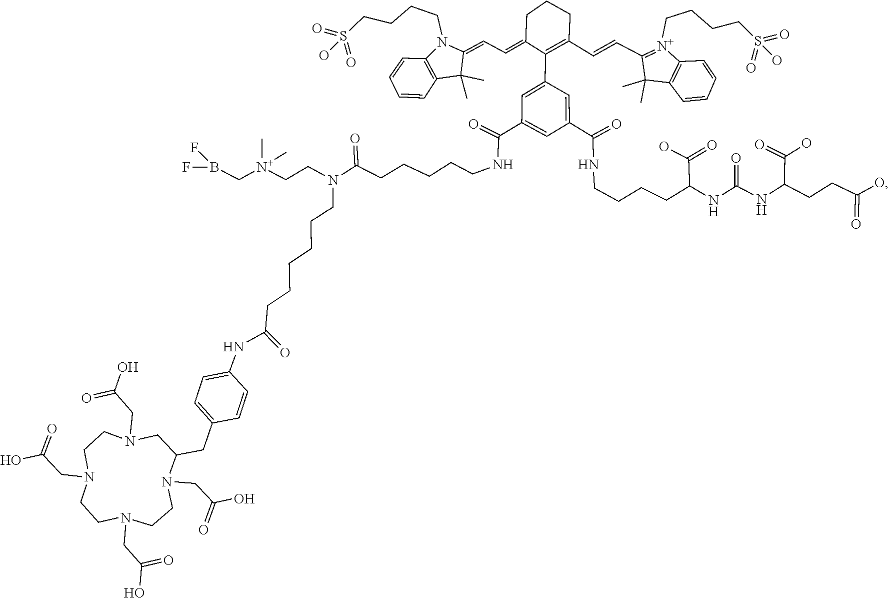
or a pharmaceutically acceptable salt thereof.

48.-54. (canceled)

55. The compound of claim 1, wherein the compound is 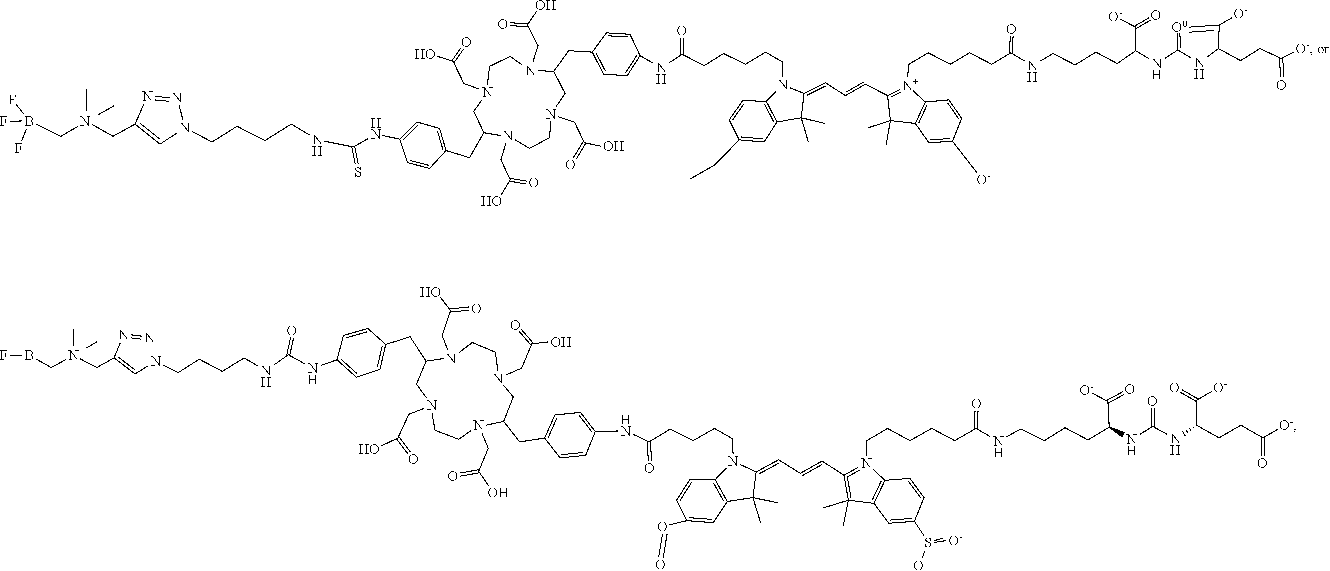
or a pharmaceutically acceptable salt thereof.

56. The compound of claim 1, wherein the compound is 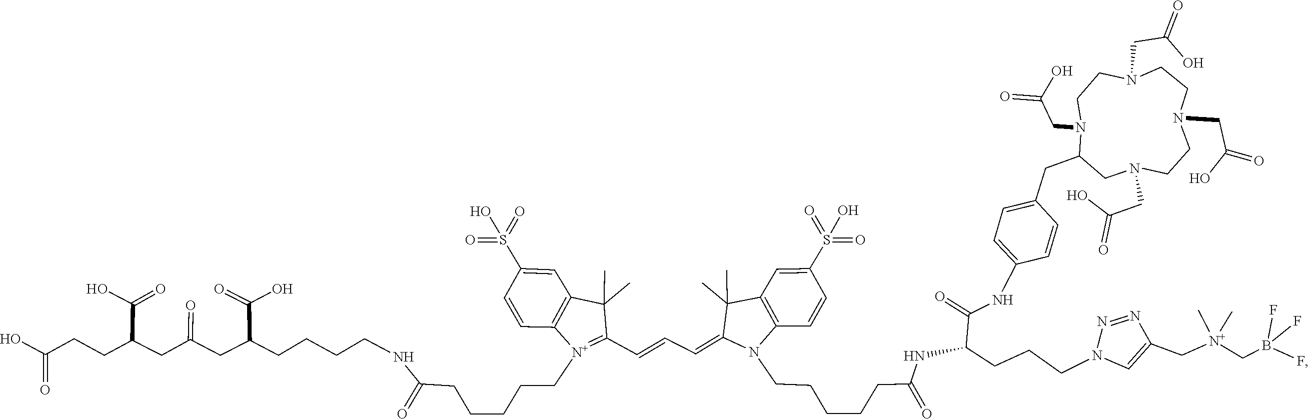
or a pharmaceutically acceptable salt thereof.

57.-66. (canceled)

67. The compound of claim 1, wherein the compound is 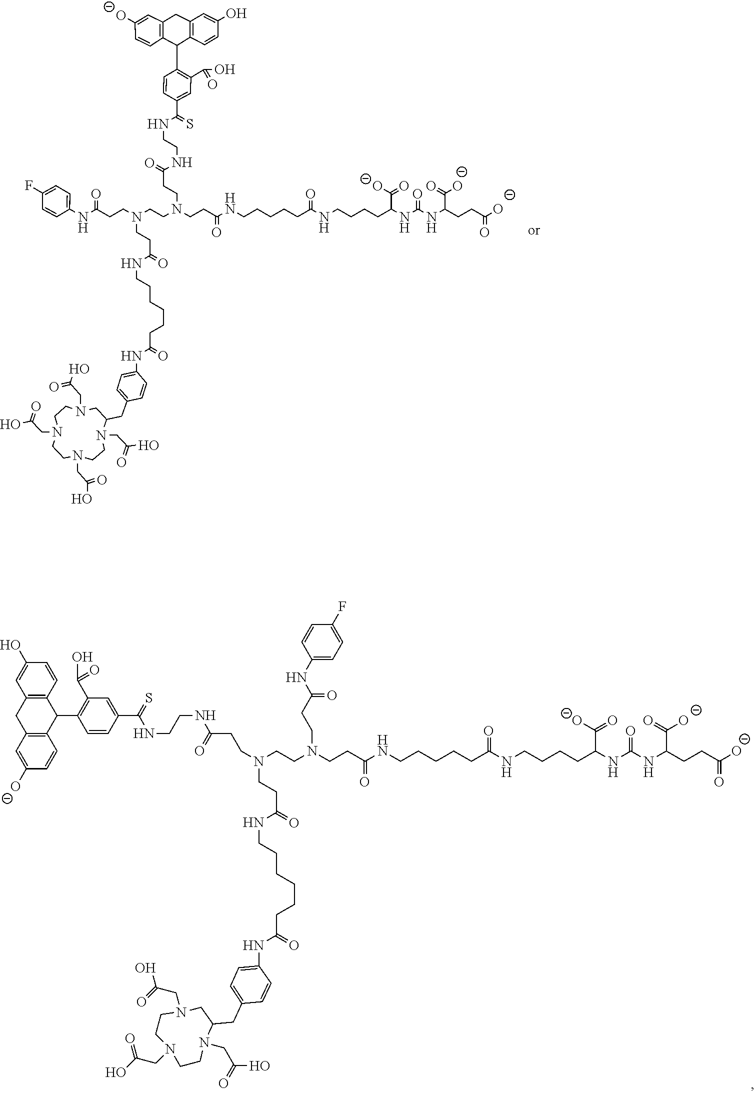 [SMILES: O=C(O)CCC(NC(=O)NC(CCCCNC(=O)CCCCCNC(=O)CCN(CCC(=O)NCCNC(=S)c1ccc(C2c3ccc([O-])cc3Cc3cc(O)ccc32)c(C(=O)O)c1)CCN(CCC(=O)NCCCCCCC(=O)Nc1ccc(CC2CN(CC(=O)O)CCN(CC(=O)O)CCN(CC(=O)O)CCN2CC(=O)O)cc1)CCC(=O)Nc1ccc(F)cc1)C(=O)[O-])C(=O)O ; O=C([O-])CCC(NC(=O)NC(CCCCNC(=O)CCCCCNC(=O)CCN(CCC(=O)Nc1ccc(F)cc1)CCN(CCC(=O)NCCCCCCC(=O)Nc1ccc(CC2CN(CC(=O)O)CCN(CC(=O)O)CCN2CC(=O)O)cc1)CCC(=O)NCCNC(=S)c1ccc(C2c3ccc([O-])cc3Cc3cc(O)ccc32)c(C(=O)O)c1)C(=O)[O-])C(=O)[O-]] 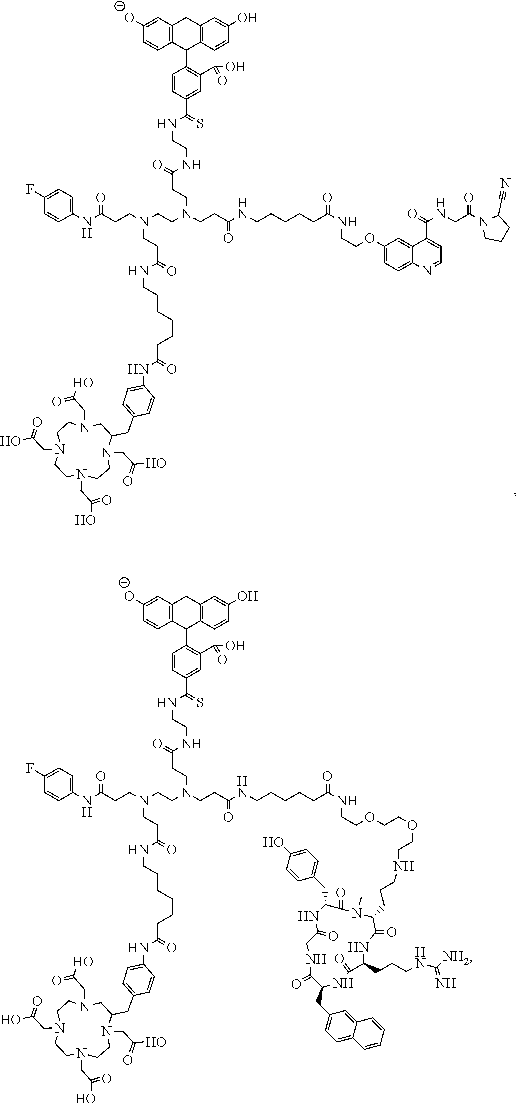 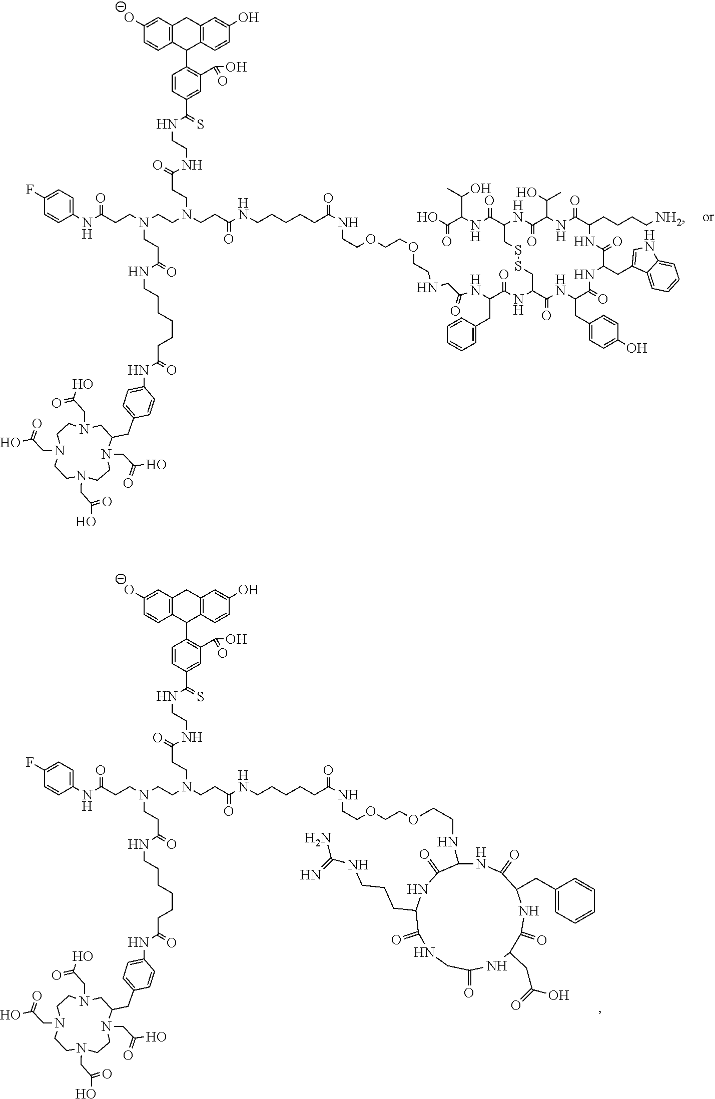
or a pharmaceutically acceptable salt thereof.

68.-69. (canceled)

70. A pharmaceutical composition comprising a compound of claim 1, or a pharmaceutically acceptable salt thereof, and a pharmaceutically acceptable excipient or carrier.

71.-76. (canceled)

77. A method for internal imaging of a biological tissue in a subject, comprising:
(i) administering a compound of claim 1 or a pharmaceutically acceptable salt thereof to the subject; and
(ii) imaging the biological tissue using a method comprising at least one of positron emission tomography (PET), single photon emission computer tomography (SPECT), magnetic resonance imaging (MRI), and gadolinium contrast magnetic resonance imaging (cMRI),
(iii) further comprising imaging the biological tissue with fluorescence-based optical imaging (FL).

78.-86. (canceled)

87. The method of claim 77, wherein the method is directed to imaging of prostate cancer tissue.

88. A method of treating cancer in a subject using radioisotope therapy, the method comprising administering a compound of claim 1 or a pharmaceutically acceptable salt thereof to the subject, wherein the compound comprises a radioisotope suitable for radioisotope therapy.

89. (canceled)
